SEQUENCE LISTING (23 sequences)

SEQ ID NO: 1 - DNA, 13 bp, Artificial Sequence
  Description of Artificial SequenceDNA sequence bound by peroxisome proliferator activated receptor (PPAR)-retinoid X receptor (RXR) heterodimer
aggtcanaggtca

SEQ ID NO: 2 - DNA, 18 bp, Artificial Sequence
  Description of Artificial Sequence Oligo(dT)-12-18 primer for reverse transcription of total RNA
tttttttttttttttttt

SEQ ID NO: 3 - DNA, 22 bp, Artificial Sequence
  Description of Artificial Sequenceperoxisome proliferator activated receptor alpha (PPARalpha) Real Time Quantitative PCR (RTQ PCR) primer F
cttcccaaagctccttcaaaaa

SEQ ID NO: 4 - DNA, 16 bp, Artificial Sequence
  Description of Artificial Sequenceperoxisome proliferator activated receptor alpha (PPARalpha) Real Time Quantitative PCR (RTQ PCR) primer R
ctgcgcatgctccgtg

SEQ ID NO: 5 - DNA, 21 bp, Artificial Sequence
  Description of Artificial Sequenceperoxisome proliferator activated receptor alpha (PPARalpha) fluorogenic probe P
tggtggaccttcggcagctgg

SEQ ID NO: 6 - DNA, 20 bp, Artificial Sequence
  Description of Artificial Sequenceperoxisome proliferator activated receptor delta (PPARdelta) Real Time Quantitative PCR (RTQ PCR) primer F
gatgacagtgacctggcgct

SEQ ID NO: 7 - DNA, 16 bp, Artificial Sequence
  Description of Artificial Sequenceperoxisome proliferator activated receptor delta (PPARdelta) Real Time Quantitative PCR (RTQ PCR) primer R
aggcctggccggtctc

SEQ ID NO: 8 - DNA, 24 bp, Artificial Sequence
  Description of Artificial Sequenceperoxisome proliferator activated receptor delta (PPARdelta) fluorogenic probe P
ttcatcgcggccatcattctgtgt

SEQ ID NO: 9 - DNA, 17 bp, Artificial Sequence
  Description of Artificial Sequenceperoxisome proliferator activated receptor gamma (PPARgamma) Real Time Quantitative PCR (RTQ PCR) primer F
agtggagaccgcccagg

SEQ ID NO: 10 - DNA, 21 bp, Artificial Sequence
  Description of Artificial Sequenceperoxisome proliferator activated receptor gamma (PPARgamma) Real Time Quantitative PCR (RTQ PCR) primer R
gcagcaggttgtcttggatgt

SEQ ID NO: 11 - DNA, 23 bp, Artificial Sequence
  Description of Artificial Sequenceperoxisome proliferator activated receptor gamma (PPARgamma) fluorogenic probe P
ttgctgaacgtgaagcccatcga

SEQ ID NO: 12 - DNA, 19 bp, Artificial Sequence
  Description of Artificial Sequencefatty acid translocase (FAT/CD36) Real Time Quantitative PCR (RTQ PCR) primer F
cggcgatgagaaagcagaa

SEQ ID NO: 13 - DNA, 17 bp, Artificial Sequence
  Description of Artificial Sequencefatty acid translocase (FAT/CD36) Real Time Quantitative PCR (RTQ PCR) primer R
caaccaggcccaggagc

SEQ ID NO: 14 - DNA, 30 bp, Artificial Sequence
  Description of Artificial Sequencefatty acid translocase (FAT/CD36) fluorogenic probe P
tgttcagaaaccaagtgaccgggaaaataa

SEQ ID NO: 15 - DNA, 20 bp, Artificial Sequence
  Description of Artificial Sequencefatty acid transport protein (FATP) Real Time Quantitative PCR (RTQ PCR) primer F
gcacagcaggtactaccgca

SEQ ID NO: 16 - DNA, 14 bp, Artificial Sequence
  Description of Artificial Sequencefatty acid transport protein (FATP) Real Time Quantitative PCR (RTQ PCR) primer R
ggcggcacgcatgc

SEQ ID NO: 17 - DNA, 24 bp, Artificial Sequence
  Description of Artificial Sequencefatty acid transport protein (FATP) fluorogenic probe P
tgctgcctttggccaccattccta

SEQ ID NO: 18 - DNA, 21 bp, Artificial Sequence
  Description of Artificial Sequenceintestinal fatty acid-binding protein (I-FABP) Real Time Quantitative PCR (RTQ PCR) primer F
tcaccatcacctatggaccca

SEQ ID NO: 19 - DNA, 19 bp, Artificial Sequence
  Description of Artificial Sequenceintestinal fatty acid-binding protein (I-FABP) Real Time Quantitative PCR (RTQ PCR) primer R
tccagttcgcactcctccc

SEQ ID NO: 20 - DNA, 25 bp, Artificial Sequence
  Description of Artificial Sequenceintestinal fatty acid-binding protein (I-FABP) fluorogenic probe P
agtggtccgcaatgagttcaccctg

SEQ ID NO: 21 - DNA, 18 bp, Artificial Sequence
  Description of Artificial Sequence glyceraldehyde 3-phosphate dehydrogenase (GAPDH) internal standard Real Time Quantitative PCR (RTQ PCR) primer F
tcactggcatggccttcc

SEQ ID NO: 22 - DNA, 17 bp, Artificial Sequence
  Description of Artificial Sequence glyceraldehyde 3-phosphate dehydrogenase (GAPDH) internal standard Real Time Quantitative PCR (RTQ PCR) primer R
ggcggcacgtcagatcc

SEQ ID NO: 23 - DNA, 24 bp, Artificial Sequence
  Description of Artificial Sequence glyceraldehyde 3-phosphate dehydrogenase (GAPDH) internal standard fluorogenic probe P
ttcctacccccaatgtgtccgtcg

(19) United States
(12) Patent Application Publication
(10) Pub. No.: US 2005/0054730 A1
(43) Pub. Date: Mar. 10, 2005

(54) Compounds, compositions and treatment of oleoylethanolamide-like modulators of PPARalpha

(75) Inventors: Jin Fu, Irvine, CA (US); Silvana Gaetani, Irvine, CA (US); Daniele Piomelli, Irvine, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/884,617

(22) Filed: Jul. 1, 2004

Related U.S. Application Data
(63) Continuation-in-part of application No. 10/112,509, filed on Mar. 27, 2002.
(60) Provisional application No. 60/485,062, filed on Jul. 2, 2003.
(60) Provisional application No. 60/336,289, filed on Oct. 31, 2001.
(60) Provisional application No. 60/279,542, filed on Mar. 27, 2001.

(51) Int. Cl. A61K 31/16

(52) U.S. Cl. 514/625

(57) ABSTRACT

The present invention provides compounds, compositions, and methods for the treatment of disorders and conditions mediated by PPARα. The invention relates to the surprising discovery that oleoylethanolamide (OEA) is an endogenous high affinity and selective ligand of PPARα. The compounds of the invention include, but are not limited to, specific PPARα agonists sharing the receptor binding properties of OEA and fatty acid alkanolamides and their homologs which also are PPARα agonists. Such OEA-like compounds include, but are not limited to, compounds of the following formula: 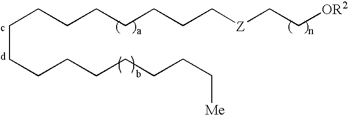
in which n is from 0 to 5, the sum of a and b can be from 0 to 4; Z is a member selected from the group consisting of —C(O)N(R^{o})—; —(R^{o})NC(O)—; —OC(O)—; —(O)CO—; O; NR^{o}; and S; and wherein R^{o} and R² are members independently selected from the group consisting of unsubstituted or unsubstituted alkyl, hydrogen, C₁-C₆ alkyl, and lower (C₁-C₆) acyl, and wherein up to eight hydrogen atoms are optionally substituted by methyl or a double bond, and the bond between carbons c and d may be unsaturated or saturated, or a pharmaceutically acceptable salt thereof.

### CROSS-REFERENCES TO RELATED APPLICATIONS

This application claims the benefit of U.S. Provisional Application No. 60/485,062 filed Jul. 2, 2003; and this application is also a continuation-in-part of U.S. patent application Ser. No. 10/112,509 filed Mar. 27, 2002 which claims benefit of both U.S. Provisional Application No. 60/336,289 filed Oct. 31, 2001 and U.S. Patent Application U.S. Patent Application 60/279,542 filed Mar. 27, 2001. The contents of which are each incorporated herein by reference in their entirety.

### STATEMENT AS TO RIGHTS TO INVENTIONS MADE UNDER FEDERALLY SPONSORED RESEARCH OR DEVELOPMENT

This invention was made with government support under Grant No. DA 12413, DA12447 and DA12653 awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

This invention relates to methods of screening compounds for OEA-like pharmacological activity and the use of such compounds, and compositions thereof, in the treatment of diseases or conditions mediated by PPARα or responsive to administration of PPARα modulators, including OEA.

### BACKGROUND OF THE INVENTION

Peroxisome proliferator activated receptors (PPAR) are a family of transcription factors and have been postulated to play a role in lipid homeostasis. Three PPAR subtypes have been identified: α, β (also described as δ), and γ. All three subtypes have domain structure common with other members of the nuclear receptor family. DNA binding domains are highly conserved among PPAR subtypes, but ligand binding domains are less well conserved. (Willson, et al. (2000) J. Med. Chem. 43:527). are highly conserved among PPAR subtypes, but ligand binding domains are less well conserved. (Willson, et al. (2000) J. Med. Chem. 43:527).

PPARs bind to RXR transcription factors to form heterodimers that bind to DNA sequences containing AGGTCAnAGGTCA. It has been shown that ligand binding to PPAR can induce gene expression.

PPARγ is the best characterized of the three subtypes. Activation of PPARγ promotes adipocyte differentiation by repressing expression of the ob and TNFα genes. Activation of PPARγ also results in in vivo insulin sensitization. PPARγ has been implicated in several diseases including diabetes, hypertension, dyslipidemia, inflammation, and cancer.

PPARα is expressed at high levels in the liver, heart, renal cortex, brown fat, and intestine. PPARα regulates genes involved in almost all aspects of lipid metabolism and has been postulated to play a role in dyslipidemia, atherosclerosis, obesity, and diabetes.

PPARβ(δ) is the most widely expressed subtype and the least understood. PPARβ(δ) regulates acyl-coA synthetase 2 expression and is postulated to play a role in dyslipidemia, fertility, bone formation, and colorectal cancer. PPARβ(δ) expression in cells reduces their proliferation rate, but PPARβ expression in cells in conjunction with exposure to fatty acids increases proliferation rate.

All three subtypes are postulated to play a role in lipid homeostasis, but comparative studies have demonstrated significant differences among the subtypes. For example, mRNA expression of PPARα and PPARα γ is increased in ob/ob and db/db mice, but mRNA expression of PPARβ(δ) in ob/ob and db/db mice is the same as in control mice. It has also been shown that some ligands that bind to PPARγ and PPARα do not bind or activate PPARβ(δ).

As stated above, the PPAR family has been described as playing a role in obesity. Natural and synthetic subtype specific ligands have been identified for PPARα, PPARα γ, and PPARβ(δ). PPARα-selective compounds have an enhanced ability to reduce body fat and modulate fatty acid oxidation compared to PPARβ or PPARγ selective compounds. PPARα is activated by a number of medium and long-chain fatty acids. PPARα is also activated by compounds known as fibric acid derivatives. These fibric acid derivatives, such as clofibrate, fenofibrate, bezafibrate, ciprofibrate, beclofibrate and etofibrate, as well as gemfibrozil reduce plasma triglycerides along with LDL cholesterol, and they are primarily used for the treatment of hypertriglyceridemia.

Fatty acid ethanolamides (FAE) are unusual components of animal and plant lipids, and their concentrations in non-stimulated cells are generally low (Bachur et al., J. Biol. Chem., 240:1019-1024 (1965); Schmid et al., Chem. Phys. Lipids, 80:133-142 (1996); Chapman, K. D., Chem. Phys. Lipids, 108:221-229 (2000)). FAE biosynthesis can be rapidly enhanced, however, in response to a wide variety of physiological and pathological stimuli, including exposure to fungal pathogens in tobacco cells (Chapman et al., Plant Physiol., 116:1163-1168 (1998)), activation of neurotransmitter receptors in rat brain neurons (Di Marzo et al., Nature, 372:686-691 (1994); Giuffrida et al., Nat. Neurosci., 2:358-363 (1999)) and exposure to metabolic stressors in mouse epidermal cells (Berdyshev et al., Biochem. J, 346:369-374 (2000)). The mechanism underlying stimulus-dependent FAE generation in mammalian tissues is thought to involve two concerted biochemical reactions: cleavage of the membrane phospholipid, N-acyl phosphatidylethanolamine (NAPE), catalyzed by an unknown phospholipase D; and NAPE synthesis, catalyzed by a calcium ion- and cyclic AMP-regulated N-acyltransferase (NAT) activity (Di Marzo et al., Nature, 372:686-691 (1994); Cadas et al., J. NeuroSci., 6:3934-3942 (1996); Cadas et al., H., J. Neurosci., 17:1226-1242 (1997)).

The fact that both plant and animal cells release FAEs in a stimulus-dependent manner suggests that these compounds may play important roles in cell-to-cell communication. Further support for this idea comes from the discovery that the polyunsaturated FAE, anandamide (arachidonylethanolamide), is an endogenous ligand for cannabinoid receptors (Devane et al., Science, 258:1946-1949 (1992))-G protein-coupled receptors expressed in neurons and immune cells, which recognize the marijuana constituent Δ⁹-tetrahydrocannabinol (Δ⁹-THC) (for review, see reference (Pertwee, R. G., Exp. Opin. Invest. Drugs, 9:1553-1571 (2000)).

Two observations make it unlikely that other FAEs also participate in cannabinoid neurotransmission. The FAE family is comprised for the most part of saturated and monounsaturated species, such as palmitoylethanolamide and oleoylethanolamide, which do not significantly interact with cannabinoid receptors (Devane et al., Science, 258:1946-1949 (1992); Griffin et al., J. Pharmacol. Exp. Ther., 292:886-894. (2000)). Second, when the pharmacological properties of the FAEs have been investigated in some detail, as is the case with palmitoylethanolamide, such properties have been found to differ from those of Δ⁹-THC and to be independent of activation of known cannabinoid receptor subtypes (Calignano et al., Nature, 394:277-281 (1998)). Thus, the biological significance of the FAEs remains elusive.

Oleoylethanolamide (OEA) is a natural analogue of the endogenous cannabinoid anandamide. Like anandamide, OEA is produced in cells in a stimulus-dependent manner and is rapidly eliminated by enzymatic hydrolysis, suggesting a role in cellular signaling However, unlike anandamide, OEA does not activate cannabinoid receptors and its biological mechanisms of action were here-to-fore essentially unknown.

Oleoylethanolamide is reported herein to be a potent and highly selective agonist of PPARα. With the discovery that OEA selectively modulates PPARα, the potential for using high throughput assays to identify other similar pharmacologically useful compounds which modulate PPARα is feasible. Such compounds will be useful in the treatment of PPARα-mediated diseases and conditions as well as any for which OEA was previously considered to be useful.

### BRIEF SUMMARY OF THE INVENTION

In a first aspect, the invention provides methods for identifying OEA-like compounds which are useful in the treatment of mammalian diseases or conditions mediated by PPARα or in the treatment of mammalian diseases or conditions mammalian diseases and conditions which are responsive to administration of a PPARα modulator (e.g., a PPARα agonist, a PPARα antagonist). The OEA-like compounds are identified by contacting the candidate compound with the PPARα receptor under assay conditions which measure the ability of the candidate compound to interact with the receptor (e.g., to occupy or bind to the receptor; to inhibit the receptor or inhibit the interaction of the receptor with another ligand; or by activating the receptor). In one set of embodiments, the contacting is in vitro. In another set of embodiments, the contacting is in vivo. In some embodiments, the methods are separately applied to a plurality of OEA-like compounds thereby screening such compounds for members having PPARα modulatory activity. In a further embodiment, the plurality is at least five or ten. In some embodiments, the disease or condition is obesity, an appetite disorder, overweight, a metabolic disorder, cellulite, Type II diabetes, insulin resistance, hyperlipidemia, hypercholesterolemia, hypertriglyceridemia, artherogenesis, an inflammatory disorder or condition, Alzheimers disease, Crohn's disease, vascular inflammation, an inflammatory bowel disorder, rheumatoid arthritis, thrombosis, asthma or cachexia.

In a second aspect, the invention provides methods for identifying OEA like compounds (e.g., fatty acid alkanolamide compounds and their homologs or analogs) and OEA-like modulators which are useful in the treatment of mammalian diseases or conditions mediated by PPARα or mammalian diseases and conditions which are responsive to administration of a PPARα modulator. In one set of embodiments, the candidate compound modulator is a PPARα antagonist (e.g., a compound which can inhibit, block, or occupy the PPARα receptor without activating it). In another set of embodiments, the compound modulator is a PPARα agonist (e.g., a compound which can activate or transduce PPARα receptor upon binding). In one set of embodiments, the contacting is in vitro. In another set of embodiments, the contacting is in vivo. In some embodiments, the methods are separately applied to a plurality of OEA-like compounds thereby screening such compounds for members having PPARα modulatory activity. In a further embodiment, the plurality is at least five or ten. In some embodiments, the disease or condition is obesity, an appetite disorder, overweight, a metabolic disorder, cellulite, Type II diabetes, insulin resistance, hyperlipidemia, hypercholesterolemia, hypertriglyceridemia, artherogenesis, an inflammatory disorder or condition, Alzheimers disease, Crohn's disease, vascular inflammation, an inflammatory bowel disorder, rheumatoid arthritis, asthma, thrombosis or cachexia. In some embodiments, the OEA-like compound is a compound of Formula I or Formula VI. In embodiments, the above identified compounds are used in the treatment of the above diseases.

In another aspect, the invention provides pharmaceutical compositions comprising an OEA-like modulator of PPARα and methods of treating a disease or condition which is mediated by PPARα or therapeutically responsive to administration by a modulator of PPARα by administering an OEA-like modulator to a host or subject having the condition. The disease or condition to be treated includes, but are not limited to, metabolic disorders, obesity, excess body fat, cellulite, Type II diabetes, insulin resistance, hyperlipidemia, hypercholesterolemia, hypertriglyceridemia, artherogenesis, an inflammatory disorder or condition, Alzheimers disease, Crohn's disease, a vascular inflammation, an inflammatory bowel disorder, rheumatoid arthritis, asthma, thrombosis or cachexia. In some embodiments, the subject is a mammal, including, but not limited to, humans, rats, mice, rabbits, dogs, cats, hamsters, and primates.

In another aspect, the OEA-like compound is an antagonist of PPARα and the disease or condition includes, but is not limited to, loss of appetite, low body weight (e.g, a BMI of less than 18.5 in an adult), anorexia, anorexia nervosa, cancer cachexia, and AIDS-dependent wasting syndrome. In some embodiments, the compound is a selective inhibitor or antagonist of PPARα. In other embodiments, the antagonist is a fatty acid alkanolamide, or a homolog or analog thereof. In some some embodiments, the alkanolamide is a compound of Formula I or Formula VI.

In still another aspect, the present invention provides a method of identifying a compound for reducing body fat in an animal by testing the compound for its activity as a specific agonist of peroxisome proliferator activated receptor type a (PPARα) in a PPAR modulation assay panel comprising PPARα, PPARγ and PPARβ. A specific agonist of peroxisome proliferator activated receptor type a (PPARα) is identified by testing the compound in activation assays for each of PPARα, PPARγ and PPARβ and selecting the compound which has at least a 5 fold specificity for PPARα over either or both of PPARγ and PPARβ under comparable or physiological assay conditions. The identified PPARα selective compound can then be tested in an animal model by administering the compound to a subject and determining body fat reduction in the subject. In some embodiments, the specificity for PPARα over either or both of PPARγ and PPARβ is at least five-fold, ten-fold, twenty-fold or one hundred-fold. In some embodiments, the PPARα selective agonist has a half maximal effect at a concentration less than 1 micromolar, 100 nanomolar or 1 nanomolar, or between 1 micromolar and 10 nanomolar. In some embodiments, the compound is OEA-like compound, including but not limited to, fatty acid alkanolamides. In some embodiments, the subject is a mammal. In some further embodiments, the subject is a human, mouse, rat, rabbit, hamster, guinea pig or primate.

Preferred embodiments for identifying candidate compounds in vitro include measuring expression of reporter genes or proliferation of PPARα transfected cells. Preferred embodiments for measurements of body fat reduction in mammals include measuring changes in the body weight of the mammal or using calipers to measure body fat. Preferred mammals include, but are not limited to, mice, rats, guinea pigs, or rabbits, ob/ob mice, db/db mice, or Zucker rats.

In still another aspect, the present invention is a method of identifying a compound for modulating fatty acid metabolism. A specific agonist of peroxisome proliferator activated receptor type α (PPARα) is identified by testing the compound in activation assays for each of PPARα, PPARγ and PPARβ and selecting the compound which has at least a 5 fold specificity for PPARα over PPARγ and PPARβ. The selected or candidate compound can then be tested in an animal model by administering the compound to a subject and determining body fat reduction in the subject. In some embodiments, the specificity for PPARα over each of PPARγ and PPARβ is at least five-fold, ten-fold, twenty-fold or one-hundred fold. In some embodiments, the PPARα selective agonist has a half maximal effect at a concentration less than 1 micromolar, 100 nanomolar or 1 nanomolar, or between 1 micromolar and 10 nanomolar. In some embodiments, the compound is an OEA-like compound, including but not limited to, a fatty acid alkanolamide. In some embodiments, the subject is a mammal. In some further embodiments, the subject is a human, mouse, rat, rabbit, hamster, guinea pig or primate.

In still another aspect, the present invention is a method of identifying a compound for modulating appetite or treating an appetite disorder. A specific agonist of peroxisome proliferator activated receptor type α (PPARα) is identified by testing the compound in activation assays for each of PPARα, PPARγ and PPARβ and selecting the compound which has at least a 5 fold specificity for PPARα over PPARγ and PPARβ. The selected or candidate compound can then be tested in an animal model by administering the compound to a subject and determining the effect of the administration on body fat, body weight, or food consumption, for example, by comparison of such measures for an appropriate control population. In some embodiments, the specificity for PPARα over each of PPARγ and PPARβ is at least five-fold, ten-fold, twenty-fold or one-hundred fold. In some embodiments, the PPARα selective agonist has a half maximal effect at a concentration less than 1 micromolar, 100 nanomolar or 1 nanomolar, or between 1 micromolar and 10 nanomolar. In some embodiments, the compound is OEA-like compound, including but not limited to, a fatty acid alkanolamide. In some embodiments, the subject is a mammal. In some further embodiments, the subject is a human, mouse, rat, rabbit, hamster, guinea pig or primate. In some embodiments, the disease or condition to be treated is an appetitive disorder, including, but not limited to, bulimia.

Preferred embodiments for measuring the interaction of a compound including, but not limited to, OEA-like compounds and fatty acid alkanolamide compounds, with a PPARa receptor in vitro measure the expression of reporter genes or proliferation of PPARα transfected cells.

Preferred embodiments for measurements of body fat reduction in mammals include measuring changes in the body weight of the mammal or using calipers to measure body fat.

Preferred embodiments for measurement modulation of fatty acid metabolism also include measuring lipolysis in adipocytes, fatty acid oxidation in hepatocytes and myocytes and measuring body mass or body fat in the animal.

In preferred embodiments, the PPARα modulator or OEA-like compound is a compound having the formula: 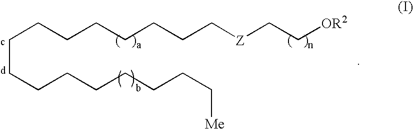
or its pharmaceutically acceptable salt.

In this formula, n is from 0 to 5 and the sum of a and b can be from 0 to 4. Z is a member selected from —C(O)N(R^{o})—; —(R^{o})NC(O)—; —OC(O)—; —(O)CO—; O; NR^{o}; and S, in which R¹ and R² are independently selected from the group consisting of unsubstituted or unsubstituted alkyl, hydrogen, substituted or unsubstituted C₁-C₆ alkyl, substituted or unsubstituted lower (C₁-C₆) acyl, homoalkyl, and aryl. Up to eight hydrogen atoms of the compound may also be substituted by methyl or a double bond. In addition, the molecular bond between carbons c and d may be unsaturated or saturated.

The present invention also provides compounds, compositions, and methods for modulating appetite or treating an appetency disorder, reducing body fat and for treating or preventing obesity, and overweight in mammals and the diseases associated with these health conditions. In one aspect, the invention provides methods for reducing body fat or body weight and for treating or preventing obesity or overweight and for reducing food intake by administration of pharmaceutical compositions comprising an OEA-like compound in an amount sufficient to reduce body fat, body weight or prevent body fat or body weight gain. In other aspects, the invention is drawn to the fatty acid ethanolamide compounds, homologues, analogs; and their pharmaceutical compositions and such methods of use.

In other embodiments, the fatty acid moiety of the fatty acid alkanolamide or ethanolamide compound, homologue, or analog may be saturated or unsaturated, and if unsaturated may be monounsaturated or polyunsaturated.

In some embodiments, the fatty acid moiety of the fatty acid alkanolamide compound, homologue, or analog is a fatty acid selected from the group consisting of oleic acid, palmitic acid, elaidic acid, palmitoleic acid, linoleic acid, α-linolenic acid, and γ-linolenic acid. In certain embodiments, the fatty acid moieties have from twelve to 20 carbon atoms.

Other embodiments are provided by varying the hydroxyalkylamide moiety of the fatty acid amide compound, homologue or analog. These embodiments include the introduction of a substituted or unsubstituted lower (C₁-C₃) alkyl group on the hydroxyl group of an alkanolamide or ethanolamide moiety so as to form the corresponding lower alkyl ether. In another embodiment, the hydroxy group of the alkanolamide or ethanolamide moiety is bound to a carboxylate group of a C₂ to C₆ substituted or unsubstituted alkyl carboxylic acid to form the corresponding ester of the fatty acid ethanolamide. Such embodiments include fatty acid alkanolamide and fatty acid ethanolamides in ester linkage to organic carboxylic acids such as acetic acid, propionic acid, and butanoic acid. In one embodiment, the fatty acid alkanolamide is oleoylalkanolamide. In a further embodiment, the fatty acid alkanolamide is oleoylethanolamide.

In still another embodiment, the fatty acid ethanolamide compound, homologue, or analog further comprises a substituted or unsubstituted lower alkyl (C₁-C₃) group covalently bound to the nitrogen atom of the fatty acid ethanolamide.

In other aspects of the invention, the methods and compositions employ fatty acid ethanolamide and fatty acid alkanolamide compounds, homologs and analogs for reducing body weight in which the compounds, homologs and analogs cause weight loss when administered to test animals (e.g., rats, mice, rabbits, hamsters, guinea pigs).

In still other aspects, a preferred compound of the invention is a fatty acid alkanolamide, or homologs and analogs, thereof, which is a selective agonist of the PPARα receptor. Preferred compounds include, but are not limited to, a fatty acid alkanolamide or compound of formula I which provides a half-maximal modulatory effect on the PPARα receptor at a concentration which is at least 5-fold, 10-fold, 50-fold, or 100-fold lower than the concentration of the compound which provides a half-maximal effect (or no effect) on a PPARβ or PPARγ receptor from the same species of origin as the PPARα receptor under comparable assay conditions (e.g., same in vivo test species and conditions or same pH, same buffer components). Still further preferred PPARα-agonist compounds, including OEA-like compounds, have a half maximal modulatory effect on the receptor at a concentration of less than 1 micromolar, less than 100 nanomolar, and more preferably less than 10 nanomolar.

Still other aspects of the invention address methods of using and administering selective high affinity (high affinity indicates an ability to produce a half-maximal effect at a concentration of 1 micromolar or less) agonists of PPARα for reducing body weight or reducing appetite or reducing food intake or causing hypophagia in mammals (e.g., humans, primates, cats, dogs). The subject compositions may be administered by a variety of routes, including orally. In some embodiments, the selective high affinity agonists of PPARα are OEA-like compounds, including, but not limited to, fatty acid alkanolamides and the compounds according to Formula I above and Formula VI below.

In still other aspects of the invention, a Fatty Acid Amide Hydrolase (FAAH) inhibitor is administered to treat a condition or disease in a subject mediated by PPARα or responsive to therapy with a PPARα agonist. In some embodiments, the PPARα agonist is an OEA-like compound, including, but not limited to a compound of Formula I or Formula VI. In some further embodiments, the FAAH inhibitor is administered to a subject also receiving a PPARα agonist, including but not limited to an agonist of Formula I, Formula VI, and particularly, selective PPARα agonists. In preferred embodiments, the subject is human. In some embodiments, the OEA-like modulator is an agonist of PPARα and the disease or condition to be treated is a metabolic disorder, obesity, excess body fat, cellulite, type II diabetes, insulin resistance, hyperlipidemia, hypercholesterolemia, hypertriglyceridemia, artherogenesis, an inflammatory disorder or condition, Alzheimers disease, Crohn's disease, a vascular inflammation, an inflammatory bowel disorder, an immune disorder, autoimmunity, environmental immunity, rheumatoid arthritis, asthma, or thrombosis. The FAAH inhibitor may work by inhibiting the FAAH-mediated hydrolysis of an administered OEA like compound subject to such hydrolysis and/or by inhibiting the hydrolysis of endogenously formed OEA or another endogenous FAAH substrate. In a preferred embodiment, the FAAH inhibitor is administered with a OEA-like compound subject to hydrolysis by FAAH so that the biological half-life of the OEA like compound is increased. In one embodiment, the co-administered OEA like compound is OEA.

In a further aspect, the invention provides a cell line for testing OEA-like compound such as fatty acid alkanolamides for their ability to bind to or transduce PPAR. The cell line is one which essentially lacks the ability to enzymatically hydrolyze OEA and which stably expresses an exogenous reporter gene and a PPAR or RXR receptor gene operably linked to a regulatory domain. In some embodiments, the PPAR is PPARα. The fatty acid alkanolamide is contacted with such a cell line and any subsequent transduction of the PPAR is determined by detecting the expression of the reporter gene.

In still another aspect, the invention provides OEA-like compounds, compositions, and methods for their use in treating diseases and conditions mediated by PPARα or responsive to PPARα agonists. Such compounds include, in particular, OEA and fatty acid alkanolamides and homologs and compounds of formula I or formula VI. In some embodiments, the disease or condition is obesity, an appetite disorder, overweight, a metabolic disorder, cellulite, Type II diabetes, insulin resistance, hyperlipidemia, hypercholesterolemia, hypertriglyceridemia, artherogenesis, an inflammatory disorder or condition, Alzheimers disease, Crohn's disease, vascular inflammation, an inflammatory bowel disorder, rheumatoid arthritis, asthma, or thrombosis.

### BRIEF DESCRIPTION OF THE DRAWINGS

FIG. 1. Starvation increases circulating oleoylethanolamide levels in rats: (a) time course of the effects of food deprivation on plasma oleoylethanolamide (OEA) levels; (b) effect of water deprivation (18 h) on plasma oleoylethanolamide levels; (c) effect of food deprivation (18 h) on oleoylethanolamide levels in cerebrospinal fluid (CSF); (d) time course of the effects of food deprivation on plasma anandamide (arachidonylethanolamide, AEA) levels; (e) effect of water deprivation (18 h) on anandamide plasma levels; (f) effect of food deprivation (18 h) on anandamide levels in CSF. Results are expressed as mean±s.e.m.; asterisk, P<0.05; two asterisks, P<0.01, n=10 per group.

FIG. 2. Adipose tissue is a primary source of circulating oleoylethanolamide: starvation-induced changes in N-acyltransferase (NAT) and fatty acid amide hydrolase (FAAH) activities in various rat tissues. (a) fat; (b) brain; (c) liver; (d) stomach; (e) small intestine. Empty bars, free-feeding animals; filled bars, 18-h fasted animals. Activities are in pmol/mg protein/min. Asterisk, P<0.05, n=3.

FIG. 3. Adipose tissue is a primary source of circulating oleoylethanolamide: starvation-induced changes in NAPE and oleoylethanolamide (oleoylethanolamide, OEA) content in adipose and liver tissues. (a) structures of the oleoylethanolamide precursors alk-1-palmitoenyl-2-arachidonyl-sn-glycero-phosphoethanolamine-N-oleyl (left panel, NAPE 1) and alk-1-palmityl-2-arachidonyl-sn-glycero-phosphoethanolamine-N-oleyl (right panel, NAPE 2); (b) representative HPLC/MS tracings for selected ions characteristic of NAPE 1 (left panel, m/z=987, deprotonated molecule, [M−H]⁻) and NAPE 2 (right panel, m/z=1003, [M−H]⁻) in free-feeding (top) and 18-h fasting rats (bottom); (c) food deprivation (18 h) increases the content of NAPE species in fat and decreases it in liver. All identifiable NAPE species were quantified, including the oleoylethanolamide precursors NAPE1 and NAPE 2, and the PEA precursor NAPE 3; (d) food deprivation (18 h) increases oleoylethanolamide content in fat and liver. Empty bars, free-feeding animals; filled bars, 18-h fasted animals. Asterisk, P<0.05, Student's t test; n=3.

FIG. 4. Oleoylethanolamide (OEA/pranamide) selectively suppresses food intake: (a) dose-dependent effects of oleoylethanolamide (i.p., empty squares), elaidylethanolamide (empty circles), PEA (triangles), oleic acid (filled squares) and anandamide (filled circles) on food intake in 24-h food-deprived rats. Vehicle alone (70% DMSO in saline, 1 ml per kg, i.p.) had no significant effect on acute food intake; (b) time course of the hypophagic effects of oleoylethanolamide (20 mg per kg, i.p.) (squares) or vehicle (lozenges) on food intake. (c) effects of vehicle (V), lithium chloride (LiCl, 0.4 M, 7.5 ml per kg) or oleoylethanolamide (20 mg per kg) in a conditioned taste aversion assay. Empty bars, water intake; filled bars, saccharin intake. Effects of vehicle (V) or oleoylethanolamide (5 or 20 mg per kg) on: (d) water intake (expressed in ml per 4 h); (e) body temperature; (f) latency to jump in the hot plate analgesia test; (g) percent time spent in open arms in the elevated plus maze anxiety test; (h) number of crossings in the open field activity test; (i) number of operant responses for food. Asterisk, P<0.05, n=8-12 per group.

FIG. 5. Effects of subchronic oleoylethanolamide administration on food intake and body weight: (a) effects of oleoylethanolamide (OEA) (5 mg per kg, i.p. once a day) (empty bars) or vehicle (5% Tween 80/5% polyethyleneglycol in sterile saline; filled bars) on cumulative food intake; (b) time course of the effects of oleoylethanolamide (triangles) or vehicle (squares) on body weight change; (c) effects of oleoylethanolamide or vehicle on net body weight change; (d) effects of oleoylethanolamide (5 mg per kg) or vehicle on cumulative water intake. Asterisk, P<0.05; two asterisks, P<0.01, n=10 per group.

FIG. 6. Role of peripheral sensory fibers in oleoylethanolamide-induced anorexia. Effects of vehicle (V), oleoylethanolamide (oleoylethanolamide/pranamide/OEA) (5 mg per kg, i.p.), CCK-8 (10 μg per kg) and CP-93129 (1 mg per kg), a centrally active 5-HT_{1B} receptor agonist, on food intake in a, control rats and c, capsaicin-treated rats. Water intake in (b) control rats and (d) capsaicin-treated rats. Asterisk, P<0.05; n=8-12 per group.

FIG. 7. Oleoylethanolamide increases c-fos mRNA expression in discrete brain regions associated with energy homeostasis and feeding behavior: (a) pseudocolor images of film autoradiographs show that oleoylethanolamide (right section) elicits a striking and selective increase in c-fos mRNA labeling in the paraventricular (PVN) and supraoptic (SO) hypothalamic nuclei, as assessed by in situ hybridization. A representative section from a vehicle-treated rat is shown at left. Labeling densities are indicated by color: blue<green<yellow<red. (b) quantification of c-fos cRNA labeling in forebrain regions [PVN, SO, arcuate (Arc), layer II piriform cortex (pir), ventrolateral thalamas (VI) and S1 forelimb cortex (S1FL)] of rats treated with vehicle, oleoylethanolamide and oleic acid; (c) film autoradiogram showing elevated ³⁵S c-fos mRNA expression in the nucleus of the solitary tract (NST) in an oleoylethanolamide-treated rat; Inset, c-fos cRNA labeling in the NST (shown in red) was identified by its localization relative to adjacent efferent nuclei (hypoglossal and dorsal motor nucleus of the vagus), which express choline acetyl transferase (ChAT) mRNA (shown in purple); (d) oleoylethanolamide increases c-fos mRNA expression in NST but not in the hypoglossal nucleus (HgN). Two asterisks, P<0.0001, n=5 per group.

FIG. 8. The effects of OEA, Oleic acid (OA), AEA, PEA, and methyl-OEA on fatty acid oxidation in soleus muscle.

FIG. 9. Activation of human PPARα-GAL4 chimeric receptors by OEA. a, Concentration-dependent effects of OEA on PPARα (closed circles), PPARδ (open triangles), PPARγ (closed squares) and RXR (open lozenges). b, Effects of OEA (closed circles), oleic acid (open squares), stearylethanolamide (closed triangles), myristylethanolamide (closed squares), and anandamide (open circles) on PPARα activation. Results are the mean±s.e.m. of n=16.

FIG. 10. OEA reduces feeding in wild-type mice, but not in mice deficient for PPAR-α. Time course of the hypophagic effects of OEA (10 mg-kg⁻¹, i.p.) (closed squares) or vehicle (70% DMSO in saline, 1 ml-kg-1, i.p)(open squares) on cumulative food intake normalized for body weight in a, wild-type mice, and b, PPAR-α-null mice. c, Effects of vehicle (V), d-fenfluramine (4 mg-kg⁻¹, i.p.) or cholecystokinin-octapeptide (25 μg-kg⁻¹, i.p.) on cumulative food intake in wild-type (+/+) and PPAR-α-null (−/−) mice. Asterisk, P<0.05; n=8-12 per group.

FIG. 11. Subchronic OEA administration reduces food intake and body mass in wild-type, but not in PPAR-α null mice. Effects of OEA (5 mg mg-kg⁻¹, i.p.) (solid bars) or vehicle (propylenglycol/Tween80/saline, May 5, 1990; 1 ml-kg⁻¹, i.p) (open bars) on a, cumulative food intake normalized for body weight; b, cumulative body-weight gain; c, liver tissue triglycerides; d, white adipose tissue triglycerides; and e, serum cholesterol, in wild-type (+/+) and PPAR-α-null (−/−) mice. Asterisk, P<0.05; Two asterisks, P<0.001; n=10 per group.

FIG. 12. Synthetic PPAR-α agonists mimic the satiety-inducing actions of OEA. a, Effects of vehicle (open squares), Wy-14643 (closed triangles) (40 mg kg⁻¹, i.p.) and GW-7647 (open circles) (20 mg kg⁻¹, i.p.) on cumulative food intake normalized for body weight in C57BL/6J mice (vehicle, n=40; drugs, n=4-7). b, Effects of vehicle (V, open bars), Wy-14643 (W) (40 mg kg⁻¹, i.p.), GW-7647 (G₁) (20 mg kg⁻¹, i.p.) and OEA (O) (10 mg kg⁻¹, i.p.) on feeding latency, first meal size (MS) and first post-meal interval (PMI) in C57BL/6J mice (vehicle, n=40; drugs, n=4-7). c, Effects of vehicle (V, open bars), OEA (O) (10 mg kg⁻¹, i.p.) and d-fenfluramine (F) (3 mg kg⁻¹, s.c.) on food intake in control rats (sham, n=5-8) and vagotomized rats (vag, n=5-6). d-e, Time-course of the effects of vehicle (open symbols) or Wy-14643 (closed symbols) (40 mg kg⁻¹, i.p.) on food intake in d, control rats (n=7-8) and e, vagotomized rats (n=5-6). f, Lack of effect of the PPAR-β/δ agonist GW501516 (G₂) (5 mg kg⁻¹, i.p.) and PPAR-y agonist ciglitazone (C) (15 mg kg⁻¹, i.p.) on cumulative food intake in C57BL/6J mice (vehicle, n=40; drugs, n=4-6 per group). g-h, Time-course of the effects of vehicle (open symbols) or Wy-14643 (closed symbols) (40 mg kg⁻¹, i.p.) on cumulative food intake normalized for body weight in g, wild-type mice (n=8-11) and h, PPAR-α null mice (n=7-8). Asterisk, P<0.05; two asterisks, P<0.001; three asterisks, P<0.0001; one-way ANOVA followed by Dunnett's test or, when appropriate, t-test with Bonferroni's correction.

FIG. 13. OEA regulates gene expression in the jejunum and liver of wild-type but not PPAR-α null mice. a-g, Activation of gene expression by OEA in a-d, jejunum; e-g, liver. a-e, Effects of vehicle (V, open bar), Wy-14643 (W) (30 mg kg-1, i.p.) or OEA (O) (10 mg kg⁻¹, i.p.) on mRNA levels of a, PPAR-α; b, FAT/CD36; c, FATP1; and d, PPAR-8, PPAR-γ and I-FABP in the jejunum of wild-type (+/+) and PPAR-A null (−/−) mice (n=5 per group). e-g, Effects of vehicle (V, open bars), Wy-14643 (W) (30 mg kg⁻¹, i.p.) or OEA (O) (10 mg kg-1, i.p.) on mRNA levels of e, PPAR-α; f, FAT/CD36; and g, liver-FABP in wild-type (+/+) and PPAR-α null (−/−) mice (n=5 per group). h, Transrepression of iNOS expression by OEA (O) (10 mg kg⁻¹, i.p.) and Wy-14643 (W) (30 mg kg⁻¹, i.p.) in the jejunum of C57BL/6J mice (n=5). mRNA levels are expressed in arbitrary units. Asterisk, P<0.05; two asterisks, P<0.001; one-way ANOVA followed by Dunnett's test.

FIG. 14. OEA initiates expression of PPAR-α-regulated genes in the duodenum of wild-type but not PPAR-α-null mice. a, Time course of the effects of vehicle (open bars) or OEA (solid bars) (10 mg kg⁻¹, i.p.) on PPAR-α mRNA levels in the duodenum of C57BL/6J mice (n=5 per group). b-e, Effects of vehicle (V, open bar), Wy-14643 (W) (30 mg kg⁻¹, i.p.) or OEA (O) (10 mg kg⁻¹, i.p.) on mRNA levels of b, PPAR-α; c, FAT/CD36; d, FATP1; and e, PPAR-δ, PPAR-γ and I-FABP in wild-type (+/+) and PPAR-α-null (−/−) mice (n=5 per group). mRNA levels were measured as described under Methods and are expressed in arbitrary units. Asterisk, P<0.05; two asterisks, P<0.001.

FIG. 15. OEA and synthetic PPAR-α agonists fail to induce expression of PPAR-α-regulated genes in the ileum of wild-type and PPAR-α-null mice. Effects of vehicle (V, open bars), Wy-14643 (W) (30 mg kg⁻¹, i.p.) or OEA (O) (10 mg kg⁻¹, i.p.) on mRNA levels of a, PPAR-α; b, FAT/CD36; c, FATP1; and d, PPAR-δ, PPAR-γ and I-FABP in wild-type (+/+) and PPAR-α-null (−/−) mice (n=5 per group). mRNA levels were measured as described under Methods and are expressed in arbitrary units. Asterisk, P<0.05; two asterisks, P<0.001.

FIG. 16. Concerted regulation of intestinal OEA synthesis and PPAR-α expression. a, Food intake; b, OEA content; c, PPAR-α mRNA levels; and d, iNOS mRNA levels at night-time (1:30 AM; closed bars) and daytime (4:30 PM; open bars) in free-feeding C57B1/6J mice maintained on a 12:12 dark/light cycle (n=3). Asterisk, P<0.05; two asterisks, P<0.001; Student's t-test.

FIG. 17. Effect of subhronic OEA administration (5 mg/kg, once daily for 2 weeks, i.p.) on food intake and body weight gain over the two week period. Black circles, OEA. Open squares, vehicle.

### DETAILED DESCRIPTION OF THE INVENTION

It has been advantageously discovered that:

(1) OEA selectively engages with high affinity the peroxisome proliferator-activating receptor alpha (PPARα), a ligand-operated transcription factor that regulates multiple aspects of lipid metabolism.

(2) Administration of OEA produces satiety and reduces body-weight gain in wild-type mice, but not in mice deficient in PPARα.

(3) Two structurally distinct, high-affinity PPARα agonists exert similar effects, which also are contingent on PPARα expression; and that, in contrast, potent and selective agonists for PPARγ and PPARδ are ineffective.

(4) In the small intestine and liver of wild-type, but not PPARα null mice, OEA initiates transcription of several PPARα regulated genes, including those encoding for the fatty acid transporters FATP1 and FAT/CD36.

The above findings indicate that OEA induces satiety by acting as a high-affinity ligand for PPARα and suggest a role for OEA signaling via PPARα in the regulation of lipid metabolism. The results further indicate the importance of PPARα in the mediation of diseases and conditions related to body fat burden, obesity, metabolic disorders, and appetite. The results further show that OEA-like compounds, including but not limited to, fatty acid alkanolamides and homologs thereof can be potent and selective PPARα modulators. Such modulators find use in the treatment of diseases and conditions mediated by PPARα (e.g., diseases responsive to administration of agonists of PPARα). The results further indicate the high affinity specific PPARα agonists or OEA-like modulators are particularly useful in the treatment of appetite disorders, obesity, and in reducing body fat and body weight.

### Definitions

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

Where substituent groups are specified by their conventional chemical formulae, written from left to right, they equally encompass the chemically identical substituents which would result from writing the structure from right to left, e.g., —CH₂O— is intended to also recite —OCH₂—.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. The term “pharmaceutical composition” indicates a composition suitable for pharmaceutical use in a subject, including an animal or human. A pharmaceutical composition generally comprises an effective amount of an active agent and a pharmaceutically acceptable carrier.

Compounds of the invention may contain one or more asymmetric centers and can thus occur as racemates and racemic mixtures, single enantiomers, diastereomeric mixtures and individual diastereomers. The present invention is meant to comprehend all such isomeric forms of the inventive compounds.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed by the inventive formulas.

Compounds of the invention include the diastereoisomers of pairs of enantiomers. Diastereomers for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of an inventive compound may be obtained by stereospecific synthesis using optically pure starting materials or reagents of known configuration

As used herein, the term “heteroatom” is meant to include oxygen (O), nitrogen (N), sulfur (S) and silicon (Si).

“Alkanol,” as used herein, refers to a saturated or unsaturated, substituted or unsubstituted, branched or unbranched alkyl group having a hydroxyl substituent, or a substituent derivable from a hydroxyl moiety, e.g,. ether, ester. The alkanol is preferably also substituted with a nitrogen-, sulfur-, or oxygen-bearing substituent that is included in bond Z (Formula I), between the “fatty acid” and the alkanol.

“Fatty acid,” as used herein, refers to a saturated or unsaturated substituted or unsubstituted, branched or unbranched alkyl group having a carboxyl substituent. Preferred fatty acids are C₄-C₂₂ acids. Fatty acid also encompasses species in which the carboxyl substituent is replaced with a —CH₂— moiety.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CHi)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, =O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′-C(O)NR″R′″, —NR″C(O)₂R′, —NR—C(NR′R″R′″)═NR″″, —NR—C(NR′R″)=NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′-C(O)NR″R′″, —NR″C(O)₂R′, —NR—C(NR′R″ R′″)=NR“ ”, —NR—C(NR′R″)=NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

The term “body fat reduction” means loss of a portion of body fat.

The formula for Body Mass Index (BMI) is [Weight in pounds. Height in inches Height in inches]×703. BMI cutpoints for human adults are one fixed number, regardless of age or sex, using the following guidelines: Overweight human adults individuals have a BMI of 25.0 to 29.9. Obese human adults have a BMI of 30.0 or more. Underweight adults have a BMI less of than 18.5. A nomal body weight range for an adult is defined as a BMI between 18.5 and 25. BMI cutpoints for children under 16 are defined according to percentiles: Overweight is defined as a BMI for age greater than ≧85th percentile and obesity is defined as a BMI-for-age ≧95th percentile. Underweight is a BMI-for-age <5th percentile. A normal body weight range for a child is defined as a BMI above the 5th percentile and below the 85 percentile. In some embodiments, the OEA-like compounds of the invention are used to treat obesity and/or overweight. In some embodiments, PPARt antagonists are used to treat underweight.

The term “fatty acid oxidation” relates to the conversion of fatty acids (e.g., oleate) into ketone bodies.

Fatty acid amide hydrolase is the enzyme primarily responsible for the hydrolysis of anandamide in vivo. It also is responsible for the hydrolysis of OEA in vivo. Inhibitors of the enzyme are well known to one of ordinary skill in the art.

The term “hepatocytes” refers to cells originally derived from liver tissue. Hepatocytes may be freshly isolated from liver tissue or established cell lines.

The term “modulate” means to induce any change including increasing or decreasing. (e.g., a modulator of fatty acid oxidation increases or decreases the rate of fatty oxidation. A modulator of a receptor includes both agonists and antagonists of the receptor.

The term “muscle cells” refers to cells derived from the predominant cells of muscle tissue. Muscle cells may be freshly isolated from muscle tissue or established cell lines.

Oleoylethanolamide (OEA) refers to a natural lipid of the following structure:

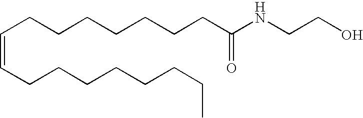

An OEA-like compound includes, but is not limited to, fatty acid alkanolamides, fatty acid ethanolamide compounds, and their analogs and homologues which modulate the PPARα receptor. Exemplary OEA-like compounds are compounds of formula I or Formula VI which modulate the PPARα receptor. OEA-like compounds include agonists and antagonists of the PPARα receptor. OEA-like compounds which selectively modulate the PPARα receptor are preferred. Particularly preferred OEA-like modulators have a selective affinity of at least 10-fold, 50-fold or 100-fold greater for PPARα than for PPARβ or PPARγ. Such preferred OEA-like compound are particularly preferred if they produce a half-maximal effect on the PPARα receptor under physiological conditions at a concentration of 1 micromolar or less, 100 nanomolar or less, 10 nanomolar or less, or 1 nanomolar or less, or from 1 micromolar to 1.0 nanomolar, or less. Such OEA-like compounds can include, but are not limited to, fatty acid alkanolamides, their homologues and analogues. Particularly preferred OEA-like compounds are also selective for the PPARα receptor over a cannabinoid receptor. Such compounds include those compounds whose affinity for the PPARα receptor is at least 5-fold, 10-fold, or 50-fold greater than that for a cannabinoid receptor (e.g., CB₁ or CB₂ receptor). OEA is an example of a preferred OEA-like compound.

An OEA-like modulator or OEA like agonist is a PPARα agonist having a selective affinity for the PPARα receptor at least 5-fold greater (e.g., having a concentration which produces a half-maximal effect which is at least 5-fold lower) than for either or both PPARβ or PPARγ as measured under comparable bioassay conditions in vivo or in vitro or in any bioassay as described herein. Particularly preferred OEA-like modulators have a selective affinity of at least 5-fold, 10-fold, 50-fold or 100-fold greater for PPARα than for PPARβ, or PPARγ. Such preferred OEA-like compounds are particularly preferred if they produce a half-maximal effect on the PPARα receptor under physiological conditions at a concentration of 1 micromolar or less, 100 nanomolar or less, 10 nanomolar or less, or 1 nanomolar or less, or from 1 micromolar to 1.0 nanomolar, or less. Such OEA-like compounds can include, but are not limited to, fatty acid alkanolamides, their homologues and their analogues. Also particularlyt preferred are OEA and compounds of Formula I or Formula VI. In other embodiments, the OEA-like modulator is a specific high affinity agonist of PPARα which is not a fatty acid alkanolamide or a homolog thereof and is not a compound of Formula I or Formula VI. Particularly preferred OEA-like modulators are selective for the PPARa receptor over a cannabinoid receptor. Such modulators include compounds whose affinity for the PPARα receptor is at least 5-fold, 10-fold, or 50-fold greater than that for a cannabinoid receptor (e.g., CB₁ or CB₂ receptor).

In the formulas herein, “Me” represents the methyl group.

The term “weight loss” refers to loss of a portion of total body weight.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995). Preferred pharmaceutical carriers depend upon the intended mode of administration of the active agent. Typical modes of administration are described below.

The term “effective amount” means a dosage sufficient to produce a desired result on health. The desired result may comprise a subjective or objective improvement in the recipient of the dosage. A subjective improvement may be, for instance, decreased appetite or craving for food. An objective improvement may be, for instance, decreased body weight, body fat, or food, decreased food consumption, decreased food seeking behavior, or improved serum lipid profile, or a decreased likelihood of developing a disease or harmful health condition.

A “prophylactic treatment” is a treatment administered to a subject who does not exhibit signs of a disease or exhibits only early signs of a disease, wherein treatment is administered for the purpose of decreasing the risk of developing a pathology associated with the disease. The compounds of the invention may be given, for instance, as a prophylactic treatment to prevent undesirable or unwanted weight gain.

A “therapeutic treatment” is a treatment administered to a subject who exhibits signs or symptoms of pathology, wherein treatment is administered for the purpose of diminishing or eliminating those pathological signs.

“Diseases or conditions mediated by PPARα or responsive to administration of a PPARα modulator” include, but are not limited to, each of obesity, an appetite disorder, overweight, a metabolic disorder, cellulite, Type I and Type II diabetes, hyperglycemia, dyslipidemia, Syndrome X, insulin resistance, diabetic dyslipidemia, anorexia, bulimia, anorexia nervosa, hyperlipidemia, hypercholesterolemia, hypertriglyceridemia, artherogenesis, artherosclerosis, an inflammatory disorder or condition, Alzheimers disease, Crohn's disease, vascular inflammation, an inflammatory bowel disorder, rheumatoid arthritis, asthma, thrombosis or cachexia.

The term “to control weight” encompasses the loss of body mass or the reduction of weight gain over time.

In the present description and in the claims, “appetency disorders” or “appetite disorders” are understood as meaning disorders associated with a substance and especially abuse of a substance and/or dependency on a substance, disorders of food behaviors, especially those liable to cause excess weight, irrespective of its origin, for example: bulimia, appetency for sugars, non-insulin-dependent diabetes. Appetizing substances are therefore understood as meaning substances to be taken into the body and for which an appetite or craving for such consumption by any route of entry. Appetizing substances includes, foods, and their appetizing ingredients such as sugars, carbohydrates, or fats, as well as drinking alcohol or drugs of abuse or excess consumption. An “appetite” may be directed toward such substances as foods, sugars, carbohydrates, fats, as well as ethanol or drugs of abuse or addiction or excess consumption (e.g., tobacco, CNS depressants, CNS stimulants).

An activation assay is an assay that provides an assessment of the in vivo activation of transcription activators in response to extracellular stimuli. The assessment may be provided by measurement of reporter gene activation, measurement of PPARα mRNA levels, or proliferation of cells transfected with PPARα. It includes assays wherein the activation of PPARα that results from PPARα —RXR heterodimer formation that results from binding of a PPARα subtype specific ligand to PPARα.

An agonist is a ligand of a receptor which activates the receptor or causes signal transduction upon binding to the receptor. OEA is an example of a PPARα receptor agonist.

An antagonist is a ligand of a receptor which binds to the receptor but does not appreciably activate the receptor or appreciably cause signal transduction. An antagonist may block the ability of an agonist to bind and activate a receptor or otherwise reduce the activity of the receptor under physiological conditions.

A binding assay is an assay that provides an assessment of ligand binding to a receptor (e.g., PPARα, PPARβ, or PPARγ receptors). For instance, the assessment may be provided by measurement of displacement of radioactively labeled PPARα ligand, of electrophoretic mobility shifts, measurement of immunoprecipitation of PPARα, PPARβ, or PPARγ to antibodies. The assessment may be accomplished through high throughput screening. A “specific” binder or binding of PPARα refers to a compound or binding interaction that has at least 5 fold greater affinity (e.g., as measured by EC₅₀'s or IC₅₀'s) for PPARα than for PPARγ or for PPARβ. Binding is not determinative that a ligand is an agonist or an antagonist.

A peroxisome proliferator activated receptor (PPAR) is a member of a family of nuclear receptors, distinguished in α, β, and γ subtypes as described herein.

A specific or selective PPAR activator is a compound that preferentially binds and activates one PPAR subtype over another. For example, a specific activator of PPARα is OEA.

A specific or selective binder is a compound that preferentially binds one PPAR subtype over another. For example, a specific binder of PPARα is OEA.

Compounds of the Invention

Compounds of the present invention (OEA-like compounds, OEA-like modulators, FAAH inhibitors) may possess asymmetric carbon atoms (optical centers) or double bonds; the racemates, diastereomers, geometric isomers and individual isomers are all intended to be encompassed within the scope of the present invention.

Such compounds of the invention may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of such a compound of the invention may be obtained by stereospecific synthesis using optically pure starting materials of known configuration.

The compounds of the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention.

The instant compounds may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counterion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

The invention also encompasses prodrugs of OEA-like compounds, OEA-like modulators, and FAAH inhibitors which on administration undergo chemical conversion by metabolic processes before becoming active pharmacological substances. In general, such prodrugs will be derivatives of the present compounds that are readily convertible in vivo into a functional compound of the invention. Conventional procedures for the selection and preparation of suitable prodrug derivatives are described, for example, in “Design of Prodrugs”, ed. H. Bundgaard, Elsevier, 1985. The invention also encompasses active metabolites of the present compounds.

A. Fatty Acid Alkanolamide Compounds, Homologs, and Analogs.

OEA-like compounds and OEA-like modulators of the invention include, but are not limited to fatty acid ethanolamide compounds, and their homologues. A variety of OEA-like compounds ane OEA-like modulators are contemplated. These compounds include compounds having the following general formula:

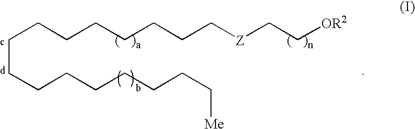

In this formula, n is any number from 0 to 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of unsubstituted or unsubstituted alkyl, hydrogen, substituted or unsubstituted C₁-C₆ alkyl, substituted or unsubstituted lower (C₁-C₆) acyl, homoalkyl, and aryl. Up to eight hydrogen atoms of the compound may also be substituted by methyl group or a double bond. In addition, the molecular bond between carbons c and d may be unsaturated or saturated. In some embodiments, the fatty acid ethanolamide of the above formula is a naturally occurring compound. In some preferred embodiments, the alkyl subsitutents are each homoalkyl.

OEA-like compounds and OEA-like modulators of the invention also include compounds of the following formula:

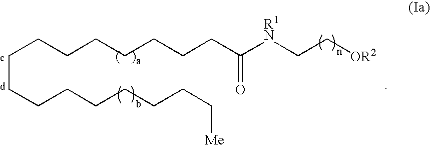

In one embodiment, the compounds of Formula Ia have n from 0 to 5; and a sum of a and b that is from 0 to 4; and members R¹ and R² independently selected from the group consisting of hydrogen, substituted or unsubstituted C₁-C₆ alkyl, lower substituted or unsubstituted (C₁-C₆) acyl, homoalkyl, and substituted or unsubstituted aryl. In this embodiment, up to eight hydrogen atoms of the fatty acid portion and alkanolamine (e.g., ethanolamine) portion of compounds of the above formula may also be substituted by methyl or a double bond. In addition, the molecular bond between carbons c and d may be unsaturated or saturated. In some embodiments with acyl groups, the acyl groups may be the propionic, acetic, or butyric acids and attached via an ester linkage as R² or an amide linkage as R¹. In some embodiments, a H atom attached to a carbon atom of a compound of the above formula is replaced with a halogen atom, preferably a Cl atom or a F atom.

In another embodiment, the above compounds particularly include those in which the fatty acid moiety comprises oleic acid, elaidic acid, or palmitic acid. Such compounds include oleoylethanolamide, elaidylethanolamide and palmitoylethanolamide.

In still another embodiment, the compounds of Formula Ia have n from 1 to 3; and a sum of a and b that is from 1 to 3; and members R¹ and R² independently selected from the group consisting of hydrogen, substituted or unsubstituted C₁-C₆ alkyl, and lower substituted or unsubstituted (C₁-C₆) acyl. In this embodiment, up to four hydrogen atoms of the fatty acid portion and alkanolamine (e.g., ethanolamine) portion of compounds of the above formula may also be substituted by methyl or a double bond. In addition, the molecular bond between carbons c and d may be unsaturated or saturated. In a further embodiment, the molecular bond between carbons c and d is unsaturated and no other hydrogen atoms are substituted. In a still further embodiment thereof, the members R¹ and R² are independently selected from the group consisting of hydrogen, substituted or unsubstituted C₁-C₃ alkyl, and substituted or unsubstituted lower (C₁-C₃) acyl.

Exemplary compounds provide mono-methyl substituted compounds, including ethanolamides, of Formula Ia. Such compounds include:

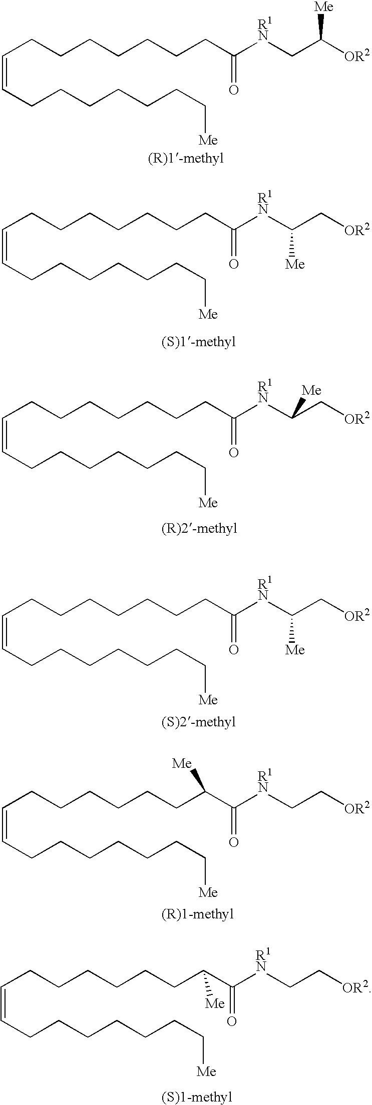

The methyl substituted compounds of the above formula include particularly those compounds where R¹ and R² are both H: (R)1′-methyloleoylethanolamide, S(1′)-methyloleoylethanolamide, (R)2′-methyloleoylethanolamide, (S)2′-methyloleoylethanolamide, (R)1-methyloleoylethanolamide, and (S)1-methyloleoylethanolamide.

Reverse OEA-Like Compounds.

OEA-like compounds and OEA-like modulators of the invention also include a variety of analogs of OEA. These compounds include reverse OEA compounds of the general formula:

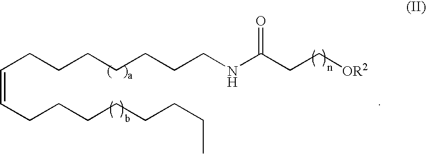

In some embodiments, the invention provides compounds of Formula II. Exemplary the compounds of Formula II have n from 1 to 5, and a sum of a and b from 0 to 4. In this embodiment, the member R² is selected from the group consisting of hydrogen, substituted or unsubstituted C₁-C₆ alkyl, substituted or unsubstituted lower (C₁-C₆) acyl, homoalkyl, and aryl. In addition, up to four hydrogen atoms of either or both the fatty acid portion and alkanolamine (e.g., ethanolamine) portion of compounds of the above formula may also be substituted by methyl or a double bond.

Exemplary compounds of formula II include those compounds where the alkanolamine portion is ethanolamine, compounds where R is H, and compounds where a and b are each 1, and compounds where n is 1.

One embodiment of a compound according to Formula II is

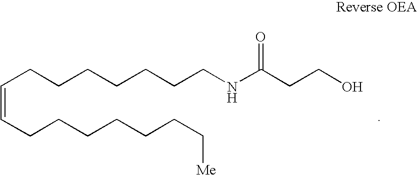

In another embodiment, the compounds of Formula II have n from 1 to 5 and a sum of a and b from 1 to 3. In this embodiment, the member R² is selected from the group consisting of hydrogen, substituted or unsubstituted C₁-C₆ alkyl, and substituted or unsubstituted lower (C₁-C₆) acyl. In addition, up to four hydrogen atoms of either or both the fatty acid portion and alkanolamine (e.g., ethanolamine) portion of compounds of the above formula may also be substituted by methyl or a double bond.

Oleoylalkanol Ester Compounds.

OEA-like compounds and OEA-like modulators of the invention also include oleoylalkanol esters of the general formula:

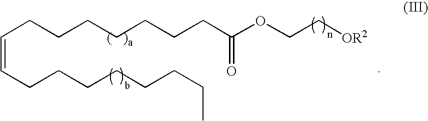

In some embodiments, the compounds of Formula III, have n from 1 to 5; and the sum of a and b from 0 to 4. The member R² is selected from the group consisting of hydrogen, substituted or unsubstituted C₁-C₆ alkyl, lower (C₁-C₆) acyl, homoalkyl, and aryl. Up to four hydrogen atoms of either or both the fatty acid portion and alkanol (e.g., ethanol) portion of compounds of the above formula may also be substituted by methyl or a double bond.

In some embodiments, the compounds of Formula III, have n from 1 to 3; and the sum of a and b from 1 to 3. The member R² is selected from the group consisting of hydrogen, substituted or unsubstituted C₁-C₆ alkyl, and substituted or unsubstituted lower (C₁-C₆) acyl. Up to four hydrogen atoms of the fatty acid portion and alkanol (e.g., ethanol) portion of compounds of the above formula may also be substituted by methyl or a double bond.

Compounds of Formula III include those compounds where R² is H, compounds where a and b are each 1, and compounds where n is 1. Examples of compounds according to Formula III include the oleoyldiethanol ester:

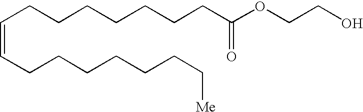

Compounds of Formula III also include mono-methyl substituted oleoyl ethanol esters such as the (R or S)-2′-methyloleoylethanolesters; the (R or S)-1′-methyloleoylethanolesters; and the (R or S))-1′-methyloleoylethanolesters; respectively: 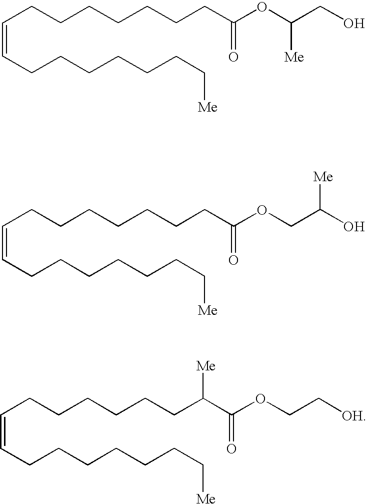
Oleoyl Alkanol Ethers

OEA-like compounds and OEA-like modulators of the invention also include oleoylalkanol ethers according to the general formula:

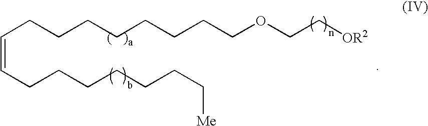

In some embodiments, the compounds of Formula IV, have an n from 1 to 5 and a sum of a and b that can be from 0 to 4. The member R² is selected from the group consisting of hydrogen, substituted or unsubstituted C₁-C₆ alkyl, substituted or unsubstituted lower (C₁-C₆) acyl, alkyl, and substituted and unsubstituted aryl. Up to four hydrogen atoms of either or both the fatty acid portion and alkanol (e.g., ethanol) portion of compounds of the above formula may also be substituted by methyl or a double bond.

In other embodiments, the compounds of Formula IV, have n from 1 to 3; and the sum of a and b can be from 1 to 3. The member R² is selected from the group consisting of hydrogen, substituted or unsubstituted C₁-C₆ alkyl, and substituted or unsubstituted lower (C₁-C₆) acyl. Up to four hydrogen atoms of either or both the fatty acid portion and alkanol (e.g., ethanol) portion of compounds of the above formula may also be substituted by methyl or a double bond.

Compounds of Formula IV include those compounds where R² is H, compounds where a and b are each 1, and compounds where n is 1. Examples of compounds according to Formula IV include the following (R or S) 1′-oleoylethanol ethers and (R or S)-2′-oleoylethanol ethers: 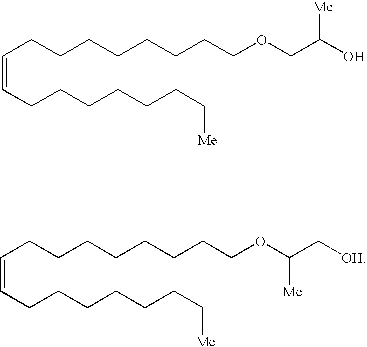
Fatty Acid Alkanolamide Analogs Having Polar Head Variants.

OEA-like compounds and OEA-like modulators of the invention include compounds having a variety of polar head analogs of OEA. These compounds include compounds having a fatty acid moiety of the general formula:

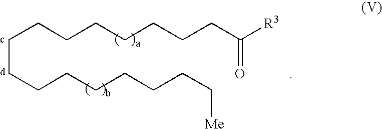

In some embodiments, the compounds of Formula V have a sum of a and b that can be from 0 to 4. In other embodiments, the sum of a and b is from 1 to 3. In these embodiments, up to four hydrogen atoms of the compounds of the above formula may also be substituted by methyl or a double bond. In addition, the molecular bond between carbons c and d may be unsaturated or saturated. A particularly preferred embodiment is that of the oleic acid fatty acid moiety:

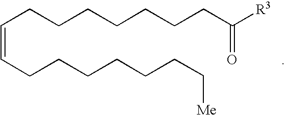

The R³ group of the above structures may be selected from any of the following:

HO—(CH₂)_{z}—NH— wherein z is from 1 to 5, and the alkyl portion thereof is an unbranched methylene chain. For example:

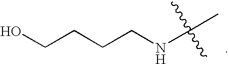

H₂N—(CH₂)_{z}—NH— wherein z is from 1 to 5, and the alkyl portion thereof is an unbranched methylene chain. For example:

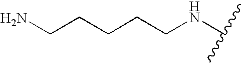

HO—(CH₂)ₓ—NH— wherein x is from 1 to 8, and the alkyl portion thereof may be branched or cyclic. For example,

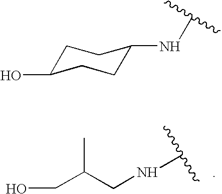

Additional polar head groups for R³ include, for instance, compounds having furan, dihydrofuran and tetrahydrofuran functional groups: 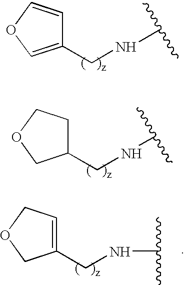
In the above structures, z can be from 1 to 5.

Such compounds of the invention include, for instance, those having R³ polar head groups based upon pyrole, pyrrolidine, and pyrroline rings: 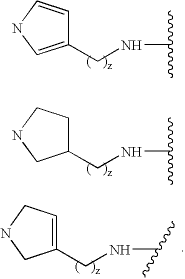
In the compounds of the above structures, z can be from 1 to 5.

Other exemplary polar head groups include a variety of imidazole and oxazoles, for example: 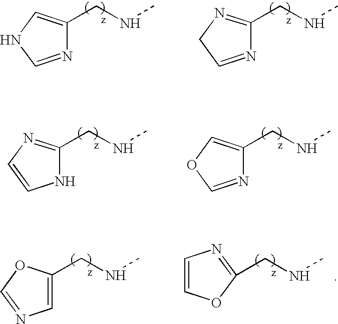
In the compounds of the above structures, z can be from 1 to 5.

Oxazolpyridine polar head groups are also exemplary: 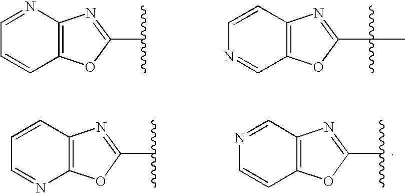
Fatty Acid Alkanolamide Analogs Having Apolar Tail Variants.

OEA-like compound and OEA-like modulators of the invention include a variety of alkanolamide and ethanolamide compounds having a variety of flexible apolar tails. These compounds include compounds of the following formulas in which R represents an ethanolamine moiety, an alkanolamine moiety, or a stable analog thereof. In the case of ethanolamine, the ethanolamine moiety is attached preferably via the ethanolamine nitrogen rather than the ethanolamine oxygen. 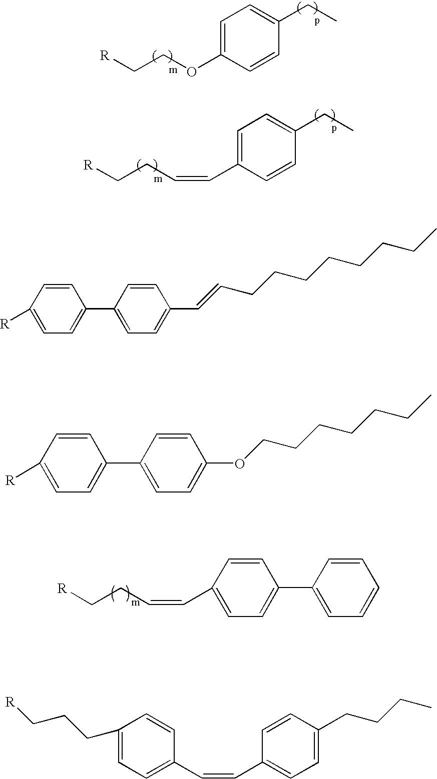
In the above structures, m is from 1 to 9 and p is independently from 1 to 5.

An exemplary compound is:

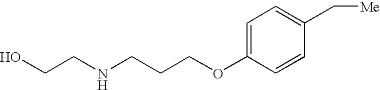

Another exemplary compound is an ethanolamine analog with an apolar tail of the following structural formula:

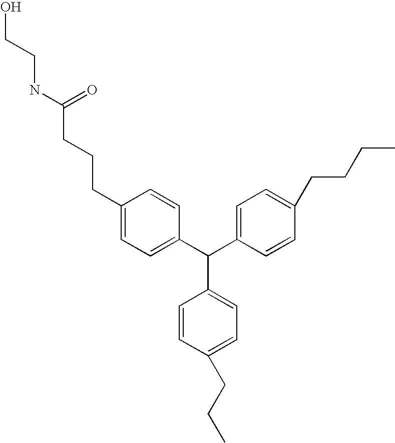

OEA-like compound and OEA-like modulators of the invention of the invention include those disclosed in U.S. patent application Ser. No. 10/112,509 filed March, 27, 2002, assigned to the same assignee as the present application, which is incorporated herein by reference. In other embodiments, the fatty acid moiety of the fatty acid alkanolamide or ethanolamide compound, homologue, or analog may be saturated or unsaturated, and if unsaturated may be monounsaturated or polyunsaturated.

In some embodiments, the fatty acid moiety of the fatty acid alkanolamide compound, homologue, or analog is a fatty acid selected from the group consisting of oleic acid, palmitic acid, elaidic acid, palmitoleic acid, linoleic acid, α-linolenic acid, and γ-linolenic acid. In certain embodiments, the fatty acid moieties have from twelve to 20 carbon atoms.

Other embodiments are provided by varying the hydroxyalkylamide moiety of the fatty acid amide compound, homologue or analog. These embodiments include the introduction of a substituted or unsubstituted lower (C₁-C₃) alkyl group on the hydroxyl group of an alkanolamide or ethanolamide moiety so as to form the corresponding lower alkyl ether. In another embodiment, the hydroxy group of the alkanolamide or ethanolamide moiety is bound to a carboxylate group of a C₂ to C₆ substituted or unsubstituted alkyl carboxylic acid to form the corresponding ester of the fatty acid ethanolamide. Such embodiments include fatty acid alkanolamide and fatty acid ethanolamides in ester linkage to organic carboxylic acids such as acetic acid, propionic acid, and butanoic acid. In one embodiment, the fatty acid alkanolamide is oleoylalkanolamide. In a further embodiment, the fatty acid alkanolamide is oleoylethanolamide.

In still another embodiment, the fatty acid ethanolamide compound, homologue, or analog further comprises a substituted or unsubstituted lower alkyl (C₁-C₃) group covalently bound to the nitrogen atom of the fatty acid ethanolamide.

In still another embodiment, the compound of the invention is fatty acid alkanolamide compound or homologue satisfying the following formula VI:

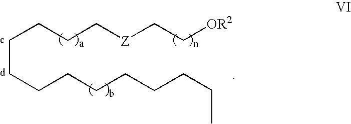

In this formula, n is any number from 0 to 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of unsubstituted or unsubstituted alkyl, hydrogen, substituted or unsubstituted C₁-C₆ alkyl, substituted or unsubstituted lower (C₁-C₆) acyl, homoalkyl, and aryl. Up to six hydrogen atoms of the compound may also be substituted by methyl group or a double bond. In addition, the molecular bond between carbons c and d may be unsaturated or saturated. In some embodiments, the fatty acid ethanolamide of the above formula is a naturally occurring compound. In some preferred embodiments, the alkyl subsitutents are each homoalkyl, or its pharmaceutically acceptable salt. Further embodiments of the compounds of Formula VI have substituents as set forth for compounds of Formula I above. In some embodiments, a H atom attached to a carbon atom of a compound of the above formula is replaced with a halogen atom, preferably a Cl atom or a F atom.

Synthesis of Fatty Acid Alkanolamides

Compounds useful in practicing the present invention can be readily synthesized and purified using methods recognized in the art. In an exemplary synthetic scheme (Scheme 1), a carboxylic acid and an aminoalcohol (or an O-protected derivative thereof) are reacted in a the presence of a dehydrating agent, e.g., dicyclohexylcarbodiimide, in an appropriate solvent. The fatty acid alkanol amide is isolated by methods such as extraction, crystallization, precipitation, chromatography and the like. If the final product is the O-protected adduct, it is deprotected, typically by an art-recognized method, to afford a fatty acid adduct having a free hydroxyl group.

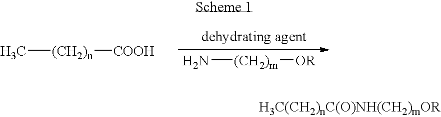

Those of skill in the art will recognize that many variants on the scheme set forth above are available. For example, an activated derivative, e.g, acyl halide, active ester, of the acid can be used. Similarly, a glycol (preferably mono O-protected) can be substituted for the amino alcohol, resulting in an ester linkage between the two constituents of the molecule.

Reverse esters and reverse amides can also be readily synthesized by art-recognized methods. For example, a hydroxycarboxylic acid is reacted with an amine or hydroxy derivative of a long chain alkyl (i.e., C₄-C₂₂) in the presence of a dehydrating agent. In certain reaction pathways, it is desirable to protect the hydroxyl moiety of the hydroxycarboxylic acid.

Ethers and mercaptans can be prepared by methods well-known to those of skill in the art, e.g., Williamson synthesis. For example, a long chain alkyl alcohol or thiol is deprotonated by a base, e.g, NaH, and a reactive alcohol derivative, e.g., a halo, tosyl, mesyl alcohol, or a protected derivative thereof is reacted with the resulting anion to form the ester or mercaptan.

The above-recited methods and variations thereof can be found in, for example, RECENT DEVELOPMENTS IN THE Synthesis OF FATTY ACID DERIVATIVES, Knothe G, ed., Amer. Oil Chemists Society 1999; COMPREHENSIVE NATURAL PRODUCTS CHEMISTRY AND OTHER SECONDARY METABOLITES INCLUDING FATTY ACIDS AND THEIR DERIVATIVES, Nakanishi K, ed., Pergamon Press, 1999; ORGANIC SYNTHESIS COLLECTED VOLUMES I-V, John Wiley and Sons; COMPENDIUM OF ORGANIC SYNTHETIC METHODS, Volumes 1-6, Wiley Interscience 1984; ORGANIC FUNCTIONAL GROUP PREPARATION, Volumes I-III, Academic Press Ltd. 1983; Greene T, PROTECTING GROUPS IN ORGANIC SYNTHESIS, 2d ed., Wiley Interscience 1991.

OEA-Like Modulators which are Not OEA-Like Compounds

In addition, OEA-like modulators need not be an OEA-like compound (e.g., OEA, fatty acid amide or homolog thereof). In some embodiments, the OEA-like modulator is a compound such as taught in U.S. Pat. No. 6,200,998 (hereby incorporated by reference) that are PPARα activators. This reference teaches PPAR agonist compounds of the general formula:

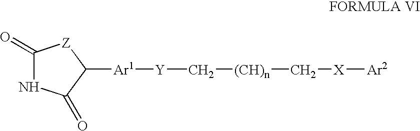

In the above formula, Ar¹ is (1) arylene or (2) heteroarylene, wherein arylene and heteroarylene are optionally substituted with from 1 to 4 groups selected from R^{a} (defined below); Ar² is (1) ortho-substituted aryl or (2) ortho-substituted heteroaryl, wherein said ortho substituent is selected from R (defined below); and aryl and heteroaryl are optionally further substituted with from 1-4 groups independently selected from R^{a}; X and Y are independently O, S, N-Rb (defined below), or CH₂; Z is O or S; n is 0 to 3; R is (1) C₃₋₁₀ alkyl optionally substituted with 1-4 groups selected from halo and C₃₋₆ cycloalkyl, (2) C₃₋₁₀ alkenyl, or (3) C₃₋₈ cycloalkyl; R^{a} is (1) C₁₋₁₅ alkanoyl, (2) C₁₋₁₅ alkyl, (3) C₂₋₁₅ alkenyl, (4) C₂₋₁₅ alkynyl, (5) halo, (6) OR^{b}, (7) aryl, or (8) heteroaryl, wherein said alkyl, alkenyl, alkynyl, and alkanoyl are optionally substituted with from 1-5 groups selected from R^{c} (defined below), and said aryl and heteroaryl optionally substituted with 1 to 5 groups selected from R^{d} (defined below); Rb is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl, wherein alkyl, alkenyl, alkynyl are optionally substituted with one to four substituents independently selected from R^{c}, and cycloalkyl, aryl and heteroaryl are optionally substituted with one to four substituents independently selected from R^{d}; or R^{c} is (1) halo, (2) aryl, (3) heteroaryl, (4) CN, (5) NO₂, (6) OR^{f}; (7) S(O)ₘR^{f}, m=0, 1 or 2, provided that R^{f} (defined below) is not H when m is 1 or 2; (8) NR^{f}R^{f} (9) NR^{f}COR^{f}, (10) NR^{f}CO₂ R^{f}, (11) NR^{f}CON(R^{f})₂, (12) NR^{f} SO₂ R^{f}, provided that R^{f} is not H, (13) COR^{f}, (14) CO₂R^{f}, (15) CON(R^{f})₂, (16) SO₂ N(R^{f})₂, (17) OCON(R^{f})₂, or (18) C₃₋₈ cycloalkyl, wherein said cycloalkyl, aryl and heteroaryl are optionally substituted with 1 to 3 groups of halo or C₁₋₆ alkyl; R^{d} is (1) a group selected from R^{c}, (2) C₁₋₁₀ alkyl, (3) C₂₋₁₀ alkenyl, (4) C₂₋₁₀ alkynyl, (5) aryl C₁₋₁₀ alkyl, or (6) heteroaryl C₁₋₁₀ alkyl, wherein alkyl, alkenyl, alkynyl, aryl, heteroaryl are optionally substituted with a group independently selected from R^{e}; R^{e} is (1) halogen, (2) amino, (3) carboxy, (4) C₁₋₄ alkyl, (5) C₁₋₄ alkoxy, (6) hydroxy, (7) aryl, (8) aryl C₁₋₄ alkyl, or (9) aryloxy; R^{f} is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl; wherein alkyl, alkenyl, alkynyl, aryl, heteroaryl, alkanoyl and cycloalkyl are optionally substituted with one to four groups selected from R^{e}.

Also preferred are those PPARα specific activators as taught in U.S. Pat. No. 5,859,051. These activators have the following general formula as set forth in the U.S. Pat. No. 5,589,051:

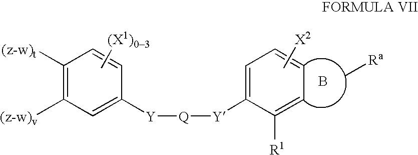

In the embodiments according to Formula VII, R¹ is selected from a group consisting of: H, C₁₋₁₅ alkyl, C₂₋₁₅ alkenyl, C₂₋₁₅ alkynyl and C₃₋₁₀ cycloalkyl, said alkyl, alkenyl, alkynyl, and cycloalkyl optionally substituted with 1 to 3 groups of R^{a} (defined below); R³ is selected from a group consisting of: H, NHR′, NHacyl, C₁₋₁₅ alkyl, C₃₋₁₀ cycloalkyl, C₂₋₁₅ alkenyl, C₁₋₁₅ alkoxy, CO₂ alkyl, OH, C₂₋₁₅ alkynyl, C₅₋₁₀ aryl, C₅₋₁₀ heteroaryl said alkyl, cycloalkyl, alkenyl, alkynyl, aryl and heteroaryl optionally substituted with 1 to 3 groups of R^{a}; (Z—W—) is Z-CR⁶R⁷—, Z—CH.═CH—, or:

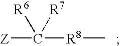

R⁸ is selected from the group consisting of CR⁶R⁷, O, NR⁶, and S(O)ₚ; R⁶ and R⁷ are independently selected from the group consisting of H, C₁₋₆ alkyl; B is selected from the group consisting of: 1) a 5 or 6 membered heterocycle containing 0 to 2 double bonds, and 1 heteroatom selected from the group consisting of O, S and N, the heteroatom being substituted at any position on the five or six membered heterocycle, the heterocycle being optionally unsubstituted or substituted with 1 to 3 groups of R^{a}; 2) a 5 or 6 membered carbocycle containing 0 to 2 double bonds, the carbocycle optionally unsubstituted or substituted with 1 to 3 groups of R^{a} at any position on the five or six membered carbocycle; and 3) a 5 or 6 membered heterocycle containing 0 to 2 double bonds, and 3 heteroatoms selected from the group consisting of O, N, and S, which are substituted at any position on the five or six membered heterocycle, the heterocycle being optionally unsubstituted or substituted with 1 to 3 groups of R^{a}; X¹ and X² are independently selected from a group consisting of: H, OH, C₁₋₁₅ alkyl, C₂₋₁₅ alkenyl, C₂₋₁₅ alkynyl, halo, OR³, ORCF₃, C₅₋₁₀ aryl, C₅₋₁₀ aralkyl, C₅₋₁₀ heteroaryl and C₁₋₁₀ acyl, said alkyl, alkenyl, alkynyl, aryl and heteroaryl optionally substituted with 1 to 3 groups of R^{a}; R^{a} represents a member selected from the group consisting of: halo, acyl, aryl, heteroaryl, CF₃, OCF₃, —O—, CN, NO₂, R³, OR³; SR³, ═N(OR), S(O)R³, SO₂R³, NR³R³, NR³COR³, NR³ CO₂ R³, NR³CON(R³)₂, NR³ SO₂ R³, COR³, CO₂R³, CON(R³)₂, SO₂ N(R³)₂, OCON(R³)₂ said aryl and heteroaryl optionally substituted with 1 to 3 groups of halo or C₁₋₆ alkyl; Y is selected from the group consisting of: S(O)ₚ, —CH₂—, —C(O)—, —C(O)NH—, —NR—, —O—, —SO₂NH—, —NHSO₂; Y¹ is selected from the group consisting of: O and C; Z is selected from the group consisting of: CO₂R³, R³CO₂R³, CONHSO₂Me, CONHSO₂, CONH₂ and 5-(1H-tetrazole); t and v are independently 0 or 1 such that t+v=1 Q is a saturated or unsaturated straight chain hydrocarbon containing 2-4 carbon atoms and p is 0-2 with the proviso when Z is CO₂ R³ and B is a 5 membered heterocycle consisting of O, R³ does not represent methyl.

Additional compounds suitable for practicing the inventive methods include compounds taught in U.S. Pat. No. 5,847,008, U.S. Pat. No. 6,090,836 and U.S. Pat. No. 6,090,839, U.S. Pat. No. 6,160,000 each of which is herein incorporated by reference in its entirety to the extent not inconsistent with the present disclosure.

Additionally a variety of suitable PPAR agonists and activators for screening are taught in U.S. Pat. No. 6,274,608. Aryl and heteroaryl acetic acid and oxyacetic acid compounds are taught for instance in U.S. Pat. No. 6,160,000; substituted 5-aryl-2,4-thiazolidinediones are taught in U.S. Pat. No. 6,200,998; other compounds including PPARa-specific polyunsaturated fatty acids and eicosanoids are known as described in Forman, B M, Chen, J, and Evans R M, PNAS 94:4312-4317 and PCT Patent Publication No. WO 97/36579, published Oct. 9, 1997). The compositions of these publications, which are each herein incorporated by reference in their entirety to the extent not inconsistent with the present disclosure can be screened by the methods provide below to provide the PPARα specific agonists of the invention which are useful, for instance, in reducing body fat. and body weight, modulating fat catabolism, and reducing appetite according to the present disclosure.

Each of the above patents cited in this section are incorporated by reference herein with particular reference to the compounds and compositions they disclose.

Pharmaceutical Compositions.

Another aspect of the present invention provides pharmaceutical compositions which comprise at least one of an agent selected from the group consisting of a FAAH inhibitor, an OEA-like compound and an OEA-like modulator and a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend in part on the nature and severity of the conditions being treated and on the nature of the active ingredient. An exemplary route of administration is the oral route. The compositions may be conveniently presented in unit dosage form and prepared by any of the methods well-known in the art of pharmacy.

In practical use, the FAAH inhibitors, OEA-like compounds, and OEA-like modulators of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers can be employed. If desired, tablets may be coated by standard aqueous or nonaqueous techniques. Such compositions and preparations can contain at least 0.1 percent of active compound. The percentage of active compound in these compositions may, of course, be varied and may conveniently be between about 2 percent to about 60 percent of the weight of the unit. The amount of active compound in such therapeutically useful compositions is such that a therapeutically effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. To prevent breakdown during transit through the upper portion of the GI tract, the composition may be an enteric coated formulation.

Administration

The pharmaceutical compositions of the invention may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The FAAH inhibitors, OEA-like compounds, and OEA-like modulators can each be effective over a wide dosage range. For example, in the treatment of adult humans, dosages from about 10 to about 1000 mg, about 100 to about 500 mg or about 1 to about 100 mg may be needed. Doses of the 0.05 to about 100 mg, and more preferably from about 0.1 to about 100 mg, per day may be used. A most preferable dosage is about 0.1 mg to about 70 mg per day. In choosing a regimen for patients, it may frequently be necessary to begin with a dosage of from about 2 to about 70 mg per day and when the condition is under control to reduce the dosage as low as from about 0.1 to about 10 mg per day. For example, in the treatment of adult humans, dosages from about 0.05 to about 100 mg, preferably from about 0.1 to about 100 mg, per day may be used. The exact dosage will depend upon the mode of administration, on the therapy desired, form in which administered, the subject to be treated and the body weight of the subject to be treated, and the preference and experience of the physician or veterinarian in charge.

Generally, the FAAH inhibitors, OEA-like compounds, and OEA-like modulators can be dispensed in unit dosage form comprising preferably from about 0.1 to about 100 mg of active ingredient together with a pharmaceutically acceptable carrier per unit dosage. Usually, dosage forms suitable for oral, nasal, pulmonary or transdermal administration comprise from about 0.001 mg to about 1000 mg, preferably from about 0.1 mg to about 100 mg of the compounds admixed with a pharmaceutically acceptable carrier or diluent. For storage and use, these preparations preferably contain a preservative to prevent the growth of microorganisms.

Administration of an appropriate amount of the compounds may be by any means known in the art such as, for example, oral or rectal, parenteral, intraperitoneal, intravenous, subcutaneous, subdermal, intranasal, or intramuscular. In some embodiments, administration is transdermal. An appropriate amount or dose of the candidate compound may be determined empirically as is known in the art. For example, with respect to body fat or loss of body weight, an appropriate or therapeutic amount is an amount sufficient to effect a loss of body fat or a loss in body weight in the animal over time. The candidate compound can be administered as often as required to effect a loss of body fat or loss in body weight, for example, hourly, every six, eight, twelve, or eighteen hours, daily, or weekly

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Injection solutions and suspensions can be prepared from sterile powders, granules, and tablets of the kind previously described. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

With respect to transdermal routes of administration, methods for transdermal administration of drugs are disclosed in Remington's Pharmaceutical Sciences, 17th Edition, (Gennaro et al. Eds., Mack Publishing Co., 1985). Dermal or skin patches are a preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012, 6,261,595, and 6,261,595. Each of which is incorporated by reference in its entirety.

Preferred patches include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

The FAAH inhibitors, OEA-like compounds and OEA-like modulators of the invention may be used in combination with other compounds of the invention or with other drugs that may also be useful in dieting or the treatment, prevention, suppression or amelioration of body fat. Such other drugs may be administered, by a route and in an amount commonly used therefore, contemporaneously or sequentially with a compound of the invention. When a FAAH inhibitor or OEA-like compound or OEA-like modulator of the invention is used contemporaneously with one or more other drugs, a pharmaceutical composition in unit dosage form containing such other drugs and the compound is preferred. When used in combination with one or more other active ingredients, the compound of the present invention and the other active ingredients may be used in lower doses than when each is used singly. Accordingly, the pharmaceutical compositions of the present invention include those that contain one or more other active ingredients, in addition to the compounds disclosed above.

The pharmaceutically or physiologically acceptable salts include, but not limited to, a metal salts such as sodium salt, potassium salt, lithium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

Methods of Treatment

In one aspect, the invention provides a method of treating, controlling or preventing one or more diseases, disorders, or conditions in a subject mediated by the PPARα receptor or responsive to administration of a PPARα modulator by administering one or more of an agent selected from the group consisting of a FAAH inhibitor, an OEA-like compound and an OEA-like modulator. Such conditions include, but are not limited to, diabetes mellitus, hyperglycemia, obesity, hyperlipidemia, hypertriglyceridemia, hypercholesterolemia, atherosclerosis, vascular restenosis, irritable bowel syndrome, pancreatitis, abdominal obesity, adipose cell tumors, adipose cell carcinomas, Syndrome X, polycystic ovarian syndrome, and other disorders where insulin resistance is a component; metabolic disorders, excess body fat, cellulite, Type II diabetes, insulin resistance, artherogenesis, an inflammatory disorder or condition, Alzheimers disease, Crohn's disease, a vascular inflammation, an inflammatory bowel disorder, rheumatoid arthritis, asthma, thrombosis and cachexia. In some embodiments, the subject is human. In some embodiments, the modulator is a fatty acid alkanolamide. In some embodiments, the compound is a modulator of Formula I or Formula VI.

The FAAH inhibitors, OEA-like compounds (e.g., fatty acid alkanolamides, fatty acid ethanolamide compounds, analogs, and homologues with PPARα modulatory activity), and/or OEA-like modulators, their compositions and methods of administration can be used to reduce body fat and or body weight in mammals, including dogs, cats, and especially humans. The weight loss may be for aesthetic or therapeutic purposes. The compounds may also be used to reduce appetite or induce hypophagia.

The FAAH inhibitors, OEA-like compounds and/or OEA-like modulators, and their compositions can be administered to subjects (e.g., humans) to prevent weight gain or body fat increases in individuals within a normal weight range. The compounds may be used in otherwise healthy individuals who are not otherwise in need of any pharmaceutical intervention for diseases related to diabetes or hyperlipidemia or cancer. In some embodiments, the individuals to be treated are free of diseases related to disturbances in sugar or lipid levels or metabolism or free of risk factors for cardiovascular and cerebrovascular disease. The individuals may be non-diabetic and have blood sugar levels in the normal range. The individuals may also have blood lipids (e.g., cholesterol) or triglyceride levels in the normal range. The individuals may be free of atherosclerosis. The individuals may be free of other conditions such as cancer or other tumors, disorders involving insulin resistance, Syndrome X, and pancreatitis.

In other embodiments, the subjects are overweight or obese persons in need of body fat and/or body weight reduction. In these embodiments, the methods, compounds, and compositions of the invention can be administered to promote weight loss and also to prevent weight gain once a body weight within the normal range for a person of that sex and age and height has been achieved. The FAAH inhibitors, OEA-like compounds and/or OEA-like modulators may be used in otherwise healthy individuals who are not in need of any pharmaceutical treatment of a disorder related to diabetes, hyperlipidemia, or cancer. The individuals may also otherwise free of risk factors for cardiovascular and cerebrovascular diseases. In some embodiments, the individuals to be treated are free of diseases related to sugar (e.g., glucose) or lipid metabolism. The individuals may be non-diabetic and have blood sugar levels in the normal range. The individuals may also have blood lipids (e.g., cholesterol, HDL, LDL, total cholesterol) or triglyceride levels in the normal range. The individuals may not need to be in treatment for atherosclerosis.

The FAAH inhibitors, OEA-like compounds and/or OEA-like modulators compositions of the invention may also be administered to suppress appetite in mammals, including cats, dogs, and humans. In some embodiments, the compounds may be used in otherwise healthy individuals who are not in need of pharmaceutical interventions for any disease. In some embodiments, the individuals do not need preventive or ameliorative therapy for diseases, including cancer, diabetes, or hyperlipidemia. In some embodiments, the individuals to be treated are free of diseases related to abnormal sugar or lipid levels. In other embodiments the individuals may be free of risk factors for cardiovascular or cerebrovascular disease. The individuals may be non-diabetic and have blood sugar levels in the normal range. The individuals may also have blood lipids (e.g., cholesterol) or triglyceride levels in the normal range. The individuals may be free of atherosclerosis.

In some embodiments, the methods and compositions of the present invention act may act selectively, for instance, on consumption behavior disorders pertaining to appetizing substances. Thus, administration of the inventive compositions and such compounds can make it possible to regulate the desire to consume non-essential items such as excess sugars, excess carbohydrates, fats, alcohol or drugs.

The FAAH inhibitors, OEA-like compounds and/or OEA-like modulators, methods, and compositions of the invention may also be administered to modulate fat metabolism (e.g., increase fat catabolism) in mammals, including cats, dogs, and humans. In some embodiments, the compounds may be used to reduce appetite in otherwise healthy individuals. In some embodiments, the individuals to be treated are free of diseases related to sugar or lipid metabolism (e.g., diabetes, hypercholesterolemia, low HDL levels or high LDL levels). The individuals may be non-diabetic and have blood sugar levels in the normal range. The individuals may also have blood lipids (e.g., cholesterol) or triglyceride levels in the normal range. The individuals may be free of atherosclerosis.

Treatment with the FAAH inhibitors, OEA-like compounds and/or OEA-like modulators of the invention may be prophylactic or to prevent progression of harm preventable by activation of PPARα receptors. The duration and frequency of treatment can be according to the severity of the disease or condition, its chronicity, and responsiveness to treatment. A treatment may be short-term over days or weeks or chronic for months to years. One of ordinary skill in the art will be able to determine when a subject is responding favorably to an administered agent (e.g., by measuring blood lipids, weight, blood sugar or insulin levels, inflammatory cytokines, or other objective and subjective signs or symptoms of the subject diseases and conditions). In some embodiments, treatment with the compounds and compositions of the invention may be reduced or terminated once a predetermined parameter as been reached has been accomplished. For instance, with respect to weight loss as an objective, the administration can be terminated when the desired amount amount of weight loss has been accomplished or when the individual achieves a BMI within the normal range.

The FAAH inhibitors, OEA-like compounds and/or OEA like modulators may be administered solely for the purposes of reducing body fat or reducing appetite. These compounds may be administered topically and locally in the treatment of cellulite. Such compounds may be administered in the form of a topical spary, cream, powder or ointment or a dermal patch.

In some embodiments, the FAAH inhibitor, OEA-like compound and/or OEA-like modulator is administered with a second agent, including but not limited to, an agent selected from the group consisting of insulin sensitizers, PPAR.γ. agonists, glitazones, troglitazone, pioglitazone, englitazone, MCC-555, BRL49653, biguanides, metformin, phenformin, insulin, insulin mimetics, sulfonylureas, tolbutamide, glipizide, α-glucosidase inhibitors, acarbose, cholesterol lowering agents, HMG-CoA reductase inhibitors, lovastatin, simvastatin, pravastatin, fluvastatin, atorvastatin, rivastatin, other statins, sequestrants, cholestyramine, colestipol, dialkylaminoalkyl derivatives of a cross-linked dextran, nicotinyl alcohol, nicotinic acid, a nicotinic acid salt, PPARβ agonists, fenofibric acid derivatives, gemfibrozil, clofibrate, fenofibrate, benzafibrate, inhibitors of cholesterol absorption, β-sitosterol, acyl CoA:cholesterol acyltransferase inhibitors, melinamide, probucol, agonists, antiobesity compounds, fenfluramine, dexfenfluramine, phentiramine, sulbitramine, orlistat, neuropeptide Y5 inhibitors, β₃ adrenergic receptor agonists, and ileal bile acid transporter inhibitors.

Administration of an appropriate amount of the FAAH inhibitor, OEA-like compound or OEA-like modulator or pharmaceutical composition(s) thereof may be by any means known in the art such as, for example, oral or rectal, intraparenteral such as, for example, intraperitoneal, intravenous, subcutaneous, subdermal, intranasal, or intramuscular. Preferably administration is intraperitoneal. An appropriate amount of the candidate compound may be determined empirically as is known in the art. For example, with respect to weight loss, as the objective, an appropriate amount is an amount sufficient to effect a loss of body fat or a loss in body weight in the animal over time. The candidate compound can be administered as often as required to effect a loss of body fat or loss in body weight, for example, hourly, every six, eight, twelve, or eighteen hours, daily, or weekly.

Identification of OEA-Like Compounds and OEA Like Modulators

Identification of compounds that specifically bind PPARα can be accomplished by any means known in the art, such as, for example, electrophoretic mobility shift assays and competitive binding assays. Preferably PPARα specific binding compounds have at least 5-10 fold, preferably 10-100 fold, more preferably 100-500 fold, most preferably greater than 1000 fold specificity for PPARα compared to other PPAR subtypes. Mammalian PPAR subtypes (e.g., rat, mouse, hamster, rabbit, primate, guinea pig) are preferably used. More preferably, human PPAR subtypes are used.

Electrophoretic Mobility Shift Assays

Electrophoretic mobility shift assays can be used to determine whether test compounds bind to PPARα and affect its electrophoretic mobility. (Forman, et al. (1997) PNAS 94:4312 and Kliewer, et al. (1994) PNAS 91:7355). Electrophoretic mobility shift assays involve incubating a PPAR-RXR with a test compound in the presence of a labeled nucleotide sequence. Labels are known to those of skill in the art and include, for example, isotopes such as, ³H, ¹⁴C, ³⁵S, and ³²P, and non-radioactive labels such as fluorescent labels or chemiluminescent labels. Fluorescent molecules which can be used to label nucleic acid molecules include, for example, fluorescein isothiocyanate and pentafluorophenyl esters. Fluorescent labels and chemical methods of DNA and RNA fluorescent labeling have been reviewed recently (Proudnikov et al., 1996, Nucleic Acids Res. 24:4535-42).

Chemiluminescent labels and chemiluminescent methods of labeling DNA and RNA have been reviewed recently (Rihn et al., 1995, J. Biochem. Biophys. Methods 30:91-102). Use of non-radioactive labeled probes directly for studying protein-polynucleotide interactions with EMSA has been described. (U.S. Pat. No. 5,900,358). The mixtures can be separated, run on a separate lane of a gel, and autoradiographed. For example, if a test compound does not result in a change in the bands seen in the control lane then the test compound is not a candidate PPARα specific binding compound. On the other hand, if a change in intensity in at least one of the bands is seen, then the compound is a candidate PPARα specific binding compound. (U.S. Pat. No. 6,265,160). The incubation mixture is then electrophoretically separated and the resulting gel exposed to X-ray film. The resulting autoradiograph may have one or more bands representing slowly migrating DNA-protein complexes. This control lane can indicate the mobility of the complex between the DNA probe and PPAR.

Monoclonal antibodies specific for PPAR subtypes can be used to identify PPARα specific binding compounds in modified electrophoretic mobility shift assays. Purified PPARβ, PPARα or PPARγ can be incubated with an appropriate amount of a test compound in the presence of RXR. For these assays, the test compound need not be labeled. PPAR subtype specific monoclonal antibodies can be incubated with the PPAR-RXR-test compound mixture. For instance, test compounds that bind PPAR induce supershifting of the PPAR-RXR complex on a gel (Forman, et al. (1997), PNAS 94:4312) which can be detected by anti-PPAR monoclonal antibodies using a Western blot (immunoblot).

Generation of monoclonal antibodies has been previously described and can be accomplished by any means known in the art. (Buhring et al. in Hybridoma 1991, Vol. 10, No. 1, pp. 77-78). For example, an animal such as a guinea pig or rat, preferably a mouse is immunized with a purified PPAR subtype, the antibody-producing cells, preferably splenic lymphocytes, are collected and fused to a stable, immortalized cell line, preferably a myeloma cell line, to produce hybridoma cells which are then isolated and cloned. (U.S. Pat. No. 6,156,882).

Western blots generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind PPAR subtypes. These antibodies may be directly labeled or alternatively may be subsequently detected using labeled antibodies (e.g., labeled sheep anti-mouse antibodies) that specifically bind to the anti-PPAR antibodies.

The particular label or detectable group used in the assay is not a critical aspect of the invention, as long as it does not significantly interfere with the specific binding of the PPAR subtype specific ligand used in the assay. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), and colorimetric labels such as colloidal gold or colored glass or plastic beads (e.g., polystyrene, polypropylene, latex, etc.).

The molecules can be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Other assay formats include liposome immunoassays (LIA), which use liposomes designed to bind specific molecules (e.g., antibodies) and release encapsulated reagents or markers. The released chemicals can be then detected according to standard techniques (see Monroe et al., Amer. Clin. Prod. Rev. 5:34-41 (1986)).

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 110C to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Competitive Binding Assays

In addition to electrophoretic mobility shift assays, competitive binding assays can be used to identify PPARα specific binding compounds. In competitive assays, the binding of test compounds to PPARα can be determined by measuring the amount of OEA that they displaced (competed away) from PPARα. Purified PPARβ, PPARα, and PPARγ receptors can be incubated with varying amounts of a test compound in the presence of labeled ligands specific for each PPAR subtype. For example, GW 2433 and L-783483 can be used in conjunction with PPARβ; GW 2331 or OEA can be used in conjunction with PPARα; and rosiglitazone, AD-5075, and SB-236636 can be used in conjunction with PPARγ. Specificity of the test compound for each PPAR subtype can be determined by detection of the amount of labeled ligand that remains bound to each PPAR after incubation with the test compound. Labels are discussed above.

High Throughput Screening of Candidate Compounds that Specifically Bind PPARα

In conjunction with the methods described above, identification of OEA-like compounds and OEA-like modulators can be accomplished via high throughput screening. Conventionally, new chemical entities with useful properties can be generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

High throughput screening methods involve providing a library containing a large number of potential PPARα specific binding compounds (candidate compounds). Such “combinatorial chemical libraries” can be then screened in one or more assays, as described herein, to identify those library members (particular chemical species or subclasses) that display a desired characteristic activity. The compounds thus identified can serve as conventional “lead compounds” or can themselves be used as potential or actual therapeutics.

a. Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library can be formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9):1233).

Preparation and screening of combinatorial chemical libraries are well known to those of skill in the art. Such combinatorial chemical libraries include, but are not limited to, benzodiazepines (U.S. Pat. No. 5,288,514), diversomers such as hydantoins, benzodiazepines and dipeptides (Hobbs et al. (1993) PNAS. USA 90: 6909), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658), and small organic molecule libraries (see, e.g., benzodiazepines, Baum (1993) C&EN, January 18, page 33, thiazolidinones and metathiazanones U.S. Pat. No. 5,549,974, pyrrolidines U.S. Pat. Nos. 5,525,735 and 5,519,134, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Biosciences, Columbia, Md., etc.).

b. High Throughput Assays of Chemical Libraries

Many of the in vitro assays for compounds described herein are amenable to high throughput screening. Preferred assays thus detect activation of transcription (i.e., activation of mRNA production) by the test compound(s), activation of protein expression by the test compound(s), or binding to the gene product (e.g., expressed protein) by the test compound(s).

High throughput assays for the presence, absence, or quantification of particular protein products or binding assays are well known to those of skill in the art. Thus, for example, U.S. Pat. No. 5,559,410 discloses high throughput screening methods for proteins, and U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

Measuring Activation of PPARα

The ability of an OEA-like compound or OEA-like modulator to activate PPARα can be measured using any means known in the art. PPARα activators act by inducing PPARα-RXR heterodimer formation. The PPARα-RXR heterodimer then binds to DNA sequences containing AGGTCAnAGGTCA and activates PPAR target genes. Preferably PPARα activators activate PPARα by at least 5-10 fold, more preferably 10-100 fold, more preferably 100-500 fold, more preferably 500-100 fold, most preferably greater than 1000 fold above base level. PPARα can be transfected into cells. The transfected cells can be then exposed to candidate compounds. Any means known in the art can be used to determine whether PPARα is activated by the candidate compound, such as for example, by measuring levels of reporter gene expression and cell proliferation.

Transfection of PPAR into Cells

Any of the well-known procedures for introducing foreign nucleotide sequences into host cells may be used to transfect PPARα into cells such as, for example, calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). Methods of transfection have also been described in U.S. Pat. Nos. 5,616,745, 5,792,6512, 5,965,404, and 6,051,429 and in Current Protocols in Molecular Biology, Ausubel, et al., ed. (2001). It is only necessary that the particular genetic engineering procedure used be capable of successfully introducing at least one gene into the host cell capable of expressing PPARα. After the expression vector is introduced into the cells, the transfected cells can be cultured under conditions favoring expression of PPARα.

Detection of Reporter Gene Expression

Expression of reporter genes in response to compounds identified as binders of PPARα may also be used to measure PPARα activation. PPARα may be co-transfected with reporter genes known in the art such as, for example, luciferase, β-galactosidase, alkaline phosphatase, fluorescent green protein, or chloramphenicol acetyltransferase. The transfected cells can be exposed to appropriate concentrations of candidate compounds with OEA as a positive control. Reporter gene expression will be induced by compounds that bind and activate PPARα. Thus, compounds that induce reporter gene expression can be identified as activators of PPARα. (Forman, et al. (1997) PNAS 94:4312). Preferably the compounds induce reporter gene expression at levels at least 5-10 fold, more preferably 10-100 fold, more preferably 100-500 fold, more preferably 500-1000 fold, most preferably greater than 1000 fold greater than the negative control.

Proliferation of PPARα Transfected Cells

PPARα activation may also be measured by proliferation of cells transfected with PPARα Cell proliferation can be induced by compounds that bind and activate PPARα, such as, for example, OEA. Thus, PPARα transfected cells can be exposed to appropriate concentrations of candidate compounds with OEA as a positive control. Compounds that induce cells to proliferate can thereby be identified as activators of PPARα. Cell proliferation can be measured, for example, by incorporation of 5′-bromo-2′deoxyuridine or 3H-thymidine as described in Jehl-Pietri, et al., (2000) Biochem J. 350:93 and Zoschke and Messner (1984) Clin. Immunol. Immunopath. 32:29, respectively. Preferably the compounds induce cell proliferation at levels at least 5-10 fold, more preferably 10-100 fold, more preferably 100-500 fold, more preferably 500-1000 fold, most preferably greater than 1000 fold greater than the negative control.

Determining Whether OEA-Like Compounds or OEA-Like Modulators Modulate Fatty Acid Metabolism

Once candidate compounds have been identified, they can be administered to an animal to determine whether the identified agonists are effective as body fat reducing compounds or as modulators of fatty acid metabolism.

Animals can be, for example, obese or normal guinea pigs, rats, mice, or rabbits. Suitable rats include, for example, Zucker rats. Suitable mice include, for example, normal mice, ALS/LtJ, C3.SW-H-^{2b}/SnJ, (NON/LtJ×NZO/HIJ)F1, NZO/HIJ, ALR/LtJ, NON/Ltj, KK.Cg-AALR/LtJ, NON/LtJ, KK.Cg-A^{y}/J, B6.HRS(BKS)-Cpe^{fat}/+, B6.129P2-Gck^{tm/Efr}, B6.V-Lep^{ob}, BKS.Cg-m+/+Lep^{rd}b, and C57BL/6J with Diet Induced Obesity.

Measuring Body Fat Reduction Induced by Candidate Compounds

Once compounds that specifically bind PPARα are identified, their ability to reduce body fat can next be evaluated. Appropriate amounts OEA and/or candidate compounds can be administered to rats via intraperitoneal injection. The OEA and candidate compounds can be formulated in 70% DMSO in sterile saline, 5% Tween 80/5% polyethyleneglycol in sterile saline, or 10% Tween 80/10% ethanol/80% saline. Five mg per kg of OEA serves as the positive control. Amounts of candidate compounds administered range from 1-25 mg per kg. Typically 1, 2, 5, 10, 15, and 20 mg per kg doses of each candidate compound will be administered to different sets of rats to determine which dose is optimal. Injections are given 30 minutes before the animals' principal meal (at approximately 18:00H) for 7-14 days.

The effect of the candidate compound on total body fat can be determined by taking direct measurements of the rat's body fat using skin fold calipers. Skin on the rats' backs, abdomen, chest, front and rear legs can be pinched with calipers to obtain measurements before administration of OEA and/or candidate compounds and every 48 hours during and after administration of OEA and/or candidate compounds. Differences in measurements in at least two of the pinched sites reflect the change in the rat's total body fat.

Measuring Fatty Acid Metabolism Induced by Candidate Compounds

Compounds that specifically bind PPARα can also be assayed for their effect on fatty acid metabolism. The effect of the candidate compound on fatty acid metabolism can be measured by measurements of fatty acid oxidation in primary cultures of liver cells. For instance, hepatocytes can be used to determine the rate of oleate oxidation to ketone bodies and carbon dioxide. In this instance, cells can be isolated from adult rat liver by enzymatic digestion as described by Beynen et al. (1979), Diabetes 28:828. Cells are cultured in suspension and incubated in Krebs-Henseleit's bicarbonate medium supplemented with bovine serum albumin and glucose as described by Guzman & Geelen (1992), Biochem. J. 287:487. The protein concentration of the cultured cells can be determined and cells are seeded in 2 ml media so that 4-6 mg protein per ml is present in the reaction mixture. Cells are incubated for 10 minutes at 37° C. with [¹⁴C]-oleic acid (Amersham), in the presence or absence of 10 μM OEA, reactions are stopped with 200 μl 2M perchloric acid and acid-soluble products are extracted with chloroform/methanol/water (5:1:1, vol/vol/vol). The aqueous phase can be removed and washed twice more. Protein concentration can be determined using a Lowry assay. The rate of oleate conversion into ketone bodies can be expressed as nmol of oleate oxidized per hour per mg protein and can be determined using liquid scintillation counting. Using such methods, OEA enhanced oleate oxidation by 21+-6% (n=4, p<0.01 vs. control incubations by the Student t test).

The following examples are provided to illustrate, and not to limit, the invention.

### EXAMPLES

### Example 1

### Synthesis of Fatty Acid Ethanolamide Compounds, Homologues and Analogs

Methods for the formation of fatty acid ethanolamines from ethanolamines and the corresponding fatty acyl are relatively straight forward and known to one of ordinary skill in the art. For example, fatty acid ethanolamides may be synthesized by reacting a fatty acid or fatty acid chloride with an aminoalcohol as described by Abadjj et al. (Abadji, V., Lin, S. Y., Taha, G., Griffin, G., Stevenson, L. A., Pertwee, R. G. & Makriyannis, A. J. Med. Chem. 37, 1889-1893 (1994)). Fatty acids may be prepared similarly to the procedure of Serdarevich and Carroll (Serdarevich, B. & Carroll, K. K. J. Lipid Res. 7, 277-284 (1966)). Radioactively labeled fatty acid ethanolamides can be prepared by reaction with acyl chlorides (Nu-Check Prep, Elysian, Minn.) with [³H]ethanolamine (10-30 Ci/mmol; American Radiolabeled Chemicals, St. Louis) as described by Desamaud, F., Cadas, H. & Piomelli, D. (1995) J. Biol. Chem. 270, 6030-6035. Compounds can be purified by flash column chromatography or HPLC. Compound identity can be established by use of NMR and/or gas chromatography-mass spectrometry and thin layer chromatography.

Starting reagents and materials may be purchased from Avanti Polar Lipids, Cayman Chemicals (Ann Arbor, Mich.), Nu-Check Prep, Research Biochemicals, or Sigma. Briefly, according to methods taught by Giuffrida, A. et al. (see Giuffrida, A and Piomelli, D. in Lipid Second Messengers (Laycock, S. G. and Rubin, R. P. Eds. pp. 113-133 CRC Press LLC, Boca Raton, Fla.) and Devane et al. (Devane W., Hanus, L. et al., Science 258, 1946-1949 (1992)), unlabeled or labeled fatty acyl ethanolamines can be synthesized by the reaction of the corresponding fatty acyl chlorides with unlabeled or labeled ethanolamine. The fatty acid chorides can be dissolved in dichloromethane (10 mg/ml) and reacted with ethanolamine at −0.4° C. for 15 minutes. The reaction can be quenched by the addition of purified water. After vigorous stirring the phases are allowed to separate. The upper aqueous phase can be discarded. The organic phase can be washed twice with water. These washes remove the unreacted ethanolamine. This method provides a quantitative formation of fatty acyl ethanolamines. The ethanolamines are concentrated to dryness under a stream of nitrogen gas and can be reconstituted in an organic solvent such as dichloromethane at a concentration of 20 mM. The resulting fatty acyl ethanolamine solution can be stored at −20° C. until needed for use.

The chemistry of fatty acid carboxylic acid groups, primary and secondary amines, and primary alcohol groups is well known to one of ordinary skill in the art. Fatty acid ethanolamides having a variety of substituents on the ethanolamine portion thereof can be formed in many ways, but most preferably by starting with the corresponding substituted ethanolamine and fatty acid moieties. Such substituted ethanolamines would include the alkyl aminoethanol ethers and acyl aminoethanol esters as well as secondary akyl ethanol amines. Alternatively, the particular fatty acid ethanolamide can be synthesized from the corresponding fatty acid ethanolamide by the addition of the appropriate substituent groups.

Synthesis of OEA.

Oleoylchloride can be purchased from Nu-Check Prep (Elysian, Minn.) or prepared following standard procedures. Oleoylchloride can be dissolved in dichloromethane (10 mg/ml) and allowed to react with five equivalents of ethanolamine for 15 min. at 0-4° C. The reaction can be stopped by the addition of purified water. After vigorous stirring and phase separation, the upper aqueous phase can be discarded and the organic phase washed twice with water to remove non-reacted ethanolamine. The resulting OEA can be concentrated to dryness under a N₂ stream, reconstituted in chloroform at 20 mM, and stored at −20° C. until use.

### Example 2

### Test Methods, Physiology and Pharmacological Activity of OEA-Like Compounds and/or OEA-Like Modulators

Animals. Male Wistar rats (200-350 g) were used. Procedures should meet NIH guidelines detailed in the Guide for the Care and Use of Laboratory Animals, and the European Communities directive 86/609/EEC regulating animal research.

Chemicals. FAEs and [²H₄] FAEs were synthesized in the laboratory (Giuffrida et al., “Lipid Second Messengers” (ed. Laychock, S. G. & Rubin, R. P.) 113-133 (CRC Press LLC, Boca Raton, Fla., 1998)); 1,2-dioleyl-sn-glycero-phosphoethanolamine-N-oleyl was purchased from Avanti Polar Lipids (Alabaster, Ala.); SR141716A was provided by RBI (Natick, Mass.) as part of the Chemical Synthesis Program of the NIMH (N01MH30003); SR144528 was a generous gift of Sanofi Recherche; all other drugs were from Tocris (Ballwin, Mo.) or Sigma (Saint Louis, Mo.). FAE were dissolved in dimethylsulphoxide (DMSO) and administered in 70% DMSO in sterile saline (acute treatments) or 5% Tween 80/5% propylenglycol in sterile saline (subchronic treatments) (1 ml per kg, i.p.). Capsaicin was administered in 10% Tween 80/10% ethanol/80% saline; SR141716A, SR144528, CCK-8 and CP-93129 in 5% Tween 80/5% propylenglycol/90% saline (1 ml per kg, i.p.).

Enzyme assays. In all biochemical experiments, rats were killed and tissues collected between 1400 and 1600 h, after varying periods of food deprivation. Microsome fractions were prepared as described (Desamaud et al., J. Biol. Chem., 270:6030-6035 (1995)). NAT assays were performed using 1,2-di[¹⁴C]palmityl-sn-glycerophosphocholine as a substrate (108 mCi/mmol, Amersham, Piscataway, N.J.) (Cadas et al., H., J. Neurosci., 17:1226-1242 (1997)). FAAH assays were performed according to (Desamaud et al., J. Biol. Chem., 270:6030-6035 (1995)), except that [³H]anandamide (arachidonyl-[1-³H]ethanolamide; 60 Ci/mmol; ARC, St. Louis, Mo.) was included as a substrate and radioactivity was measured in the aqueous phase after chloroform extraction.

HPLC/MS analyses. Plasma was prepared from blood obtained by cardiac puncture (Giuffrida et al., Anal. Biochem., 280:87-93 (2000)) and CSF was collected from the cisterna magna using a 27G 1/2 needle (Precisionglide, USA). FAEs and NAPE were extracted from tissues with methanol/chloroform and fractionated by column chromatography (Giuffrida et al., “Lipid Second Messengers” (ed. Laychock, S. G. & Rubin, R. P.) 113-133 (CRC Press LLC, Boca Raton, Fla., 1998)). FAEs were quantified by HPLC/MS, using an isotope dilution method (Giuffrida et al., Anal. Biochem., 280:87-93 (2000)). Individual NAPE species were identified and quantified by HPLC/MS, using an external standard method (Calignano et al., Nature, 408:96-101 (2000)).

Blood chemistry. Plasma β-hydroxybutyrate and glycerol were measured using commercial kits (Sigma, St. Louis, Mo.). Plasma prolactin, corticosterone and luteinizing hormone were quantified by radioimmunoassay (Navarro et al., Neuroreport, 8:491-496 (1997)).

Feeding experiments. Acute experiments. Food intake was measured in 24-h food-deprived rats (Navarro et al., J. Neurochem., 67:1982-1991 (1996)), administering drugs 15 min before food presentation. Subchronic experiments. Ad libitum fed rats received vehicle injections for three days. On day four, the animals were divided in two equal groups and gave them daily injections of vehicle or OEA (5 mg per kg at 1900 h) for 7 consecutive days, while measuring body weight, food intake and water intake.

Conditioned taste aversion. Rats were water-deprived for 24 h and then accustomed to drink from a graded bottle during a 30-min test period for four days. On day five, water was substituted with a 0.1% saccharin solution and, 30 min later, the animals received injections of vehicle, OEA (20 mg per kg) or lithium chloride (0.4 M, 7.5 ml per kg). During the following two days, water consumption was recorded over 30-min test periods. The animals were then presented with water or saccharin, and drinking measured.

Operant responses for food. Rats were trained to lever press for food on a fixed ratio 1 (FR1) schedule of reinforcement, while food-restricted at 20 g of chow per rat per day (Rodriguez de Fonseca et al., Acta Pharmacol. Sin., 20:1109-1114 (1999)). Once stable responding was achieved, the animals were trained to acquire an FR5, time out 2-min schedule of food reinforcement and kept in limited access to food. When a stable baseline was obtained, the animals were used to test the effects of vehicle or OEA (1, 5 or 20 mg per kg) administered 15 min before lever presentation. Test duration was 60 min.

Other behavioral assays. The elevated plus maze test was conducted as described (Navarro et al., Neuroreport, 8:491-496 (1997)) after the administration of vehicle or OEA (20 mg per kg, i.p.). Horizontal activity in an open field (Beltramo et al., J. Neurosci., 20:3401-3407 (2000)) and pain threshold in the hot plate test (55° C.) (Beltramo et al., Science, 277:1094-1097 (1997)) were measured 15 min after injection of vehicle or OEA (20 mg per kg). Rectal temperature was measured using a digital thermometer (Martin-Calderón et al., Eur. J. Pharmacol., 344:77-86. (1998)).

In situ hybridization. Rats were accustomed to the handling and injection procedure for five days. On day six, vehicle or drug OEA (10 mg per kg, i.p.), or oleic acid (10 mg per kg) was administered, and the rats killed 60 min later by decapitation under anesthesia. In situ hybridization analyses were conducted using ³⁵S-labeled cRNA probes for c-fos (Guthrie et al., Proc. Natl. Acad. Sci. U.S.A., 90:3329-3333 (1993)) and choline acetyl transferase (CHAT) (Lauterborn et al., Brain Res. Mol. Brain Res., 17:59-69 (1993)). Average hybridization densities were determined from at least three tissue sections per rat. Statistical significance was evaluated using one-way analysis of variance (ANOVA) followed by the Tukey-Kramer post-hoc test for paired comparisons.

Data analysis. Results are expressed as mean±s.e.m of n separate experiments. The significance of differences among groups was evaluated using ANOVA followed by a Student-Newman-Keuls post hoc test, unless indicated otherwise.

A. Effects of Starvation on OEA and Other FAE Levels in the Rat.

In one embodiment, the invention provides methods of treatment wherein individuals needing to lose weight and/or body fat are tested for OEA levels before and/or during fasting. Individuals with low levels of OEA prior to or in response to fasting are particularly then targeted for OEA treatment.

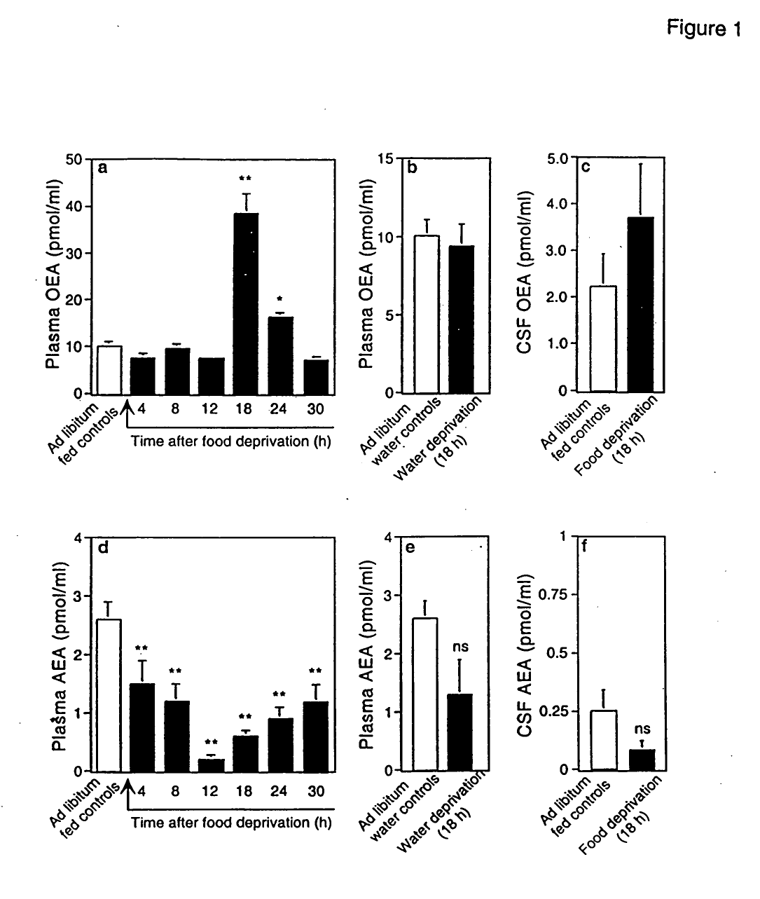

Rats were deprived of food while periodically measuring FAE levels in cardiac blood by high-performance liquid chromatography (HPLC) coupled to electrospray mass spectrometry (MS). Plasma OEA remained at baseline levels for the first 12 h of fasting, markedly increased at 18-24 h, and returned to normal at 30 h (FIG. 1a). No such effect was observed following water deprivation (FIG. 1b) or application of stressors such as restraint immobilization and lipopolysaccharide (LPS) administration [in pmol per ml; 10.3±0.8; 60 min after a 15-min immobilization, 8.4±1.6; 60 min after LPS injection (1 mg per kg), 7.0±0.7; n=6-9]. Plasma PEA was not significantly affected by any of these treatments (data not shown), whereas anandamide decreased rapidly upon food removal, remaining lower than baseline for the entire duration of the experiment (FIG. 1d). Anandamide levels also declined after immobilization (in pmol per ml; control, 3.6±0.4; immobilization, 1.1±0.5; n=7-8; P<0.01), LPS treatment (control, 2.0±0.5; LPS, 0.2±0.2; n=6; P<0.01) and, though not significantly, water deprivation (FIG. 1e). These results indicate that circulating OEA levels increase transiently during starvation. This response is selective for OEA over anandamide and other FAEs, and coincides temporally with the rise in blood glycerol and β-hydroxybutyrate (Table 1), which signals the shift of energy metabolism from carbohydrates to fatty acids as primary fuel (Cahill, G. F., Clin. EndocrinoL Metab., 5:397-415 (1976)).

TABLE 1
Plasma level of β-hydroxybutyrate (β-HBA) and glycerol
in fasting rats.

| | β-HBA | Glycerol |
|---|---|---|
| Free feeding | 1.2 ± 0.4 | 4.6 ± 0.9 |
| 2 h fasted | 1.2 ± 0.2 | 5.3 ± 0.6 |
| 4 h fasted | 0.8 ± 0.1 | 9.1 ± 1.8 |
| 8 h fasted | 1.3 ± 0.2 | 6.3 ± 0.4 |
| 12 h fasted | 4.6 ± 0.8* | 7.6 ± 1.0 |
| 18 h fasted | 6.8 ± 0.4* | 8.4 ± 0.4* |
| 24 h fasted | 9.1 ± 1.2* | 8.4 ± 0.3* |

Concentrations are expressed in mg per dl.
*P < 0.05, n = 3 per group.

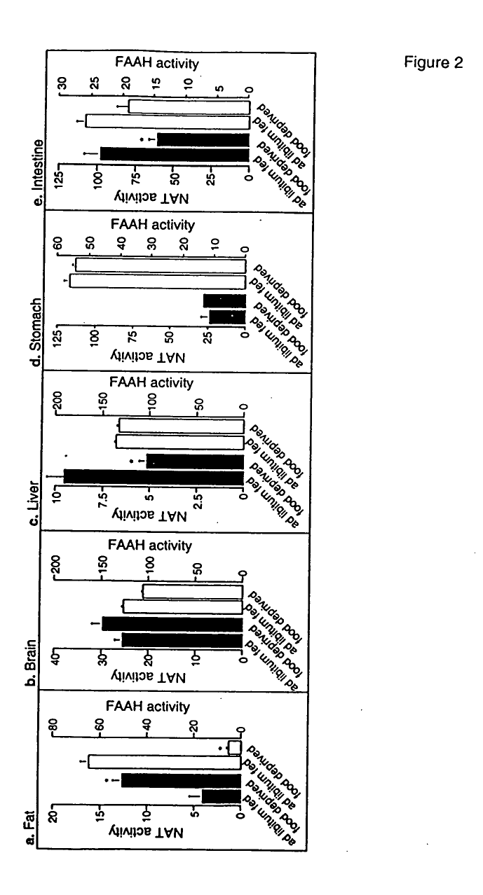

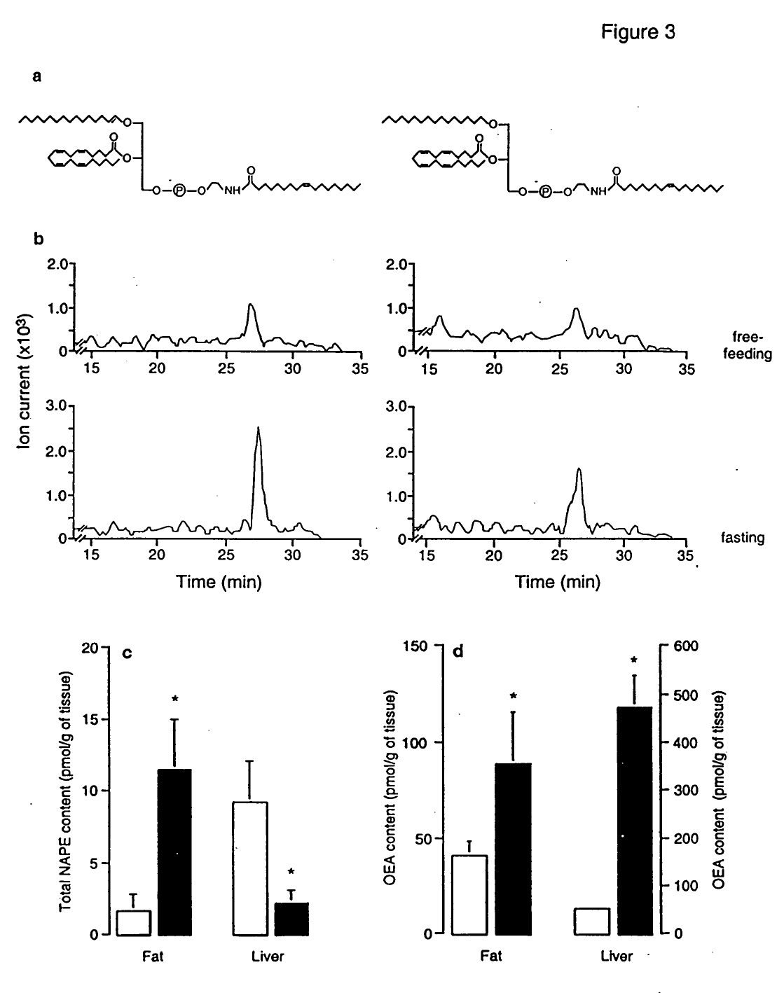

OEA levels in cerebrospinal fluid were not significantly affected by food deprivation (FIG. 1c), implying that the surge in plasma OEA may originate outside the CNS. To test this hypothesis, the impact of starvation on OEA metabolism in various rat tissues was investigated. The biochemical route by which animal cells produce and degrade OEA and other FAEs is thought to comprise three key enzymatic steps. Calcium ion-stimulated NAT activity transfers a fatty acid group from the sn-1 position of a donor phospholipid to the primary amine of phosphatidylethanolamine, producing NAPE2 (Schmid et al., Chem. Phys. Lipids, 80:133-142 (1996); Piomelli et al., Neurobiol. Dis., 5:462-473 (1998)). Cleavage of the distal phosphodiester bond in NAPE by an unknown phospholipase D generates FAEs (Schmid et al., Chem. Phys. Lipids, 80:133-142 (1996); Piomelli et al., Neurobiol. Dis., 5:462-473 (1998)), which are eventually broken down to fatty acid and ethanolamine by an intracellular fatty acid amide hydrolase (FAAH) (Schmid et al., J. Biol. Chem., 260:14145-14149 (1985); Cravatt et al., Nature, 384:83-87 (1996)). Food deprivation (18 h) was accompanied by a marked increase in NAT activity in white adipose tissue (FIG. 2a), but not in the brain, stomach or kidney (FIG. 2b,d and data not shown). In liver, intestines and skeletal muscle, NAT activity was reduced by fast (FIG. 2c,d and data not shown). These enzymatic changes were paralleled by corresponding alterations in NAPE tissue content. Several molecular species of NAPE are present in rat tissues, including the OEA precursors alk-1-palmitoenyl-2-arachidonyl-sn-glycero-phosphoethanolamine-N-oleyl (NAPE 1; FIG. 3a) and alk-1-palmityl-2-arachidonyl-sn-glycero-phosphoethanolamine-N-oleyl (NAPE 2; FIG. 3a); and the PEA precursor alk-1-palmityl-2-arachidonyl-sn-glycero-phosphoethanolamine-N-palmityl (not shown). In agreement with NAT activity measurements, food deprivation increased NAPE content in fat, and decreased it in liver (FIG. 3b,c).

Since NAPE biosynthesis and FAE formation are tightly coupled processes (Cadas et al., H., J. Neurosci., 17:1226-1242 (1997)), one might expect starvation to augment the levels of OEA and other FAEs in adipose, but not in other tissues. Accordingly, fat from starved rats contained more OEA and PEA than did fat from free-feeding controls (FIG. 3d and data not shown), whereas no such difference was seen in the brain, stomach, and intestines (data not shown). Contrary to our expectation, however, the liver content of OEA and PEA was also higher in food-deprived than in free-feeding rats (FIG. 3d and data not shown). This discordance may be due to an accumulation of FAEs by the liver, which is consistent with the postulated roles of this organ in FAE recapture and metabolism (Bachur et al., J. Biol. Chem., 240:1019-1024 (1965); Schmid et al., J. Biol. Chem., 260:14145-14149 (1985)).

The hydrolysis to fatty acid and ethanolamine, catalyzed by FAAH, is a key step in FAE degradation (Bachur et al., J. Biol. Chem., 240:1019-1024 (1965); Schmid et al., J. Biol. Chem., 260:14145-14149 (1985); Cravatt et al., Nature, 384:83-87 (1996); Desarnaud et al., J. Biol. Chem., 270:6030-6035 (1995)). Food deprivation profoundly reduced FAAH activity in adipose membranes, but had no effect on FAAH activity in the brain, liver, stomach, intestines, kidney and skeletal muscle (FIG. 2a-e and data not shown). Thus, food deprivation may increase the levels of OEA and other FAEs in white fat in two synergistic ways, which are mechanistically distinct from other reactions occurring during lipolysis: stimulation of NAT activity may lead to increase the biosynthesis of NAPE and FAEs, while inhibition of FAAH activity may prolong the life span of newly synthesized FAEs. Although several tissues may contribute to the normal levels of OEA in the bloodstream, the dynamic biochemical changes observed in fat underscore the crucial role of this tissue in generating OEA during starvation.

B. Suppression of Food Intake by OEA and Other FAEs.

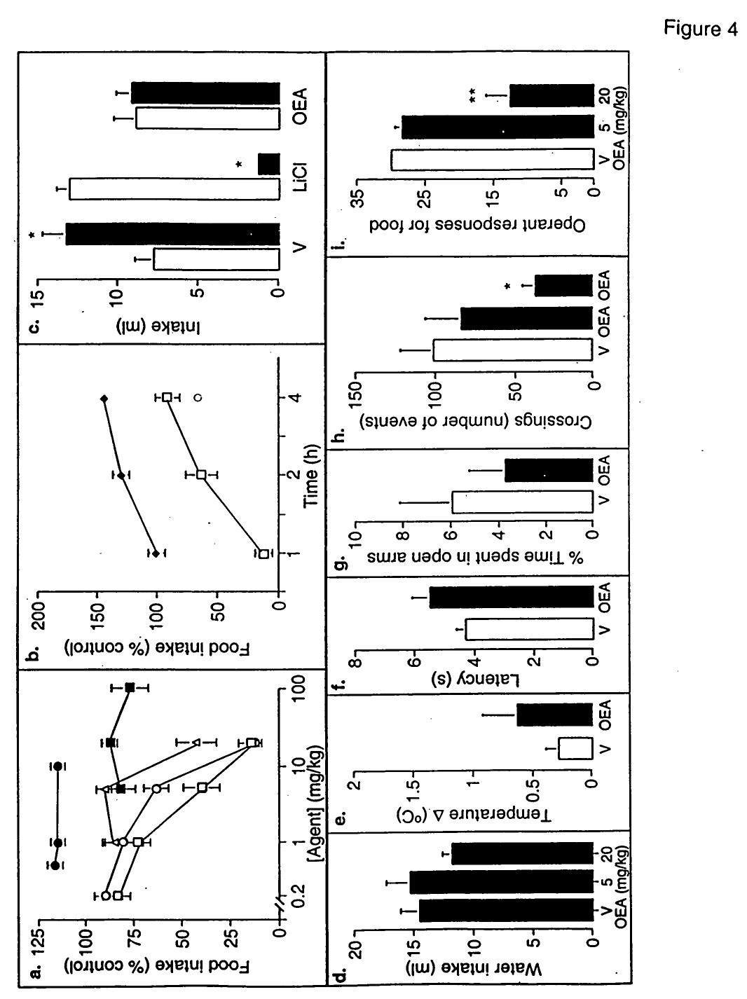

The effects of systemically administered OEA or an OEA-like compound or OEA-like modulator on food intake in rats can be assessed using a 24 h fast. In this system, OEA caused a dose- and time-dependent suppression of food intake (FIG. 4a,b) in rats given access to food after fasting. To define the selectivity of this response, various OEA analogs were evaluated for their ability to produce hypophagia.

Anandamide and oleic acid had no effect.

Palmitoylethanolamide was active but significantly less potent than OEA.

Elaidylethanolamide (an unnatural OEA analog) was similar in potency to OEA (FIG. 4a).

These results indicate that OEA reduces eating in a structurally selective manner and that other fatty acid ethanolamide-like compounds can be identified for use according to the invention.

C. Specificity Over Cannabinoid Receptor Activators.

The molecular requisites for OEA hypophagia appear to be distinct from those involved in the interaction of anandamide with its known cannabinoid targets (Khanolkar et al., Life Sci., 65:607-616 (1999)). Cannabinoid receptor antagonists did not affect OEA hypophagia in vivo, and OEA did not displace cannabinoid binding to rat brain membranes in vitro. Thus, despite its structural and biogenetic relationships with anandamide, OEA acts differently and does not so depend on the endogenous cannabinoid system to produce anorexia.

D. Sustained Body Weight Reduction

In some embodiments, the OEA-like compounds and OEA-like modulators of the instant invention provide for a sustained fat reduction or body weight reduction upon prolonged administration to mammals. This effect can be advantageous as a variety of drugs suppress eating after acute administration, but fail to do so when treatment is prolonged (Blundell, J., Trends Pharmacol. Sci., 12:147-157 (1991)).

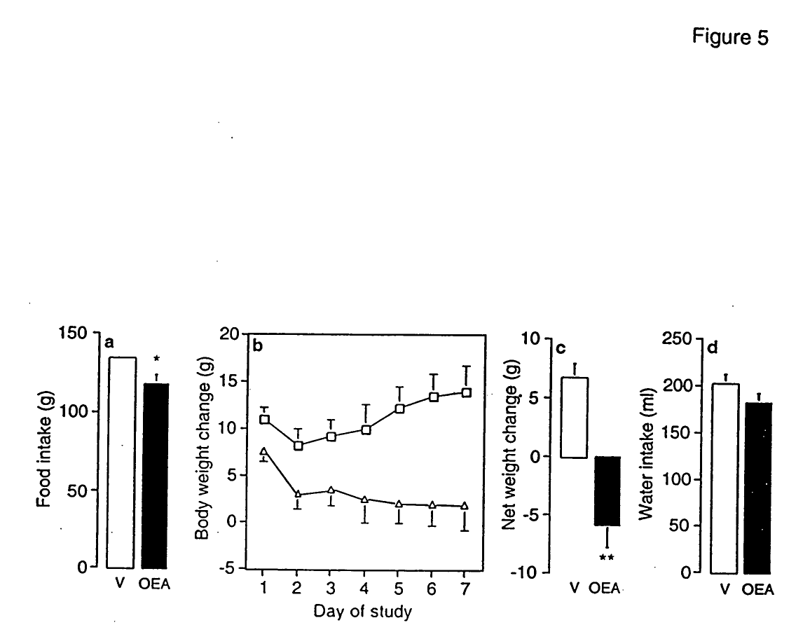

In this example, OEA was subchronically administered to rats. Daily injections of OEA (5 mg per kg, i.p.) for seven days resulted in a small, but significant decrease in cumulative food intake (FIG. 5a), which was accompanied by a profound inhibition of weight gain (FIG. 5b, c). OEA did not affect water intake (FIG. 5d). Without being wed to theory, the impact of OEA on body weight may only be partially explained by its moderate reduction of food consumption indicating that other factors, such as stimulation of energy expenditure or inhibition of energy accumulation, may contribute to this effect.

E. FAE's May Have a Peripheral Site of Action

In one of its aspects, the invention provides OEA-like compounds and OEA-like modulators having a peripheral site of action. Such a site can be advantageous in reducing the likelihood of central nervous system side effects.

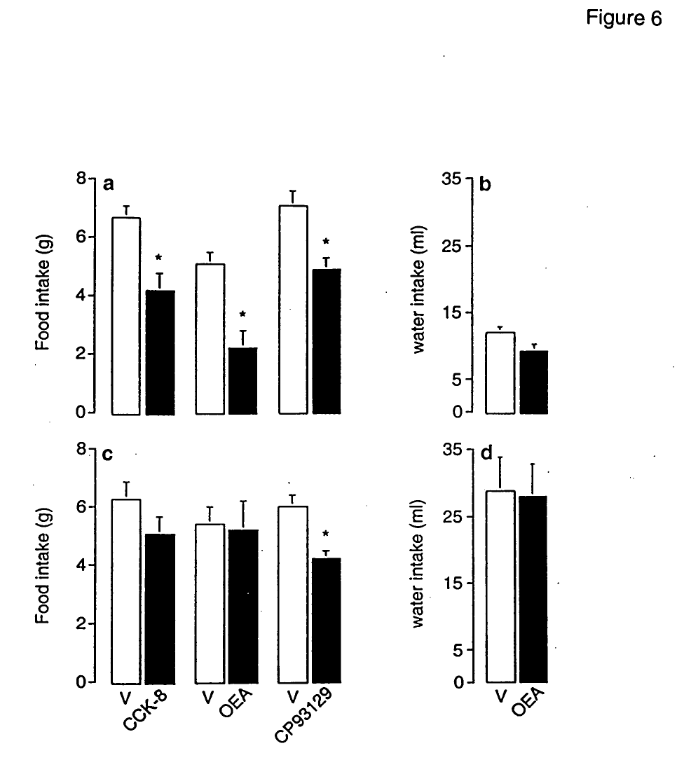

Though potent when administered peripherally, OEA was ineffective after direct injection into the brain ventricles (Table 2), suggesting that the primary sites of action of this compound might be located outside the CNS. As a further demonstration, sensory fibers in the vagus and other peripheral nerves were chemically destroyed by treating adult rats with the neurotoxin, capsaicin (Kaneko et al., Am. J. Physiol., 275:G1056-G1062 (1998)). Capsaicin-treated rats failed to respond to peripherally administered cholecystokinin-8 (CCK-8) (FIG. 6,a,c), drank more water than controls (FIG. 6b,d) and lost the corneal chemosensory reflex (data not shown), three indications that the neurotoxin had destroyed sensory afferents (MacLean, D. B., Regul. Pept., 11:321-333 (1985); Ritter et al., Am. J. Physiol., 248:R501-R504 (1985); Curtis et al., Am. J. Physiol., 272:R704-R709 (1997)). Treated animals also failed to respond to OEA (10 mg per kg, i.p.), but responded normally to the compound CP-93129, which targets 5-HT1B receptors in the CNS (FIG. 6a,c) (Lee et al., Psychopharmacology, 136:304-307 (1998)). Without being wed to theory, these findings support the hypothesis that OEA causes hypophagia by acting at a peripheral site, and that sensory fibers are required for this effect.

TABLE 2
Effects of intracerebroventricular OEA on food intake.

| | 60 min | 120 min | 240 min |
|---|---|---|---|
| vehicle | 5.8 ± 0.6 | 8.0 ± 0.5 | 9.5 ± 0.5 |
| OEA 0.4 μg | 4.8 ± 0.4 | 6.6 ± 0.4 | 8.4 ± 0.4 |
| OEA 2 μg | 4.9 ± 0.4 | 6.6 ± 0.6 | 8.7 ± 0.5 |
| OEA 10 μg | 5.9 ± 0.2 | 8.1 ± 0.4 | 9.6 ± 0.7 |

OEA(μg per animal) or vehicle (DMSO, 5 μl) was administered to 24 h food-deprived rats 15 min before food presentation. n = 12 per group.

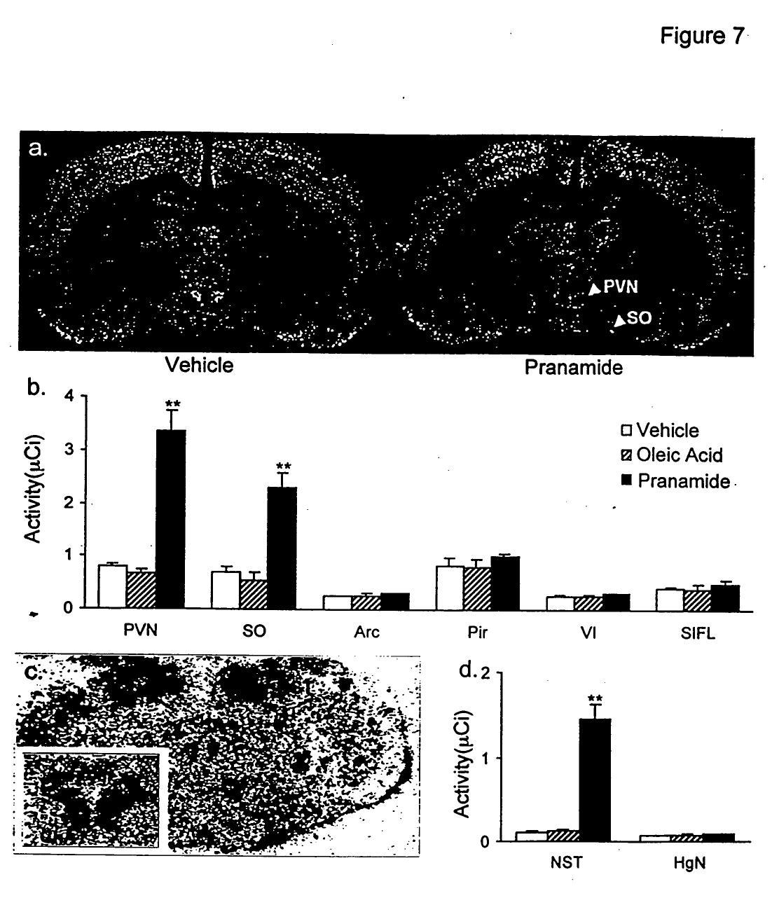

The compounds of the invention may use peripheral sensory inputs to suppress appetite. Peripheral sensory inputs related to appetite suppression recruit several CNS structures, which include the nucleus of the solitary tract (NST) in the brainstem and the arcuate and paraventricular (PVN) nuclei in the hypothalamus (Schwartz et al., Nature, 404:661-671 (2000)). To identify the brain pathways engaged during OEA-induced hypophagia, mRNA levels for the activity regulated gene c-fos (Curran et al., Oncogene, 2:79-84 (1987)) were mapped by in situ hybridization after systemic administration of OEA, oleic acid or vehicle. When compared to controls, OEA (10 mg per kg, i.p.) evoked a highly localized increase in c-fos mRNA levels in the PVN, supraoptic nucleus (FIG. 7a) and NST (FIG. 7c). This enhancement was specific to these areas, insofar as c-fos expression in other brain regions was not significantly affected by OEA treatment (FIG. 7b,d). The finding that OEA stimulates c-fos mRNA expression in the NST (which processes vagal sensory inputs to the CNS) and the PVN (a primary site for the orchestration of central catabolic signals) (Schwartz et al., Nature, 404:661-671 (2000)), is consistent with a physiological role for this lipid as a peripheral mediator of anorexia.

OEA may reduce eating by inducing a non-specific state of behavioral suppression. If this is the case, OEA should cause conditioned taste aversion, which can be readily provoked in rats by a number of noxious substances (Green et al., Science, 173:749-751 (1971)), including lithium chloride (FIG. 4c). However, a maximal dose of OEA (20 mg per kg, i.p.) had little effect in this assay (FIG. 4c), suggesting that the compound may not be aversive. Several additional observations support the behavioral specificity of OEA. OEA did not alter water intake, body temperature, pain threshold (FIG. 4d-f), or activity of the hypothalamus-pituitary-adrenal (HPA) axis (Table 3). Moreover, OEA did not produce anxiety-like symptoms (FIG. 4g) and, though it reduced motor activity and operant responses for food, it did so at a dose that was substantially higher than those required to produce hypophagia (FIG. 4h-i). This pharmacological profile differentiates OEA from other appetite suppressants such as amphetamine and glucagon-like peptide 1 (whose effects often include aversion, hyperactivity, anxiety and activation of the HPA axis) and from the endogenous cannabinoid anandamide (which stimulates food intake in partially satiated animals, increases pain threshold, decreases body temperature and activates the HPA axis) (Pertwee, R. G., Exp. Opin. Invest. Drugs, 9:1553-1571 (2000)).

TABLE 3
Effects of OEA on plasma hormone levels.

| | B | PRL | LH |
|---|---|---|---|
| vehicle | 212 ± 24 | 10.8 ± 2.7 | 5.3 ± 0.9 |
| OEA 20 | 280 ± 61 | 8.2 ± 3.2 | 6.2 ± 1.5 |

In Table 2, plasma corticosterone (B), prolactin (PRL) and luteinizing hormone (LH) levels were measured by radioimmunoassay in plasma samples collected 60 min after injection of vehicle or OEA (prana, in mg per kg, i.p.) and are expressed in ng per ml. n = 6-9 per group.

OEA elicits hypophagia at physiologically relevant doses. 1 hr after administration of a half-maximally effective dose (5 mg per kg, i.p.), circulating OEA levels (16.1±2.6 pmol per ml) were significantly higher than baseline (10.1±1.1; P<0.05, Student's t test; n=5), but below those measured in 18-h food-deprived animals (FIG. 1a). Thus, the concentrations reached by OEA in blood during starvation can be sufficient to elicit notable behavioral responses.

F. Identifying Body Fat Reducing Compounds of the Invention.

The following illustrates how to identify appetite suppressors using OEA as a positive control. In particular, the measurement of body fat reduction and fatty acid oxidation are discussed.

The ability of an OEA-like compound or OEA-like modulator to reduce body fat can be evaluated by a number of methods. For example, appropriate amounts OEA and/or candidate compounds are administered to rats via intraperitoneal injection. The OEA and candidate compounds can be formulated in 70% DMSO in sterile saline, 5% Tween 80/5% polyethyleneglycol in sterile saline, or 10% Tween 80/10% ethanol/80% saline. Five mg per kg of OEA can be used as the positive control. Amounts of candidate compounds administered may range, for instance, from 1-25 mg per kg. Typically 1, 2, 5, 10, 15, and 20 mg per kg doses of each candidate compound can be administered to different sets of rats to determine which dose is optimal. Injections may be given 30 minutes before the animals' principal meal for 7-14 days.

The effect of the candidate compound on total body fat can be determined by taking direct measurements of the rat's body fat using skin fold calipers. Skin on the rats' backs, abdomen, chest, front and rear legs can be pinched with calipers to obtain measurements before administration of OEA and/or candidate compounds and every 48 hours during and after administration of OEA and/or candidate compounds. Differences in measurements in at least two of the pinched sites reflect the change in the rat's total body fat.

OEA-like compounds and modulators can be used to modulate fat metabolism. Such compounds can also be assayed for their effect on fatty acid metabolism. The effect of the candidate compound on fatty acid metabolism can be measured by measurements of fatty acid oxidation in primary cultures of liver cells. Hepatocytes may be used to determine the rate of oleate oxidation to ketone bodies and carbon dioxide. Such cells can be isolated from adult rat liver by enzymatic digestion as described by Beynen et al. in Diabetes 28:828 (1979). Cells typically are cultured in suspension and incubated in Krebs-Henseleit's bicarbonate medium supplemented with bovine serum albumin and glucose as described by Guzman & Geelen, Biochem. J. 287:487(1992). The protein concentration of the cultured cells can be determined and cells seeded in 2 ml media so that 4-6 mg protein per ml is present in the reaction mixture. Cells can be incubated for 10 minutes at 37° C. with [¹⁴C]-oleic acid (Amersham), in the presence or absence of 10 μM OEA, reactions may be stopped with 200 μl 2M perchloric acid and acid-soluble products extracted with chloroform/methanol/water (5:1:1, vol:vol:vol). The aqueous phase can be removed and washed twice more. Protein concentration can be determined using a Lowry assay. The rate of oleate conversion into ketone bodies may be expressed as nmol of oleate oxidized per hour per mg protein and may be determined using liquid scintillation counting. Accordingly, OEA enhances oleate oxidation by 21+-6% (n=4, p<0.01 vs. control incubations by the Student t test).

G. Effect of OEA on Fatty Acid Metabolism.

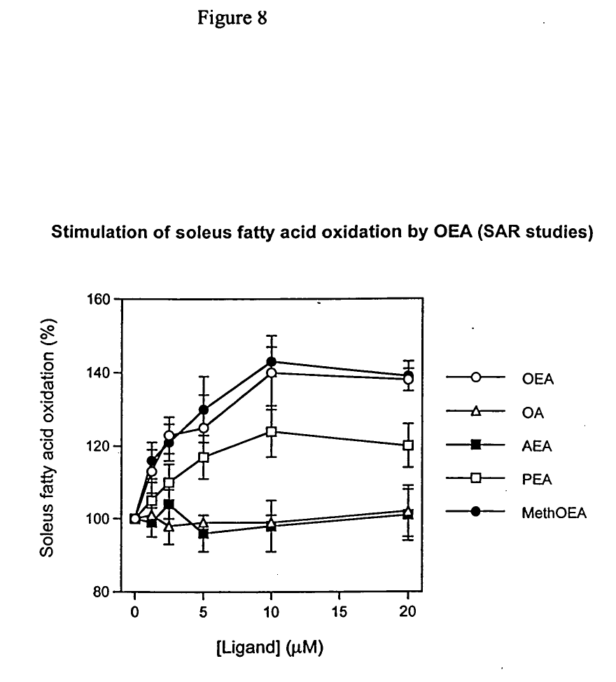

This example illustrates the effect of OEA on fat metabolism and methods for studying the same. Oleoylethanolamide (OEA) decreases body weight not only by suppressing appetite, but also by possibly enhancing body fat catabolism. The effects of OEA on fatty acid oxidation in major body-fat burning tissues (soleus muscle, liver, cultured cardiac myocytes and astrocytes) was examined. OEA significantly stimulates fatty acid oxidation in primary cultures of liver, skeletal muscle (soleus) and heart cells, whereas it has no effect in brain-derived astroglial cell cultures. In addition, OEA induces a significant mobilization of triacylglycerol stores from primary white adipose tissue cells. Table 4 details the methods and effects of OEA on fatty acid oxidation in these cells. Structure-activity relationship experiments provide evidence that the effect of OEA on skeletal muscle fatty acid oxidation is specific (FIG. 8). Thus, the effects of OEA are mimicked by the hydrolysis-resistant homologue methyl-OEA and -only partially by palmitoylethanolamide (PEA), but not by arachidonylethanolamide (AEA) or oleic acid (OA). In short, these results show that lipid oxidation and mobilization are enhanced by OEA, and that the effects of OEA are restricted to peripheral sites.

TABLE 4

| Cell/tissue | Hepatocyte | Soleus muscle | Cardiomyocyte | Astrocyte | Adipocyte |
|---|---|---|---|---|---|
| Origin | Adult rat liver | Adult rat hind | Newborn rat | Newborn rat | Adult rat |
| | | limb | heart | brain cortex | epididymus |
| Isolation | Enzymatic | Dissection | Enzymatic | Enzymatic | Enzymatic |
| procedure | digestion | (Chiasson, | digestion (Flink | digestion | digestion |
| | (Beynen et al., | 1980) | et al., 1992) | (McCarthy & | (Rodbell, |
| | 1979) | | | De Vellis, | 1964) |
| | | | | 1980) | |
| Type of | Cell | Tissue | Cell monolayer | Cell | Cell |
| culture | suspension | suspension | | monolayer | suspension |
| Incubation | Krebs- | Krebs-Henseleit | High-glucose | Hams | Krebs- |
| medium | Henseleit | Hepes plus | DMEM plus | F12/DMEM | Henseleit |
| | bicarbonate | BSA and | BSA | plus insulin, | Hepes plus |
| | plus BSA and | glucose | (Wu et al., | transferrin, | BSA and |
| | glucose | (Fruebis et al., | 2000) | progesterone, | glucose |
| | (Guzman & | 2001) | | putrescine | (Rodbell, |
| | Geelen, 1992) | | | and selenite | 1965) |
| | | | | (Blazquez et | |
| | | | | al., 1998) | |
| Metabolic | [¹⁴C]oleate | [¹⁴C]oleate | [¹⁴C]oleate | [¹⁴C]oleate | Lypolysis |
| parameter | oxidation to | oxidation to | oxidation to | oxidation to | (glycerol |
| | ketone bodies | CO₂ (Fruebis et | CO₂ (Blazquez | ketone bodies | release) |
| | (Guzman & | al., 2001) | et al., 1998) | (Blazquez et | (Serradeil- |
| | Geelen, 1992) | | | al., 1998) | Le Gal et |
| | | | | | al., 2000) |
| Incubation | 10 | 30 | 30 | 30 | 30 |
| time (min) | | | | | |
| Stimulatory | 21 ± 6 (n = 4) | 36 ± 10 (n = 4) | 37 ± 9 (n = 3) | 2 ± 6 (n = 3) | 38 ± 16 (n = 3) |
| effect of 10 μM | | | | | |
| OEA | | | | | |
| (%) | | | | | |
| Statistical | P < 0.01 | P < 0.01 | P < 0.01 | Non | P < 0.01 |
| significance | | | | significant | |
| vs. control | | | | | |
| References cited: Beynen AC et al., Diabetes 28: 828-835 (1979); Blazquez C et al., J Neurochem 71: 1597-1606 (1998); Chiasson RB “Laboratory Anatomy of the White Rat” WCB, Dubuque, Iowa (1980); Funk IL et al., J Biol Chem 267: 9917-9924 (1992); Fruebis J et al., Proc Natl Acad Sci USA 98: 2005-2010 (2001); Guzman M et al., Biochem J 287: 487-492 (1992); McCarthy KD et al., J Cell Biol 85: 890-902 (1980); Rodbell M J Biol Chem | | | | | |
| # 239: 375-380 (1964); Rodbell M Ann NY Acad Sci 131: 302-314 (1965); Serradeil-Le Gal C et al., FEBS Left 475: 150-156 (2000); Wu W et al., J Biol Chem 275: 40133-40119 (2000). | | | | | |

H. Role of Endogenous OEA in the Intestines.

The impact of feeding on intestinal OEA biosynthesis was studied. High performance liquid chromatography/mass spectrometry analyses revealed that small intestinal tissue from free-feeding rats contains substantial amounts of OEA (354±86 pmol per g, n=3). Intestinal OEA levels were markedly decreased after food deprivation, but returned to baseline after refeeding. By contrast, no such changes were observed in stomach (in pmol per g; control, 210±20; starvation, 238±84; starvation/refeeding, 239±60, n=3). Variations in intestinal OEA levels were accompanied by parallel alterations in NAT activity, which participates in OEA formation, but not in fatty acid amide hydrolase activity, which catalyzes OEA hydrolysis. These findings suggest that starvation and feeding reciprocally regulate OEA biosynthesis in small intestine. In agreement with an intra-abdominal source of OEA, plasma OEA levels in starved rats were found to be higher in portal than in caval blood (in pmol per ml; porta, 14.6±1.8; cava, 10.3±2.8; n=5). The contribution of other intra-abdominal tissues to OEA formation cannot be excluded at present. These results suggest many interventions to utilize the OEA systems in feeding behavior. According to this model, food intake may stimulate NAT activity enhancing OEA biosynthesis in the small intestine and possibly other intra-abdominal tissues. Newly produced OEA may activate local, sensory fibers, which may in turn inhibit feeding by engaging brain structures such as the NST and PVN.

The above results for Example 2 reveal an unexpected role for OEA in the peripheral regulation of feeding, and provide a framework to develop novel medicines for reducing body weight or body fat, for preventing body weight gain or body fat increase, for suppressing appetite or reducing food seeking behavior, or food intake, and for the treating eating disorders, overweight, or obesity. These medicines would include not only OEA analogues and homologues but also agents which control OEA levels by acting upon the OEA formation and hydrolyzing systems and enzymes as disclosed above.

### Example 3

### PPAR Modulation by OEA-Like Compounds and OEA-Like Modulators Methods, Physiology and Pharmacology

Chemicals

GW 7647 {2-(4-{2-[3-Cyclohexyl-1-(4-cyclohexyl-butyl)-ureido]-ethyl}-phenylsulfanyl)-2-methyl-propionic acid was synthesized as follows. Phenethylamine was reacted with 4-cyclohexyl-butyric acid in the presence of diisopropylcarbodiimide and hydroxybenzotriazole (HOBT) in CH₂Cl₂. The resulting amide was treated with chlorosulfonic acid and PCI₅ to obtain 4-[2-(4-Cyclohexyl-butyrylamino)-ethyl]-benzenesulfonyl chloride, which was reduced (zinc dust/NaOAc/Ac₂O/glacial AcOH), to give thioacetic acid S-{4-[2-(4-cyclohexyl-butyrylamino)-ethyl]-phenyl} ester, the reaction of which with 2-bromo-2-methyl-propionic acid tert-butyl ester under strong basic condition afforded 2-{4-[2-(4-cyclohexyl-butyrylamino)-ethyl]-phenylsulfanyl}-2-methyl-propionic acid tert-butyl ester. This intermediate was then used in the synthetic route reported by Brown et al (Brown et al., 2000), leading to the title compound.

GW501516 [2-Methyl-4-[4-methyl-2-(4-trifluoromethyl-phenyl)-thiazol-5-ylmethylsulfanyl]-phenoxy}-acetic acid was synthesized via basic hydrolysis of the corresponding ethyl ester, prepared by coupling 5-chloromethyl-4-methyl-2-(4-trifluoromethyl-phenyl)-thiazole with (4-mercapto-2-methyl-phenoxy)-acetic acid ethyl ester (Chao et al., 2001). To prepare the latter, o-tolyloxy-acetic acid ethyl ester was treated with chlorosulfonic acid to give (4-chlorosulfonyl-2-methyl-phenoxy)-acetic acid ethyl ester (synthesized). Reduction to (4-acetylsulfanyl-2-methyl-phenoxy)-acetic acid ethyl ester (zinc dust/NaOAc/Ac₂O/glacial AcOH), followed by hydrolysis under mild basic conditions (pyrrolidine in ethanol) yielded the desired intermediate (4-mercapto-2-methyl-phenoxy)-acetic acid ethyl ester.

OEA and other fatty acid ethanolamides can be prepared as described in Giuffrida et al., 2000). All other chemicals from Sigma (Saint Louis, Mo.) or Tocris (Ballwin, Mo.).

Animals

Male C57BL/6J mice, homozygous mice deficient for PPARα (129S4/SvJae-PPARα a^{tm/Gonz}) and wild-type mice (129S1/SvlmJ) were purchased from the Jackson Laboratory. Male Zucker rats (7 weeks of age) were obtained from Charles River. Male Wistar rats (325±30 g) were from Charles River. Animals were maintained on a 12-h light/dark cycle (light off at 5:30 PM) with water and chow pellets (RMH 2500, Prolab) available ad libitum.

Transactivation Assays

Transactivator plasmids pFA-PPARα, pFA-PPARδ, pFA-PPARγ and pFA-RXR, which encoded for the DNA-binding domain (DBD) of hPPARα<(499-1404), hPPARδ (412-1320), hPPARγ (610-1434) and hRXR (402-1389) fused to the DNA-binding domain (residues 1-147) of yeast GAL4 under control of the human cytomegalovirus (CMV) promoter were generated. The plasmids contained a neomycin-resistance gene to provide stable selection with G418 (200 μg-ml⁻¹; Calbiochem). The HeLa cells were cultured in Dulbecco's-modified Eagles's medium (DMEM) supplemented with fetal bovine serum (10%). The cells were transfected with Fugene 6 (3 μl, Roche) containing the pFR-luc plasmid (1 μg, Stratagene). Eighteen hours following transfection, the culture media was replaced with supplemented DMEM containing hygromycin (100 μg-ml⁻¹, Calbiochem). After 4 weeks in culture, the surviving clones were isolated and analyzed by luciferase assay. The clonal cell line HLR was selected because it demonstrated the highest levels of luciferase activity and transfected it with transactivator plasmids to generate cell lines that also expressed the DNA-binding domain of PPARα (HLR-α), PPARδ (HLR-δ), PPARγ (HLR-γ), and RXR (HLR-rxr). The cells were cultured in supplemented DMEM containing hygromycin and G418. For transactivation assays, cells were seeded in 6-well plates (50,000 cells per well) and incubated for 7 hours in supplemented DMEM containing hygromycin and G418, plus appropriate concentrations of test compounds. Dual-luciferase reporter assay system (Promega) and an MLX Microtiter® plate luminometer (Dynex) were used to determine luciferase activity in cell lysates.

RNA Isolation and cDNA Synthesis

Tissues were stored in RnaLater™ (Ambion), extracted total RNA with TRIzol™ (Invitrogen) and quantified it with Ribogreen™ (Molecular Probes). cDNA was synthesized by using SuperscriptII RNase H-reverse transcriptase (Invitrogen).

Polymerase Chain Reaction (PCR)

Reverse transcription of total RNA (2 μg) was performed using Oligo(dT)₁₂₋₁₈ primer (0.2 μg) for 50 min at 42° C. Real Time Quantitative (RTQ) PCR was conducted using an ABI PRISM 7700 sequence detection system (Applied Biosystems). Primer/probe sets were designed using the Primer Express™ software and gene sequences available from the Genebank™ database. Primers and fluorogenic probes were synthesized by TIB (Adelphia). The primer/probe sequences for the mouse genes were:

| | |
|---|---|
| PPARα, | |
| F: | CTTCCCAAAGCTCCTTCAAAAA, |
| R: | CTGCGCATGCTCCGTG, |
| P: | TGGTGGACCTTCGGCAGCTGG; |
| PPARδ, | |
| F: | GATGACAGTGACCTGGCGCT, |
| R: | AGGCCTGGCCGGTCTC, |
| P: | TTCATCGCGGCCATCATTCTGTGT; |
| PPARγ, | |
| F: | AGTGGAGACCGCCCAGG, |
| R: | GCAGCAGGTTGTCTTGGATGT, |
| P: | TTGCTGAACGTGAAGCCCATCGA; |
| CD36, | |
| F: | CGGCGATGAGAAAGCAGAA, |
| R: | CAACCAGGCCCAGGAGC, |
| P: | TGTTCAGAAACCAAGTGACCGGGAAAATAA; |
| FATP, | |
| F: | GCACAGCAGGTACTACCGCA, |
| R: | GGCGGCACGCATGC, |
| P: | TGCTGCCTTTGGCCACCATTCCTA; |
| I-FABP, | |
| F: | TCACCATCACCTATGGACCCA; |
| R, | TCCAGTTCGCACTCCTCCC; |
| P: | AGTGGTCCGCAATGAGTTCACCCTG; |
| GAPDH, | |
| F: | TCACTGGCATGGCCTTCC, |
| R: | GGCGGCACGTCAGATCC, |
| P: | TTCCTACCCCCAATGTGTCCGTCG. |

RNA levels were normalized by using glyceraldehyde 3-phosphate dehydrogenase (GAPDH) as an internal standard. mRNA levels were measured by generating six-point serial standard curves using mouse total RNA. Estimates of relative mRNA abundance (in arbitrary units) were made by using the C_{T} value (Schmittgen et al., 2000). Relative quantifications of RNAs of interest were made by using the 2^{ΔCT} formula, in which ΔC_{T} was calculated by subtracting the C_{T} value for GAPDH from the C_{T} value for the gene of interest. This formula was validated for each primer/probe set by using six-point serial standard curves.

Feeding Experiments

Acute experiments. Drugs or appropriate vehicles (saline, for CCK-8 and d-fenfluramine; dimethylsulfoxide/saline, 70/30, for all other agents; 4 ml-kg⁻¹; i.p.) were administered at 5:00-5:30 PM to free-feeding mice, which were habituated to the experimental setting. Vehicles exerted no significant effect on feeding. Food intake and feeding microstructure was continuously monitored for 12 h using an automated system (ScriPro Inc, NY) (Gaetani et al., 2003).

Subchronic experiments. Male wild-type and PPARα null mice were fed with a very high-fat diet (60kcal % fat; D12492; Research Diets, NJ). After 7 weeks, body mass indices were 0.355±0.01 g-cm² for wild-type mice (n=13) and 0.408±0.01 g-cm² for PPARA null mice (n=15), indicating that the mice had become obese (Gregoire et al., 2002). The mice were divided into 4 groups (n=7-8 each), and treated them for 4 additional weeks with vehicle (saline/polyethylene glycol/Tween 80, 90/5/5; 1 ml-kg⁻¹) or OEA (5 mg-kg⁻¹, once daily, i.p.). In a separate experiment, obese Zucker rats were treated for 2 weeks with vehicle or OEA (5 mg-kg⁻¹, once daily, i.p.), while maintaining them on a regular rodent chow (RMH 2500, Prolab). Food intake and body weight were measured daily. At the end of the experiments, the animals were fasted overnight, and tissues and blood samples collected for biochemical analyses.

Chronic experiment: In a separate experiment, we treated obese Zucker rats for 2 weeks with vehicle (saline/polyethylene glycol/Tween-80, 90/5/5; 1 ml kg-1, once daily, i.p.) or OEA (5 mg kg-1, once daily, i.p.), while maintaining them on a regular rodent chow (RMH 2500, Prolab). We measured food intake and body weight daily. At the end of the experiments, the animals were fasted overnight, and tissues and blood samples collected for biochemical analyses.

Biochemical Analyses

Lipids were extracted from mouse liver and epidydimal adipose tissue (Bligh and Dyer, 1959) and measured triglycerides with a commercial kit (Sigma). Serum lipids and glucose were measured with an automated Synchron LX® system (Beckman-Coulter).

A. PPAR Modulatory Activity of OEA

The following example exemplifies, using OEA as a model compound, how PPAR binding of OEA-like compounds and OEA modulators can be determined and demonstrates the use of an OEA-like compound or OEA-like modulator as a selective high potency binding agonist of PPARa

To test the possibility that OEA may interact with one or more members of this family of ligand-operated transcription factors (Desvergne and Wahli, 1999; Chawla et al., 2001; Berger and Moller, 2002), modified HeLa cells, which cannot metabolize OEA and other fatty acid ethanolamides (FAE) (Day et al., 2001), were genetically modified to stably express a luciferase reporter gene along with the ligand-binding domain of human PPARα; PPARδ, PPARγ, or retinoid X receptor (RXR) fused to the yeast GAL4 DNA-binding domain (Lazennec et al., 2000). In standard transactivation assays, each of these cell lines responded to appropriate synthetic PPARα agonists (data not shown).

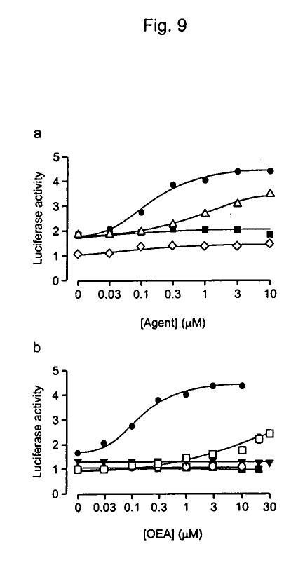

OEA caused a potent activation of PPARα, which was half-maximal at a concentration (EC₅₀) of 120±1 nM (mean±s.e.m., n=16)(FIG. 9A). The compound also activated PPARβ, but less potently than it did PPARα (EC₅₀=1.1±0.1 μM) and had no effect on PPARγ or RXR (FIG. 9A). To explore the structural selectivity of this response, several analogs of OEA were tested for the ability to interact with PPARα. As previously reported (Göttlicher et al., 1992; Kliewer et., 1997; Forman et al., 1997), the parent fatty acid, oleic acid, activated PPARc with micromolar potency (EC₅₀=10.3±0.21 μM; n=16) (FIG. 9B). Conversely, stearylethanolamide, an FAE that contains the same number of carbon atoms as OEA but no double bonds, did not elicit a response (FIG. 9B). Equally ineffective were myristylethanolamide and the endogenous cannabinoid anandamide (arachidonylethanolamide) (Devane et al., 1992) (FIG. 9B). Under the same conditions, the synthetic agonists Wy-14643 (Willson et al., 2000) and GW7647 (Brown et al., 2001) activated PPARα with EC₅₀ values of 1.4±0.1 μM and 150±20 nM, respectively (mean±s.e.m., n=5). The results suggest that OEA activates PPARα in vitro with high potency and selectivity.

B. PPARα Activation and OEA Anorexia

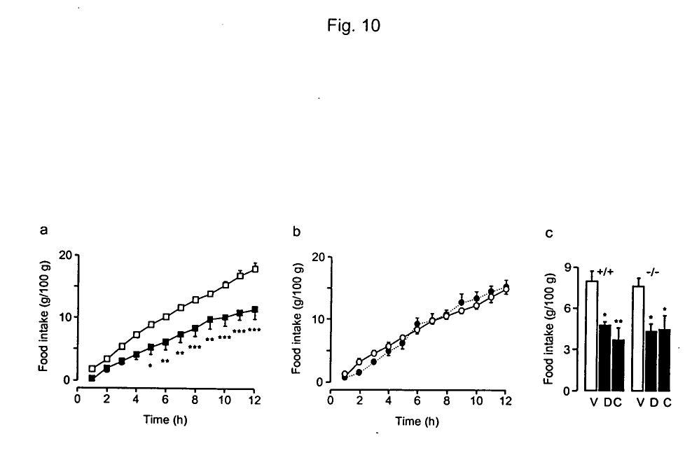

This example illustrates the use of PPARα-null mice to study whether an effect of an OEA-like compound is mediated by the PPARα receptor. To test whether PPARα activation contributes to the anorexiant properties of OEA, mutant mice were used in which the ligand-binding domain of PPARα had been disrupted by homologous recombination (Lee et al., 1995). Homozygous PPARα-null mice are fertile and viable, but do not respond to PPARα agonists and develop late-onset obesity (Lee et al., 1995; Butler and Cone, 2001). Administration of OEA (10 mg-kg⁻¹, intraperitoneal, i.p.) reduced feeding in wild-type mice (FIG. 10A). This effect was absent in PPARα-deficient animals (FIG. 10B), which displayed OEA drug levels (Table 5) comparable to those of wild-type controls and responded normally, however, to the serotonergic anorexiant d-fenfluramine and the peptide hormone cholecystokinin-octapeptide (CCK-8)(FIG. 10C). The effect of OEA was absent in PPAR-α-deficient animals (FIG. 2b), which displayed OEA drug levels comparable to those of wild-type controls (Supplementary Table 1) and responded normally to the serotonergic anorexiant d-fenfluramine and the peptide hormone cholecystokinin-octapeptide (CCK-8) (FIG. 2c).

TABLE 5
OEA levels in the liver of wild-type and PPAR-α^{-/-} mice.

| | Vehicle | OEA |
|---|---|---|
| Wild-type | 72.8 ± 12.1 | 150.5 ± 19.7 |
| PPAR-a-null | 73.5 ± 1.6 | 251.2 ± 28.2 |

OEA (5 mg kg⁻¹, i.p.) or vehicle was administered by i.p. injection. Liver OEA content was measured 1 h after administration by HPLC/MS. Results are expressed in pmol-g⁻¹ and are the mean ± sem of n = 3-4.

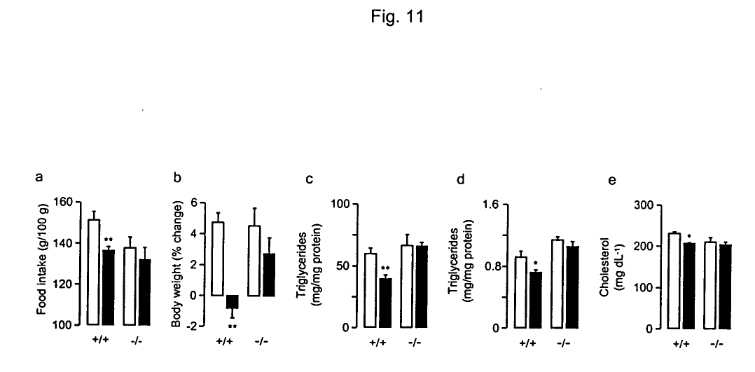

To determine the role of PPARt on the effect of subchronically administered OEA in rats in producing a sustained inhibition of food intake and inhibition of body-weight gain, wild-type and PPARα deficient mice were fed with a high-fat chow for 7 weeks to induce obesity, and treated them for 4 subsequent weeks with daily injections of vehicle or OEA (5 mg-kg⁻¹, i.p.). In obese wild-type mice, OEA significantly reduced cumulative food intake (normalized for body mass) (FIG. 11A) and suppressed body-weight gain (FIG. 11B). By contrast, no such effect was observed in obese PPARα deficient animals (FIG. 11A-B). These results suggest that expression of a functional PPARα is necessary for the satiety-inducing and weight-reducing actions of OEA. They also illustrate the use of PPAR-α null mammals to determine the receptor mechanism of an OEA-like compound.

C. High Potency Selective PPARα Agonist Compounds are Required to Affect Appetite and Body Weight Gain.

This example illustrates the screening of and use of compounds which are high affinity agonists for use in treating anorexia and to reduce body weight or body fat. The possibility that OEA modulates feeding through direct activation of PPARα was further investigated despite the fact that this possibility seemed negated by the fact that fibric acids, a class of PPARα agonists that is widely used in the therapy of hyperlipidemias, do not notably affect food intake (Best and Jenkins, 2001). Fibric acids are, however, 200 to 900 times less potent than OEA at activating PPARα (Willson et al., 2000).

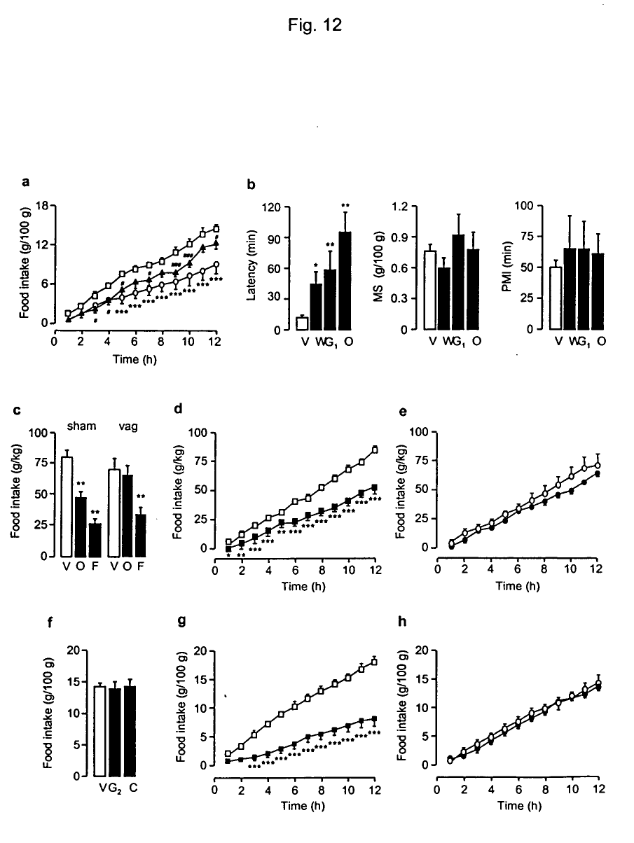

Therefore, to assess the contribution of PPAR-α to feeding regulation, compounds with potencies comparable to that of OEA were used: Wy-14643 (see, Willson, T. M., Brown, P. J., Sternbach, D. D. & Henke, B. R. J Med Chem 43, 527-50. (2000)) and GW7647 (see Brown, P. J. et al. in PCT Int. Appl. 32 (2000)). Both drugs inhibited food intake in C57BL/6J mice (FIG. 12a), whereas the fibric acid derivative clofibrate did not (25-100 mg kg⁻¹; data not shown). Meal pattern analyses revealed that the anorexiant effects of Wy-14643 and GW7647 were due to a selective prolongation of eating latency rather than to changes in meal size or post-meal interval (FIG. 12b). This response is essentially identical to that elicited by OEA (10 mg kg⁻¹, i.p.) (FIG. 12b) and is suggestive of a satiety-inducing action.

OEA is thought to produce satiety by activating visceral sensory fibres (see, Rodriguez de Fonseca, F. et al., Nature 414, 209-12. (2001). Accordingly, in rats in which these fibres had been removed either by severing the vagus nerve below the diaphragm or by capsaicin treatment, OEA (10 mg kg-1, i.p.) had no effect on food intake (FIG. 12c). These procedures also prevented the hypophagic effects of Wy-14643 (40 mg kg-1, i.p.) (FIG. 12d-e and Table 6), but not those of the centrally acting anorexiant d-fenfluramine (FIG. 12c)

TABLE 6
Effects of sensory deafferentation on the anorexiant
responses to Wy-14643.

| | | | |
|---|---|---|---|
| 30 min | 60 min | 120 min | 240 min |

Control rats

| | | | | |
|---|---|---|---|---|
| Vehicle | 5.6 ± 0.8 | 6.5 ± 0.9 | 7.6 ± 0.8 | 10.2 ± 0.9 |
| Wy-14643 | 2.3 ± 1.3* | 3.6 ± 1.2* | 5.8 ± 1.3* | 6.4 ± 1.8* |

Capsaicin-treated rats

| Vehicle | 2.9 ± 0.9 | 4.5 ± 0.9 | 6.7 ± 0.8 | 8.8 ± 0.8 |
|---|---|---|---|---|
| Wy-14643 | 2.2 ± 1.2 | 3.7 ± 1.6 | 4.9 ± 1.5 | 7.8 ± 1.9 |
| Wy-14643 (40 mg kg⁻¹, i.p.) or vehicle was administered to 24-h food-deprived Wistar rats (325 ± 30 g) and food intake was measured manually. Results are the mean ± sem of n = 6. Asterisk, P < 0.05 vs vehicle. Capsaicin deafferentation. Male Wistar rats were treated with capsaicin or vehicle, as described¹. The animals were habituated to handling, food-deprived for 24 h and given Wy-14643 or vehicle (DMSO/saline, 70/30). Food | | | | |
| # pellets and spillage were measured manually 30-240 min after drug injection. | | | | |

The close correspondence between the effects of OEA and those of synthetic PPAR-α agonists suggests that OEA modulates feeding through activation of PPAR-α. This conclusion is reinforced by two findings. First, potent agonists at PPAR-P/6 (GW501516; 1-10 mg kg⁻¹, i.p.) (see, Oliver, W. R., Jr. et al., Proc Natl Acad Sci USA 98, 5306-11. (2001). and PPAR-γ (ciglitazone; 15 mg kg⁻¹, i.p.) (see, Chang, A. Y., Wyse, B. M., Gilchrist, B. J., Peterson, T. & Diani, A. R., Diabetes 32, 830-8. (1983)) did not affect feeding in C57BL/6J mice (FIG. 3f); and, second, mice deficient in PPAR-α did not respond to Wy-14643 (40 mg kg⁻¹, i.p.) (FIG. 3g-h). OEA has slight PPARβ activity. As the PPAR-β/δ agonist GW501516 does not affect food intake, and OEA does not induce satiety or weight reduction in PPAR-α null mice, the data indicate that the any role of PPAR-β/δ in OEA signalling is, if any, distinct from that of PPAR-α.

D. OEA Initiation of PPARα Gene Expression.

The above result was unexpected, because the actions of PPARα were thought to be mediated through transcriptional regulation of gene expression (Desvergne and Wahli, 1999; Chawla et al., 2001; Berger and Moller, 2002), which was considered too slow to account for the rapid satiety-inducing effects of OEA.

Therefore, to further test the hypothesis that OEA activates PPARα, the ability of the compound to initiate expression of PPARα-regulated genes was investigated first, on the small intestine, which is one of the most likely sites of action of OEA (see, Rodriguez de Fonseca, F. et al., Nature 414, 209-12. (2001)) and contains high levels of PPARα (see, Escher, P. et al., Endocrinology 142, 4195-202. (2001).

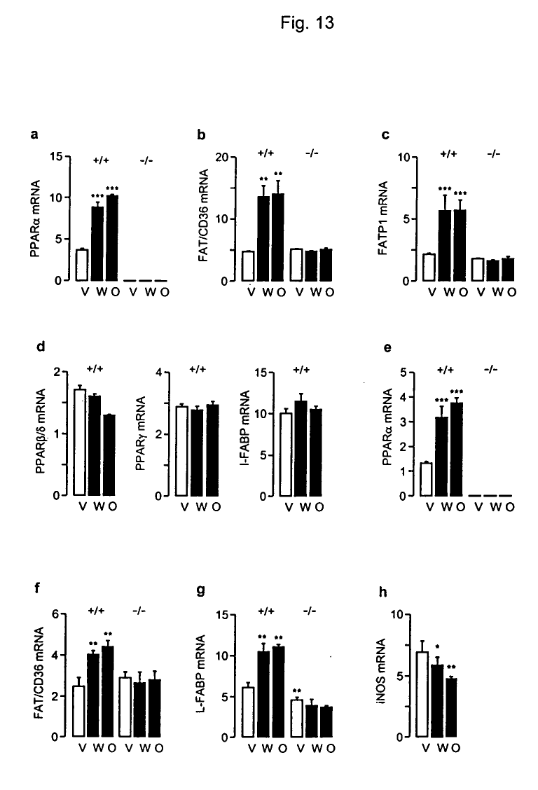

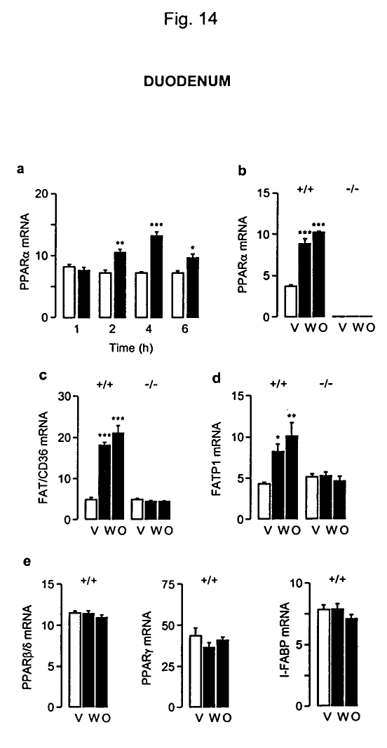

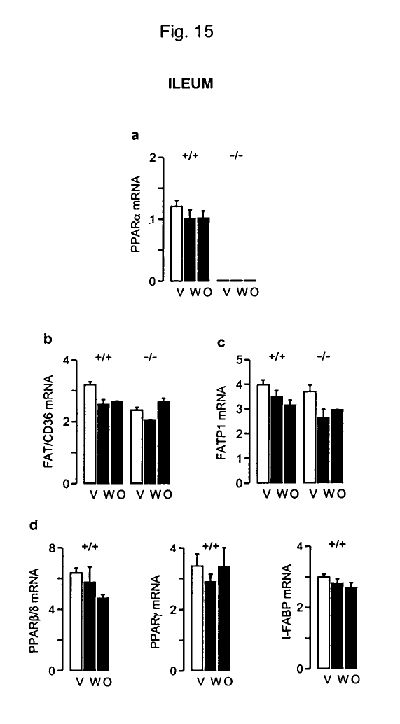

In the jejunum of wild-type mice, OEA (10 mg kg⁻¹, i.p.), but not oleic acid (10 mg kg⁻¹, i.p.; data not shown), increased the expression of three PPAR-α-regulated genes: PPAR-α itself (FIG. 13a), fatty acid translocase (FAT/CD36) (FIG. 13b) and fatty acid transport protein 1 (FATP1) (FIG. 13c) (see, Martin, G., Schoonjans, K., Lefebvre, A. M., Staels, B. & Auwerx, J., J Biol Chem 272, 28210-7. (1997) and Motojima, K., Passilly, P., Peters, J. M., Gonzalez, F. J. & Latruffe, N., J Biol Chem 273, 16710-4. (1998)). Interestingly, a similar stimulatory effect was observed in the duodenum (FIG. 14) which, like the jejunum, plays a key role in fatty acid absorption, but not in the ileum (FIG. 15), which is primarily involved in the absorption of cholesterol and bile salts. By contrast, the expression of three related genes, which are not under the control of PPAR-α (intestinal fatty acid-binding protein, I-FABP, PPAR-β/δ and PPAR-γ) was not affected by OEA either in wild-type (FIG. 13d) or PPAR-α-null mice (data not shown). Underscoring the role of PPAR-α in these responses, it was found that (i) the PPAR-α agonist Wy-14643 (30 mg kg⁻¹, i.p.) mimicked the effects of OEA (FIG. 13a-d), and (ii) OEA and Wy-14643 did not stimulate gene expression in mice deficient in PPAR-α (FIG. 13a-c). The ability of OEA to activate PPAR-α-mediated gene expression was not restricted to the intestine, as the compound also initiated transcription of PPAR-α-regulated genes in the liver of wild-type, but not PPAR-α-null mice (FIG. 13e-g).

In addition to stimulating transcription, PPAR-A activation also is known to induce the transrepression of various genes, such as inducible nitric-oxide synthase (iNOS) (see Colville-Nash, P. R., Qureshi, S. S. & Willoughby, D.; J Immunology 161, 978-984 (1998)). Accordingly, in the jejunum of C57BL/6J mice, administration of OEA (10 mg kg⁻¹, i.p.) or Wy-14643 (30 mg kg⁻¹, i.p.) significantly decreased iNOS expression (FIG. 13h), whereas oleic acid (10 mg kg⁻¹, i.p.) was ineffective (data not shown). These results indicate that OEA closely mimics the genomic actions of PPAR-A agonists in a PPAR-α-dependent manner.

E. Effect of OEA on Serum Lipids.

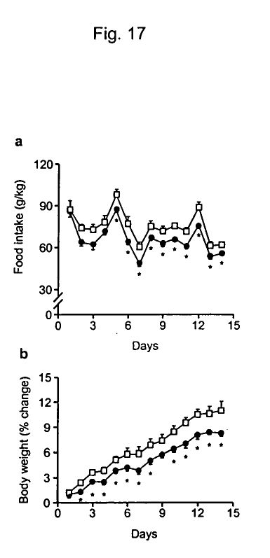

This example illustrates the use of an OEA-like compound to reduce serum lipids. If OEA enhances expression of PPARα-regulated genes, it also should reproduce the metabolic consequences of long-term treatment with PPARα agonists, a prominent example of which is the reduction of genetic or diet-induced hyperlipidemia (see, Best, J. D. & Jenkins, A. J., Expert Opin Investig Drugs 10, 1901-11. (2001)). Consistent with this prediction, OEA treatment (5 mg-kg⁻¹, once daily for 2 weeks, i.p.) reduced fasting serum cholesterol and triglyceride levels in genetically obese Zucker (fal/fa) rats (Table 7). These effects were accompanied by a significant inhibition of food intake and body-weight gain (FIG. 17) and were qualitatively similar to those previously reported for the PPARα agonists clofibrate and fenofibrate (see, Cleary, et al. Atherosclerosis 66, 107-12. (1987) and Chaput, E., et al. Biochem Biophys Res Commun 271, 445-50. (2000)). Furthermore, high fat-fed wild-type and PPARα-null mice develop hypercholesterolemia, but maintain normal serum triglyceride levels (in mg-dl⁻¹; wild-type, cholesterol: 253±7; triglycerides: 72±3; PPARα-null, cholesterol: 216±11; triglycerides: 82±9 mg-dl⁻¹; n=8-9). A 4-week OEA regimen (5 mg-kg⁻¹, once daily, i.p.) partially corrected this alteration in wild-type mice, but was ineffective in PPARα-null animals (FIG. 11C). These findings indicate that long-term administration of OEA induces metabolic changes, which are reminiscent of those elicited by PPARα agonists and are abrogated by deletion of PPARα.

TABLE 7
Effects of OEA on serum lipids and glucose in obese Zucker
rats. OEA (5 mg kg−1, i.p.) or vehicle was administered once a
day for 2 weeks. Serum cholesterol, triglycerides and glucose
were measured and are expressed in mg dl−1. Results are the
mean ± sem of n = 7-8. Asterisk, P < 0.05 vs vehicle.

| | Vehicle | OEA |
|---|---|---|
| Cholesterol | 99.88 ± 8.41 | 66.14 ± 7.06* |
| Triglycerides | 565.29 ± 55.50 | 394.17 ± 49.40* |
| Glucose | 229.29 ± 27.90 | 221.25 ± 23.80 |

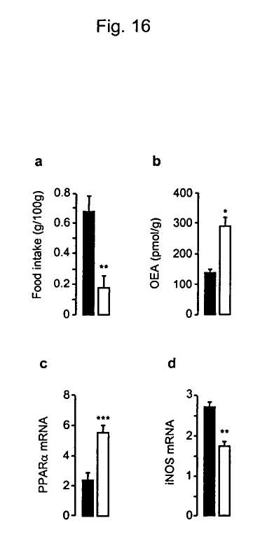

The ability of OEA to activate PPARα in vitro, the close similarity between its pharmacological properties and those of PPARα agonists, and the lack of such effects in PPARα null mice, indicate that OEA is a natural ligand for PPARα. The concerted regulation of OEA synthesis and PPAR-α/iNOS expression further supports this possibility. In the small intestine of C57BL/6J mice, OEA levels were significantly lower at night (1:30 AM), when the animals are actively engaged in feeding, than during the day (4:30 PM), when they are satiated and resting (FIG. 16a-b). Intestinal PPAR-α expression paralleled OEA levels (FIG. 16c), whereas expression of the PPAR-A transrepression target, iNOS, displayed an opposite pattern (FIG. 16d). Importantly, the diurnal concentrations of OEA in intestinal tissue (=300 nM) were in the range needed to fully activate PPAR-α in vitro (EC₅₀=120 nM), suggesting that they may be adequate to engage this receptor and regulate transcription of its target genes in vivo.

In conclusion, these results indicate that OEA is the first natural compound that meets all key criteria for it to be considered an endogenous PPAR-α ligand: (i) it binds with nanomolar affinity to mouse and human PPAR-α; (ii) it mimics the actions of synthetic PPAR-α agonists in a PPAR-α-dependent manner; and (iii) it reaches, under appropriate physiological conditions, tissue levels that are sufficiently high to activate PPAR-α. Furthermore, the findings suggest that PPAR-α activation does not only mediate OEA-induced weight stabilisation, which is expected from the metabolic roles of this receptor (see, Desvergne, B. & Wahli, W., Endocr Rev 20, 649-88. (1999), Chawla, A., et al., Science 294, 1866-70. (2001), and Berger, J. & Moller, D. E., Annu Rev Med 53, 409-35. (2002)), but also is responsible for OEA-induced satiety, a behavioural role that was not previously attributed to PPAR-α. The molecular mechanism underlying this response is still undefined, but one possibility is that it may involve the regulation of intestinal NO production. Intestinal epithelial cells express the NO-synthesizng enzyme, iNOS, and generate significant amounts of this gaseous messager, which is thought to act as a peripheral orexigenic signal (see, Colville-Nash, P. R., et al., J Immunology 161, 978-984 (1998), Sticker-krongrad, A., et al., Life Sci 58, PL9-15 (1996), and Janero, D. R., Nutrition 17, 896-903 (2001). The ability of OEA to transrepress iNOS via PPAR-α suggests that iNOS down-regulation may contribute to the persistent anorexiant actions of OEA. Irrespective of these speculations, our study identifies OEA as a primary endogenous agonist for PPAR-α and opens new perspectives for the treatment of eating disorders.

### Example 4

### Methods for Identifying an OEA-Like Compound or an OEA-Like Modulator for Use in Modulating Appetite, Reducing Body Fat, or Regulating Fat Metabolism

An OEA-like compound or modulator for reducing body fat in a mammal can be identified by screening one or more OEA-like compounds or candidate OEA-like modulators in a binding or activation assay for each of PPARα, PPARβ and PPARγ and selecting the compound for further testing if it is a specific agonist of peroxisome proliferator activated receptor type a (PPARα) having at least a 5 fold specificity for PPARα over both PPARγ and PPARβ and produces a half-maximal effect on PPARα at a concentration of less than 1 micromolar; and then testing the compound selected in step (i) by administering the compounds to the mammal and determining, as compared to an appropriate vehicle control, the amount of body fat reduction, appetite suppression, or fat metabolism alteration.

### Example 5

### Exemplary FAAH Inhibitors for Use in Treating a Disease or Condition Mediated by PPARα or Responsive to Therapy by a PPARα Agonist

Trifluoroketone inhibitors such as the compound of Formula VII are also contemplated for use in inhibiting FAAH to raise endogenous levels of OEAor treat the subject conditions and disorders.

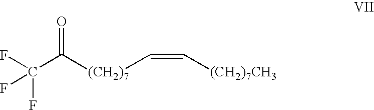

Such compounds are taught in U.S. Pat. No. 6,096,784 herein incorporated by reference.

Other compounds for use according to the invention include octylsulfonyl and octylphosphonyl compounds. See Quistand et al. in Toxicology and Applied Pharmacology 179: 57-63 (2002). See also Quistand et al. in Toxicology and Applied Pharmacology 173: 48-55 (2001).

Other compounds for use according to the invention include the alpha-keto-oxazolpyridines which are reversible and extremely potent inhibitiors of FAAH. See Boger et al., PNAS USA 97:5044-49 (2000). Exemplary compounds include compounds of the Formula: 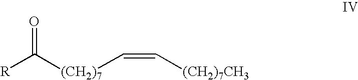
wherein R is an alpha-keto oxazolopyridinyl moiety such as

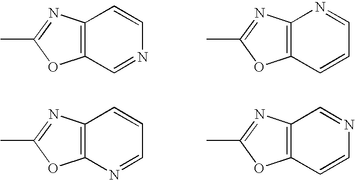

Boger et al. teach other exemplary compounds of the invention including substituted alpha-keto-heterocycle analogs of fatty acid amides. In particular, wherein R is an alpha-keto oxazolopyridinyl moiety and the fatty acid moiety is a homolog of oleic acid or arachidonic acid.

Other FAAH inhibitors for use according to the invention include fatty acid sulfonyl fluorides such as compound AM374 which irreversibly binds FAAH. See Deutsch et al., Biochem. Biophys Res Commun. 231:217-221 (1997).

Other preferred FAAH inhibitors include, but are not limited to, the carbamate FAAH inhibitors disclosed in Kathuria et al., Nat Med January; 9(1):76-81(2003) incorporated herein by reference for the FAAH inhibitor compounds it discloses. Particularly preferred are selective FAAH inhibitors such as URB532 and URB597 disclosed therein.

### Example 6

### Methods of Screening Compounds for FAAH Inhibitory Activity

Methods for screening compounds for FAAH inhibitory activity in vitro are well known to one of ordinary skill in the art. Such methods are taught in Quistand et al. in Toxicology and Applied Pharmacology 179: 57-63 (2002); Quistand et al. in Toxicology and Applied Pharmacology 173: 48-55 (2001); Boger et al., PNAS USA 97:5044-49 (2000).

Methods for screening compounds for FAAH inhibitory activity in vivo and increased endogenous cannabinoid levels or activity are known to one of ordinary skill in the art. Such methods include measurement of fatty acid ethanolamides in tissue and are taught in Quistand et al. in Toxicology and Applied Pharmacology 179: 57-63 (2002); Quistand et al. in Toxicology and Applied Pharmacology 173: 48-55 (2001); Boger et al., PNAS USA 97:5044-49 (2000). See U.S. Pat. No. 6,096,784. See also PCT Publication WO 98/24396. See Cravatt et al. PNAS 98:9371-9376 (2001).

### Example 7

### Exemplary OEA-Like Compounds and/or OEA-Like Modulators

In some embodiments, specific PPARα agonists are used to modulate appetite or reduce body fat or to alter fat metabolism. Selective high affinity PPARα agonists are well known in the art. Exemplary OEA-like modulators include GW 7647 and GW501516. PPARα modulators are taught in U.S. Pat. No. 6,468,996; U.S. Pat. No. 6,465,497; U.S. Pat. No. 6,534,517; U.S. Pat. No. 6,506,781; U.S. Pat. No. 6,407,127; and U.S. Pat. No. 6,200,998. The disclosures of each of which are herein incorporated by reference with particular respect to the subject matter of the PPAR modulatory compounds they disclose and only to the extent not inconsistent with the present specification. Specific PPAR agonists can be ascertained by use of a PPAR activation assay panel of PPARα, PPARγ, and PPARβ.

### REFERENCES

1. Rodriguez de Fonseca, F. et al. An anorexic lipid mediator regulated by feeding. Nature 414, 209-12. (2001).
2. Gaetani, S., Oveisi, F. & Piomelli, D. Modulation of meal pattern in the rat by the anorexic lipid mediator oleoylethanolamide. Neuropsycopharmacology, (in press) (2003).
3. Göttlicher, M., Widmark, E., Li, Q. & Gustafsson, J. A. Fatty acids activate a chimera of the clofibric acid-activated receptor and the glucocorticoid receptor. Proc Natl Acad Sci U S A 89, 4653-7. (1992).
4. Kliewer, S. A. et al. Fatty acids and eicosanoids regulate gene expression through direct interactions with peroxisome proliferator-activated receptors. Proc Natl Acad Sci USA 94, 4318-23. (1997).
5. Forman, B. M., Chen, J. & Evans, R. M. Hypolipidemic drugs, polyunsaturated fatty acids, and eicosanoids are ligands for peroxisome proliferator-activated receptors. Proc Natl Acad Sci USA 94, 4312-7. (1997).
6. Desvergne, B. & Wahli, W. Peroxisome proliferator-activated receptors: nuclear control of metabolism. Endocr Rev 20, 649-88. (1999).
7. Chawla, A., Repa, J. J., Evans, R. M. & Mangelsdorf, D. J. Nuclear receptors and lipid physiology: opening the X-files. Science 294, 1866-70. (2001).
8. Berger, J. & Moller, D. E. The mechanisms of action of PPARs. Annu Rev Med 53, 409-35. (2002).
9. Day, T. A., Rakhshan, F., Deutsch, D. G. & Barker, E. L. Role of fatty acid amide hydrolase in the transport of the endogenous cannabinoid anandamide. Mol Pharmacol 59, 1369-75. (2001).
10. Lazennec, G., Canaple, L., Saugy, D. & Wahli, W. Activation of peroxisome proliferator-activated receptors (PPARs) by their ligands and protein kinase A activators. Mol Endocrinol 14, 1962-75. (2000).
11. Devane, W. A. et al. Isolation and structure of a brain constituent that binds to the cannabinoid receptor. Science 258, 1946-9 (1992).
12. Willson, T. M., Brown, P. J., Sternbach, D. D. & Henke, B. R. The PPARs: from orphan receptors to drug discovery. J Med Chem 43, 527-50. (2000).
13. Brown, P. J. et al. in PCT Int. Appl. 32 (2000).
14. Lee, S. S. et al., Mol Cell Biol 15, 3012-22. (1995).
15. Butler, A. A. & Cone, R. D. Knockout models resulting in the development of obesity. Trends Genet 17, S50-4. (2001).
16. Oliver, W. R., Jr. et al., Proc Natl Acad Sci USA 98, 5306-11. (2001).
17. Chang, A. Y., Wyse, B. M., Gilchrist, B. J., Peterson, T. & Diani, A. R. Ciglitazone, a new hypoglycemic agent. I. Studies in ob/ob and db/db mice, diabetic Chinese hamsters, and normal and streptozotocin-diabetic rats. Diabetes 32, 830-8. (1983).
18. Escher, P. et al. Rat PPARs: quantitative analysis in adult rat tissues and regulation in fasting and refeeding. Endocrinology 142, 4195-202. (2001).
19. Martin, G., Schoonjans, K., Lefebvre, A. M., Staels, B. & Auwerx, J. Coordinate regulation of the expression of the fatty acid transport protein and acyl-CoA synthetase genes . . . J Biol Chem 272, 28210-7. (1997).
20. Motojima, K., Passilly, P., Peters, J. M., González, F. J. & Latruffe, N., J Biol Chem 273, 16710-4. (1998).
21. Colville-Nash, P. R., Qureshi, S. S. & Willoughby, D. A. Inhibition of inducible nitric oxide synthase by peroxisome proliferatior-activated receptor agonist: correlation of induction of heme ocygenasel. J Immunology 161, 978-984 (1998).
22. Best, J. D. & Jenkins, A. J. Novel agents for managing dyslipidaemia. Expert Opin Investig Drugs 10, 1901-11. (2001).
23. Cleary, M. P., Kasiske, B., O'Donnell, M. P. & Keane, W. F. Effect of long-term clofibric acid treatment on serum and tissue lipid and cholesterol levels in obese Zucker rats. Atherosclerosis 66, 107-12. (1987).
24. Chaput, E., Saladin, R., Silvestre, M. & Edgar, A. D. Fenofibrate and rosiglitazone lower serum triglycerides with opposing effects on body weight. Biochem Biophys Res Commun 271, 445-50. (2000).
25. Akiyama, T. E. et al., J Biol Chem 276, 39088-93. (2001).
26. Sticker-krongrad, A., Beck, B. & Burlet, C. Nitric oxide mediates hyperphagia of obese zucker rats: relation to specific changes in the microstructure of feeding behavior. Life Sci 58, PL9-15 (1996).
27. Janero, D. R. Nutitional aspects of nitric oxide: human health implication and therapeutic opportunities. Nutrition 17, 896-903 (2001).
28. Chao, E. Y.-H. et al. in PCT Int. Appl. 83 (2001).
29. Giuffrida, A., Rodriguez de Fonseca, F. & Piomelli, D. Quantification of bioactive acylethanolamides in rat plasma by electrospray mass spectrometry. Anal Biochem 280, 87-93 (2000).
30. Schmittgen, T. D. et al. Quantitative reverse transcription-polymerase chain reaction to study mRNA decay: comparison of endpoint and real-time methods. Anal Biochem 285, 194-204. (2000).
31. Gregoire, F. M. et al. Diet-induced obesity and hepatic gene expression alterations in C57BL/6J and ICAM-1-deficient mice. Am J Physiol Endocrinol Metab 282, E703-13. (2002).

All publications and patent applications cited in this specification are herein incorporated by reference to the extent not inconsistent with the present disclosure as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for modulating the PPARt receptor in a subject, said method comprising administering an OEA-like compound.

2. The method of claim 1, wherein the compound satisfies the formula: 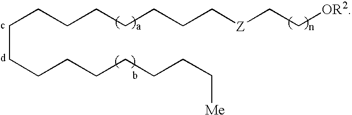
wherein n is from 0 to 5, the sum of a and b can be from 0 to 4; Z is a member selected from the group consisting of —C(O)N(R^{o})—; —(R^{o})NC(O)—; —OC(O)—; —(O)CO—; O;
NR^{o}; and S; and wherein R^{o} and R² are members independently selected from the group consisting of unsubstituted or unsubstituted alkyl, hydrogen, C₁-C₆ alkyl, and lower (C₁-C₆) acyl, and wherein up to eight hydrogen atoms are optionally substituted by methyl or a double bond, and the bond between carbons c and d may be unsaturated or saturated,
or a pharmaceutically acceptable salt thereof.

3. The method of claim 2, wherein the compound satisfies the formula: 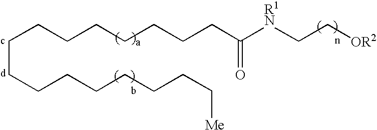
wherein n is from 0 to 4, the sum of a and b is from 1 to 3, and R¹ and R² are members independently selected from the group comprising hydrogen, C₁-C₆ alkyl, and lower (C₁-C₆) acyl, and wherein up to eight hydrogen atoms are optionally substituted by methyl or a double bond, and the bond between carbons c and d may be unsaturated or saturated;
or a pharmaceutically acceptable salt thereof.

4. The method of claim 2, wherein a=1 and b=1.

5. The method of claim 2, wherein n=1.

6. The method of claim 2, wherein R¹ and R² are each H.

7. The method of claim 2, wherein the bond between carbon c and carbon d is a double bond.

8. The method of claim 1, wherein the compound is [2-Methyl-4-[4-methyl-2-(4-trifluoromethyl-phenyl)-thiazol-5-ylmethylsulfanyl]-phenoxy]-acetic acid or a pharmaceutically acceptable salt thereof.

9. The method of claim 1, wherein the compound is 2-(4-{2-[3-Cyclohexyl-1-(4-cyclohexyl-butyl)-ureido]-ethyl}-phenylsulfanyl)-2-methyl-propionic acid or a pharmaceutically acceptable salt thereof.

10. The method of claim 1, wherein the administering is parenteral, oral, intravenous, topical, local, transdermal, rectal, or intranasal.

11. The method of claim 1, wherein the subject is human.

12. A method for treating an immune disorder in a subject, said method comprising administering to the subject an OEA-like compound.

13. The method of claim 12, wherein the compound satisfies formula: 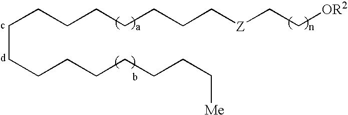
wherein n is from 0 to 5, the sum of a and b can be from 0 to 4; Z is a member selected from the group consisting of —C(O)N(R^{o})—; —(R^{o})NC(O)—; —OC(O)—; —(O)CO—; O;
NR^{o}; and S; and wherein R^{o} and R² are members independently selected from the group consisting of unsubstituted or unsubstituted alkyl, hydrogen, C₁-C₆ alkyl, and lower (C₁-C₆) acyl, and wherein up to eight hydrogen atoms are optionally substituted by methyl or a double bond, and the bond between carbons c and d may be unsaturated or saturated,
or a pharmaceutically acceptable salt thereof.

14. The method of claim 12, wherein the compound satisfies the formula: 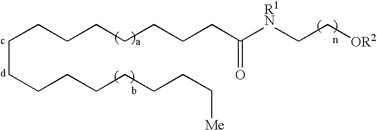
wherein n is from 0 to 4, the sum of a and b is from 1 to 3, and R1 and R2 are members independently selected from the group comprising hydrogen, C₁-C₆ alkyl, and lower (C₁-C₆) acyl, and wherein up to eight hydrogen atoms are optionally substituted by methyl or a double bond, and the bond between carbons c and d may be unsaturated or saturated;
or a pharmaceutically acceptable salt thereof.

15. The method of claim 13, wherein a=1 and b=1.

16. The method of claim 13, wherein n=1.

17. The method of claim 13, wherein R1 and R2 are each H.

18. The method of claim 13, wherein the bond between carbon c and carbon d is a double bond.

19. The method of claim 12, wherein the compound is [2-Methyl-4-[4-methyl-2-(4-trifluoromethyl-phenyl)-thiazol-5-ylmethylsulfanyl]-phenoxy]-acetic acid or a pharmaceutically acceptable salt thereof.

20. The method of claim 12, wherein the compound is 2-(4-{2-[3-Cyclohexyl-1-(4-cyclohexyl-butyl)-ureido]-ethyl}-phenylsulfanyl)-2-methyl-propionic acid or a pharmaceutically acceptable salt thereof.

21. The method of claim 12, wherein the administering is parenteral, oral, transdermal, rectal, or intranasal.

22. The method of claim 12, wherein the subject is human.

23. A method of treating a disease or condition mediated by PPARα, said method comprising administering an OEA-like compound.

24. A method of claim 23, wherein the compound satisfies the formula: 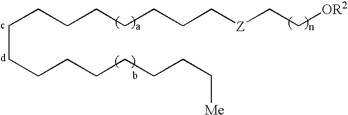
wherein n is from 0 to 5, the sum of a and b can be from 0 to 4; Z is a member selected from the group consisting of —C(O)N(R^{o})—; —(R)NC(O)—; —OC(O)—; —(O)CO—; O;
NR^{o}; and S; and wherein R^{o} and R² are members independently selected from the group consisting of unsubstituted or unsubstituted alkyl, hydrogen, C₁-C₆ alkyl, and lower (C₁-C₆) acyl, and wherein up to eight hydrogen atoms are optionally substituted by methyl or a double bond, and the bond between carbons c and d may be unsaturated or saturated,
or a pharmaceutically acceptable salt thereof.

25. The method of claim 24, wherein the compound satisfies the formula: 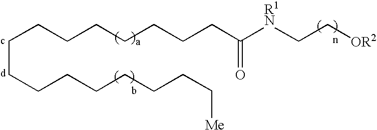
wherein n is from 0 to 4, the sum of a and b is from 1 to 3, and R1 and R2 are members independently selected from the group comprising hydrogen, C1-C6 alkyl, and lower (C1-C6) acyl, and wherein up to eight hydrogen atoms are optionally substituted by methyl or a double bond, and the bond between carbons c and d may be unsaturated or saturated;
or a pharmaceutically acceptable salt thereof.

26. The method of claim 24, wherein a=1 and b=1.

27. The method of claim 24, wherein n=1.

28. The method of claim 24, wherein R1 and R2 are each H.

29. The method of claim 24, wherein the bond between carbon c and carbon d is a double bond.

30. The method of claim 23, wherein the administering is parenteral, oral, intravenous, transdermal, rectal, or intranasal.

31. The method of claim 23, wherein the subject is human.

32. The method of claim 23 wherein the disease or condition is inflammation of a joint or tissue.

33. The method of claim 23, wherein the disease or condition is selected from the group consisting of Alzheimer's disease, Crohn's disease, a vascular inflammation, an inflammatory bowel disorder, artherogenesis, rheumatoid arthritis, asthma, and thrombosis.

34. The method of claim 23, wherein the disease or condition is a metabolic disorder.

35. The method of claim 23, wherein the disease or condition is selected from the group consisting of hyperlipidemia, Type II diabetes, insulin resistance, hypercholesterolemia, and hypertriglyceridemia.

36. The method of claim 23, wherein the modulator is a specific PPARα agonist.

37. A method of screening fatty acid alkanolamides for their ability to modulate appetite, metabolism, blood lipids, or an inflammatory disorder, said method comprising:
contacting the fatty acid alkanolamide in vitro with a PPARα receptor; and
detecting the ability of the fatty acid alkanolamide to bind the receptor.

38. The method of claim 37, wherein the detecting detects a cellular transduction signal of a PPARα agonist.

39. The method of claim 37, wherein the detecting detects binding of the fatty acid alkanolamide to PPARα.

40. A method of treating a condition mediated by a PPARα, said method comprising administering a FAAH inhibitor to a subject having the condition.

41. The method of claim 40, wherein the condition is obesity.

42. The method of claim 40, wherein the condition is inflammation.

43. The method of claim 40, wherein the condition is a hyperlipidemia.

44. The method of claim 40, wherein the condition is diabetes mellitus.

45. The method of claim 40, wherein the condition is selected from the group consisting of Alzheimers disease, Crohn's disease, a vascular inflammation, an inflammatory bowel disorder, artherogenesis, rheumatoid arthritis, asthma, and thrombosis.

46. The method of claim 40, wherein the condition is consisting of hypercholesterolemia or hypertriglyceridemia.

47. A method of treating obesity, said method comprising administering an OEA-like modulator.

48. A method of treating an appetite disorder, said method comprising administering an OEA-like modulator.

49. A method of reducing body fat, said method comprising administering an OEA-like modulator.

50. A method of treating cellulite, said method comprising administering an OEA-like compound.

51. A method of treating cellulite, said method comprising administering an OEA-like modulator.

52. A method of claim 50 or 51, wherein said administering is local.

53. A method of claim 50 or 51, wherein said administering is topical.
